(19) United States
(12) Patent Application Publication
(10) Pub. No.: US 2023/0088070 A1
(43) Pub. Date: Mar. 23, 2023

(54) USE OF IL-1BETA BINDING ANTIBODIES

(71) Applicant: NOVARTIS AG, Basel (CH)

(72) Inventors: Andreas BRUEDERLE, Basel (CH); Anshu MARATHE, East Hanover, NJ (US); Jean REGARD, Cambridge, MA (US); Mikael Lee RINNE, Brookline, MA (US); Haiying SUN, Montville, NJ (US); K. Gary J. VANASSE, Wellesley, MA (US); Connie WONG, Arlington, MA (US)

(21) Appl. No.: 17/728,265

(22) Filed: Apr. 25, 2022

Related U.S. Application Data
(63) Continuation of application No. 16/719,154, filed on Dec. 18, 2019, now abandoned.
(60) Provisional application No. 62/783,568, filed on Dec. 21, 2018.

(51) Int. Cl. C07K 16/24; A61P 35/04; C07K 16/28

(52) U.S. Cl. CPC C07K 16/245; A61P 35/04; C07K 16/2803; A61K 2039/507

(57) ABSTRACT

Use of an IL-1β binding antibody or a functional fragment thereof, especially canakinumab or a functional fragment thereof, or gevokizumab or a functional fragment thereof, and biomarkers for the treatment and/or prevention of cancer with at least partial inflammatory basis, e.g., MDS.

### TECHNICAL FIELD

The present invention relates to the use of an IL-1β binding antibody or a functional fragment thereof, for the treatment and/or prevention of cancers, e.g., cancers having at least a partial inflammatory basis.

### BACKGROUND OF THE DISCLOSURE

The majority of cancers is still incurable. There remains a continued need to develop new treatment options for cancers.

### SUMMARY OF THE DISCLOSURE

The present disclosure relates to the use of an IL-1β binding antibody or a functional fragment thereof, suitably canakinumab, suitably gevokizumab, for the treatment and/or prevention of cancers, e.g., cancers that have at least a partial inflammatory basis. Specifically, the cancer is myelodysplastic syndromes (MDS).

In another aspect, the present invention relates to a particular clinical dosage regimen for the administration of an IL-1β binding antibody or a functional fragment thereof, suitably canakinumab, suitably gevokizumab, for the treatment of MDS. In one embodiment the preferred dose of canakinumab is about 200 mg every 3 weeks or monthly, preferably subcutaneously. In one embodiment patient receives gevokizumab about 30 mg to about 120 mg per treatment every 3 weeks or monthly, preferably intravenously.

In another aspect the subject with MDS is administered with one or more anti-cancer therapeutic agent (e.g., a chemotherapeutic agent) and/or have received/will receive debulking procedures in addition to the administration of an IL-1β binding antibody or a functional fragment thereof, suitably canakinumab, suitably gevokizumab.

There are also provided methods of treating MDS in a human subject comprising administering to the subject a therapeutically effective amount of an IL-1β binding antibody or a functional fragment thereof.

Another aspect of the invention is the use of an IL-1β binding antibody or a functional fragment thereof for the preparation of a medicament for the treatment/prevention of MDS.

The present disclosure also provides a pharmaceutical composition comprising a therapeutically effective amount of an IL-1β binding antibody or a functional fragment thereof, suitably canakinumab or gevokizumab, for use in the treatment and/or prevention of MDS. In one embodiment, the pharmaceutical composition comprising a therapeutically effective amount of an IL-1β binding antibody or a functional fragment thereof, e.g., canakinumab, e.g., gevokizumab, is in the form of an auto-injector. In one embodiment about 200 mg of canakinumab is loaded in an auto-injector. In one embodiment, about 250 mg of canakinumab is loaded in an auto-injector.

### FIGURE LEGENDS

FIG. 1. In vivo model of spontaneous human breast cancer metastasis to human bone predicts a key role for IL-1β signaling in breast cancer bone metastasis. Two 0.5 cm³ pieces of human femoral bone were implanted subcutaneously into 8-week old female NOD SCID mice (n=10/group). 4 weeks later luciferase labelled MDA-MB-231-luc2-TdTomato or T47D cells were injected into the hind mammary fat pads. Each experiment was carried out 3-separate times using bone form a different patient for each repeat. Histograms showing fold change of IL-1B, IL-1R1, Caspase 1 and IL-1Rα copy number (dCT) compared with GAPDH in tumour cells grown in vivo compared with those grown in a tissue culture flask (a i); mammary tumours that metastasise compared with mammary tumours that do not metastasise (a ii); circulating tumour cells compared with tumour cells that remain in the fat pad (a iii) and bone metastases compared with the matched primary tumour (a iv). Fold change in IL-1β protein expression is shown in (b) and fold change in copy number of genes associated with EMT (E-cadherin, N-cadherin and JUP) compared with GAPDH are shown in (c). *=P<0.01, **=P<0.001, ***=P<0.0001, \hat{ }\hat{ }\hat{ }=P<0.001 compared with naïve bone.

FIG. 2. Stable transfection of breast cancer cells with IL-1B. MDA-MB-231, MCF7 and T47D breast cancer cells were stably transfected with IL-1B using a human cDNA ORF plasmid with a C-terminal GFP tag or control plasmid. a) shows pg/ng IL-1β protein from IL-1β-positive tumour cell lysates compared with scramble sequence control. b) shows pg/ml of secreted IL-1β from 10,000 IL-1β+ and control cells as measured by ELISA. Effects of IL-1B overexpression on proliferation of MDA-MB-231 and MCF7 cells are shown in (c and d) respectively. Data shown are mean+/−SEM, *=P<0.01, **=P<0.001, ***=P<0.0001 compared with scramble sequence control.

FIG. 3. Tumour derived IL-1β induces epithelial to mesenchymal transition in vitro. MDA-MB-231, MCF7 and T47D cells were stably transfected with to express high levels of IL-1B, or scramble sequence (control) to assess effects of endogenous IL-1B on parameters associated with metastasis. Increased endogenous IL-1B resulted tumour cells changing from an epithelial to mesenchymal phenotype (a). b) shows fold-change in copy number and protein expression of IL-1B, IL-1R1, E-cadherin, N-cadherin and JUP compared with GAPDH and f-catenin respectively. Ability of tumour cells to invade towards osteoblasts through Matrigel and/or 8 μM pores, are shown in (c) and capacity of cells to migrate over 24 and 48 h is shown using a wound closure assay (d). Data are shown as mean+/−SEM, *=P<0.01, **=P<0.001, ***=P<0.0001.

FIG. 4. Pharmacological blockade of IL-1β inhibits spontaneous metastasis to human bone in vivo. Female NOD-SCID mice bearing two 0.5 cm³ pieces of human femoral bone received intra-mammary injections of MDA-MB-231Luc2-TdTomato cells. One week after tumour cell injection mice were treated with 1 mg/kg/day IL-1Ra, 20 mg/kg/14-days canakinumab, or placebo (control) (n=10/group). All animals were culled 35 days following tumour cell injection. Effects on bone metastases (a) were assessed in vivo and immediately post-mortem by luciferase imaging and confirmed ex vivo on histological sections. Data are shown as numbers of photons per second emitted 2 minutes following sub-cutaneous injection of D-luciferin. Effects on numbers of tumour cells detected in the circulation are shown in (b). *=P<0.01, **=P<0.001, ***=P<0.0001.

FIG. 5. Tumour derived IL-1β promotes breast cancer bone homing in vivo. 8-week old female BALB/c nude mice were injected with control (scramble sequence) or IL-1β overexpressing MDA-MB-231-IL-1β+ cells via the lateral tail vein. Tumour growth in bone and lung were measured in vivo by GFP imaging and findings confirmed ex vivo on histological sections. a) shows tumour growth in bone; b) shows representative μCT images of tumour bearing tibiae and the graph shows bone volume (BV)/tissue volume (TV) ratio indicating effects on tumour induced bone destruction; c) shows numbers and size of tumours detected in lungs from each of the cell lines. *=P<0.01, **=P<0.001, ***=P<0.0001. (B=bone, T=tumour, L=lung)

FIG. 6. Tumour cell-bone cell interactions stimulate IL-1β production cell proliferation. MDA-MB-231 or T47D human breast cancer cell lines were cultured alone or in combination with live human bone, HS5 bone marrow cells or OB1 primary osteoblasts. a) shows the effects of culturing MDA-MB-231 or T47D cells in live human bone discs on IL-1β concentrations secreted into the media. The effect of co-culturing MDA-MB-231 or T47D cells with HS5 bone cells on IL-1β derived from the individual cell types following cell sorting and the proliferation of these cells are shown in b) and c). Effects of co-culturing MDA-MB-231 or T47D cells with OB1 (osteoblast) cells on proliferation are shown in d). Data are shown as mean+/−SEM, *=P<0.01, **=P<0.001, ***=P<0.0001.

FIG. 7. IL-1β in the bone microenvironment stimulates expansion of the bone metastatic niche. Effects of adding 40 pg/ml or 5 ng/ml recombinant IL-1β to MDA-MB-231 or T47D breast cancer cells is shown in (a) and effects on adding 20 pg/ml, 40 pg/ml or 5 ng/ml IL-1B on proliferation of HS5, bone marrow, or OB1, osteoblasts, are shown in b) and c) respectively. (d) IL-1 driven alterations to the bone vasculature was measured following CD34 staining in the trabecular region of the tibiae from 10-12-week old female IL-1R1 knockout mice. (e) BALB/c nude mice treated with 1 mg/ml/day IL-1Rα for 31 days and (f) C57BL/6 mice treated with 10 μM canakinumab for 4-96 h. Data are shown as mean+/−SEM, *=P<0.01, **=P<0.001, ***=P<0.0001.

FIG. 8. Suppression of IL-1 signalling affects bone integrity and vasculature. Tibiae and serum from mice that do not express IL-1R1 (IL-1R1 KO), BALB/c nude mice treated daily with 1 mg/kg per day of IL-1R antagonist for 21 and 31 days and C57BL/6 mice treated with 10 mg/kg of canakinumab (Ilaris) of 0-96 h were analysed for bone integrity by μCT and vasculature using ELISA for Endothelin 1 and pan VEGF. a) shows the effects of IL-1R1 KO; b) effects of Anakinra and c) effects of canakinumab on bone volume compared with tissue volume (i), concentration of Endothelin 1 (ii) and concentrations of VEGF secreted into the serum. Data shown are mean+/−SEM, *=P<0.01, **=P<0.001, ***=P<0.0001 compared with control.

FIG. 9. Tumour derived IL-1β predicts future recurrence and bone relapse in patients with stage II and III breast cancer. ˜1300 primary breast cancer samples from patients with stage II and III breast cancer with no evidence of metastasis were stained for 17 kD active IL-1β. Tumours were scored for IL-1β in the tumour cell population. Data shown are Kaplan Meyer curves representing the correlation between tumour derived IL-1β and subsequent recurrence a) at any site or b) in bone over a 10-year time period.

FIG. 10. Simulation of canakinumab PK profile and hsCRP profile. a) shows canakinumab concentration time profiles. Solid line and band: median of individual simulated concentrations with 2.5-97.5% prediction interval (300 mg Q12W (bottom line), 200 mg Q3W (middle line), and 300 mg Q4W (top line)). b) shows the proportion of month 3 hsCRP being below the cut point of 1.8 mg/L for three different populations: all CANTOS patients (scenario 1), confirmed lung cancer patients (scenario 2), and advanced lung cancer patients (scenario 3) and three different dose regimens. c) is similar to b) with the cut point being 2 mg/L. d) shows the median hsCRP concentration over time for three different doses. e) shows the percent reduction from baseline hsCRP after a single dose.

FIG. 11. Gene expression analysis by RNA sequencing in colorectal cancer patients receiving PDR001 in combination with canakinumab, PDR001 in combination with everolimus and PDR001 in combination with others. In the heatmap figure, each row represents the RNA levels for the labelled gene. Patient samples are delineated by the vertical lines, with the screening (pre-treatment) sample in the left column, and the cycle 3 (on-treatment) sample in the right column. The RNA levels are row-standardized for each gene, with black denoting samples with higher RNA levels and white denoting samples with lower RNA levels. Neutrophil-specific genes FCGR3B, CXCR2, FFAR2, OSM, and G0S2 are boxed.

FIG. 12. Clinical data after gevokizumab treatment (panel a) and its extrapolation to higher doses (panels b, c, and d). Adjusted percent change from baseline in hsCRP in patients in a). The hsCRP exposure-response relationship is shown in b) for six different hsCRP base line concentrations. The simulation of two different doses of gevokizumab is shown in b) and c).

FIG. 13. Effect of anit-IL-1beta treatment in two mouse models of cancer. a), b), and c) show data from the MC38 mouse model, and d) and e) show data from the LL2 mouse model.

FIG. 14. Efficacy of canakinumab in combination with pembrolizumab in inhibiting tumor growth.

FIG. 15. Preclinical data on the efficacy of canakinumab in combination with docetaxel in the treatment of cancer.

FIG. 16. Mice were implanted with 4T1 cells sc and treated with the indicated treatments on days 8 and 15 post tumor implant. There were 10 mice in each group.

FIG. 17. Neutrophils (top) and monocytes (bottom) in 4T1 tumors 5 days after a single dose of docetaxel, 01BSUR, or the combination of docetaxel and 01BSUR.

FIG. 18. Granulocytic (top) and monocytic (bottom) MDSC in 4T1 tumors 5 days after a single dose of docetaxel, 01BSUR, or the combination of docetaxel and 01BSUR.

FIG. 19. TIM-3+CD4+(top) and CD8+(bottom) T cells in 4T1 tumors 4 days after a second dose of docetaxel, 01BSUR, or the combination of docetaxel and 01BSUR.

FIG. 20. TIM-3 expressing Tregs in 4T1 tumors 4 days after a second dose of docetaxel, 01BSUR, or the combination of docetaxel and 01BSUR.

FIG. 21. Clinical efficacy of canakinumab as compared to placebo for incident anemia according to subgroups based on baseline clinical characteristics. Data are shown as hazard ratios for combined canakinumab doses (50 mg, 150 mg, and 300 mg) as compared to placebo.

FIG. 22. Incidence of anemia in the placebo and the canakinumab groups at ≥65 years of age or <65 years of age.

### DETAILED DESCRIPTION OF THE DISCLOSURE

Many malignancies arise in areas of chronic inflammation and inadequate resolution of inflammation is hypothesized to play a major role in tumor invasion, progression, and metastases (Voronov E, et al, PNAS 2003).

There are a number of observations showing that IL-1β plays a role in MDS. Inflammation is widely described in MDS (Barreyro et al., Blood. 2018), and in particular the NLRP3 inflammasome has been shown to function as a driver of myelodysplastic syndromes phenotypes, which leads to the generation of IL-1β as well as pyroptotic cell death in MDS hematopoietic stem and progenitor cells (Basiorka et al., Blood. 2016; 128(25):2960-2975). Changes in the IL-1β gene (single nucleotide polymorphisms, SNPs) have been found to be associated with increased susceptibility to myelodysplastic syndromes, and patients with IL-1β polymorphisms had lower haemoglobin than those without (Yin et al., Life Sci. 2016; 165:109-112). Further, IL-1β has been implicated in the suppression of transcription and cellular elaboration of erythropoietin (Cluzeau et al., Haematologica. 2017; 102(12): 2015-2020). Elevated levels of IL-1β are capable of blocking erythropoietin's proliferative effects on erythroid progenitor cells in vitro (Schooley et al. 1987) and chronic exposure of hematopoietic stem cells to elevated IL-1β promoted myeloid differentiation, suppressed erythroid differentiation and led to hematopoietic stem cell exhaustion in vivo (Pietras et al. 2016). Also, IL-1β (along with TNFα) have been identified as myelosuppressive cytokines that are secreted by bone marrow cells in a p38 MAPK-dependent manner, leading to CD34+ stem cell apoptosis (Navas et al., Leuk Lymphoma. 2008; 49(10):1963-75).

As reported in Ridker et al. (Lancet, 2017), a randomised, double-blind, placebo-controlled trial of canakinumab in 10061 patients with atherosclerosis who had had a myocardial infarction, were free of previously diagnosed cancer, and had concentrations of high-sensitivity C-reactive protein (hsCRP) of 2 mg/L or greater was completed in June, 2017 (CANTOS trial). To assess dose-response effects, patients were randomly assigned by computer-generated codes to three canakinumab doses (50 mg, 150 mg, and 300 mg, subcutaneously every 3 months) or placebo.

Baseline concentrations of hsCRP (median 6·0 mg/L vs 4·2 mg/L; p<0·0001) and interleukin 6 (3·2 vs 2·6 ng/L; p<0·0001) were significantly higher among participants subsequently diagnosed with lung cancer than among those not diagnosed with cancer. During median follow-up of 3·7 years, compared with placebo, canakinumab was associated with dose-dependent reductions in concentrations of hsCRP of 26-41% and of interleukin 6 of 25-43% (p<0·0001 for all comparisons). Total cancer mortality (n=196) was significantly lower in the pooled canakinumab group than in the placebo group (p=0·0007 for trend across groups), but was significantly lower than placebo only in the 300 mg group individually (hazard ratio [HR]0·49 [95% CI 0·31-0·75]; p=0·0009). Incident lung cancer (n=129) was significantly less frequent in the 150 mg (HR 0·61 [95% CI 0·39-0·97]; p=0·034) and 300 mg groups (HR 0·33 [95% CI 0·18-0·59]; p<0·0001; p<0·0001 for trend across groups). Lung cancer mortality was significantly less common in the canakinumab 300 mg group than in the placebo group (HR 0·23 [95% CI 0·10-0·54]; p=0·0002) and in the pooled canakinumab population than in the placebo group (p=0·0002 for trend across groups).

Biomarker analysis of patients of non-lung cancers from the CANTOS trial, especially of the GI/GU cancers, has revealed that they have elevated baseline hsCRP level and IL-6 level. In addition, GI/GU cancer patients with higher baseline level of hsCRP and IL-6 seem to have a shorter time to cancer diagnosis than patients having lower baseline level (EXAMPLE 11), suggesting the likelihood of the involvement of IL-1β mediated inflammation in broader cancer indications, besides lung cancer, which warranties targeting IL-1β in the treatment of those cancers. In addition hsCRP level and IL-6 level in GI/GU patients were reduced in the range comparable to other patients in the CANTOS trial treatment group, suggesting inhibition of IL-1β signaling in those patients. Inhibition of IL-1β alone or preferably in combination with other anti-cancer agents could result in clinical benefit in treating cancer, e.g., cancer having at least partial inflammatory basis, as further supported by data presented in EXAMPLES.

### Cancers, e.g., Cancers Having at Least a Partial Inflammatory Basis

Thus in one aspect, the present invention provides the use of an IL-1β binding antibody or a functional fragment thereof (for reason of simplicity, the term “an IL-1β binding antibody or a functional fragment thereof” is sometimes referred as “DRUG of the invention” in this application, which should be understood as identical term), suitably canakinumab or a functional fragment thereof (included in “DRUG of the invention”), gevokizumab or a functional fragment thereof (included in “DRUG of the invention”), for the treatment and/or prevention of MDS.

Advanced studies in delineating interaction between the tumor and the tumor microenvironment have revealed that chronic inflammation can promote tumor development, and the tumor fuels inflammation to facilitate tumor progression and metastasis. The inflammatory microenvironment with cellular and non-cellular secreted factors provides a sanctuary for tumor progression by inducing angiogenesis; recruiting tumor promoting, immune suppressive cells; and inhibiting immune effector cell mediated anti-tumor immune response. One of the major inflammatory pathways supporting tumor development and progression is IL-1β, a pro-inflammatory cytokine produced by tumors and tumor associated immune suppressive cells including neutrophils and macrophages in tumor microenvironment.

Accordingly, the present disclosure provides a method of treating cancer using an IL-1β binding antibody or a functional fragment thereof, wherein such IL-1β binding antibodies or functional fragments thereof can reduce inflammation and/or improve the tumor microenvironment, e.g., they can inhibit IL-1β mediated inflammation and IL-1β mediated immune suppression in the tumor microenvironment. An example of using an IL-1β binding antibody in modulating the tumor microenvironment is shown in Example 6 herein. In some embodiments, an IL-1β binding antibody or a functional fragment thereof is used alone as a monotherapy. In some embodiments, an IL-1β binding antibody or a functional fragment thereof is used in combination with another therapy, such as with a check point inhibitor and/or with one or more chemotherapeutic agents. As discussed herein, inflammation can promote tumor development, an IL-1β binding antibody or a functional fragment thereof, either alone or in combination with another therapy, can be used to treat any cancer that can benefit from reduced IL-1β mediated inflammation and/or improved tumor environment. An inflammation component is universally present, albeit to different degrees, in cancer development.

The meaning of “cancers that have at least a partial inflammatory basis” or “cancer having at least a partial inflammatory basis” is well known in the art and as used herein refers to any cancer in which IL-1β mediated inflammatory responses contribute to tumor development and/or propagation, including but not necessarily limited to metastasis. Such cancer generally has concomitant inflammation activated or mediated in part through activation of the Nod-like receptor protein 3 (NLRP3) inflammasome with consequent local production of interleukin-10. In a patient with such cancer, the expression, or even the overexpression of IL-1β can be generally detected, commonly at the site of the tumor, especially in the surrounding tissue of the tumor, in comparison to normal tissue. The expression of IL-1β can be detected by routine methods known in the art, such as immunostaining, ELISA based assays, ISH, RNA sequencing or RT-PCR in the tumor as well as in serum/plasma. The expression or higher expression of IL-1β can be concluded, for example, against a negative control, usually normal tissue at the same site or can be concluded if higher than normal level of IL-1β is present in the serum/plasma of a heathy person (reference value). Simultaneously or alternatively, a patient with such cancer generally has chronic inflammation, which is manifested, typically, by higher than normal levels of hsCRP (or CRP), IL-6 or TNFα, preferably by hsCRP or IL-6, preferably by IL-6. This is because IL-6 is immediately downstream of IL-1β. HsCRP is further downstream and can be influenced by other factors. Cancers, particularly cancers that have at least a partial inflammatory basis, include MDS. Cancers also include cancers that may not express IL-1β initially, and only start expressing IL-1β after treatment of such cancer, e.g., including treatment with a chemotherapeutic agent, e.g., as described herein, which contributes to the expression of IL-1β in the tumor and/or tumor microenvironment. In some embodiments, the methods and use comprise treating a patient whose cancer is relapsed or recurring after treatment with such agent. In other embodiments, the agent is associated with IL-1β expression and the IL-1β antibody or functional fragment thereof is given in combination with such agent.

Inhibition of IL-1β resulted in reduced inflammation status, including but not limited to reduced hsCRP or IL-6 level. Thus the effect of the present invention in cancer patients can be measured by reduced inflammation status, including but not limited to reduced hsCRP or IL-6 level.

The term “cancers that have at least a partial inflammatory basis” or “cancer having at least a partial inflammatory basis” also includes cancers that benefit from the treatment of an IL-1β binding antibody or a functional fragment thereof. As inflammation in general contributes to tumor growth at already an early stage, administration of IL-1β binding antibody or a functional fragment thereof (canakinumab or gevokizumab) could potentially stop tumor growth effectively at the early stage or delay tumor progression effectively at the early stage, even though the inflammation status, such as expression or overexpression IL-1β, or the elevated level of CRP or hsCRP, IL-6 or TNFα, is still not apparent or measurable. However, patients having early stage cancers can still benefit from the treatment with an IL-1β binding antibody or a functional fragment thereof, which can be shown in clinical trials. The clinical benefit can be measured by, including but not limited to, disease-free survival (DFS), progression-free survival (PFS), overall response rate (ORR), disease control rate (DCR), duration of response (DOR) and overall survival (OS), preferably in a clinical trial setting, against a proper control group, for example against the effects achieved by standard of care (SoC) drugs, either by addition on top of SoC or without SoC. If a patient treated with the DRUG of the invention has shown any improvement in one or more of the above parameters in comparison to the control, the patient is considered to have benefited from the treatment according to the present invention.

Available techniques known to the skilled person in the art allow detection and quantification of IL-1β in tissue as well as in serum/plasma, particularly when the IL-1β is expressed at a higher than normal level. For example, using the R&D Systems high sensitivity IL-1β ELISA kit, IL-1β cannot be detected in the majority of healthy donor serum samples, as

### Sample Values

Serum/Plasma—Samples from apparently healthy volunteers were evaluated for the presence of human IL-1β in this assay. No medical histories were available for the donors used in this study.

| | Mean of Detectable | | Range |
|---|---|---|---|
| Sample Type | (pg/mL) | % Detectable | (pg/mL) |
| Serum (n = 50) | 0.357 | 10 | ND-0.606 |
| EDTA plasma (n = 50) | 0.292 | 12 | ND-0.580 |
| Heparin plasma (n = 50) | 0.448 | 14 | ND-1.08 |

ND = Non-detectable

shown in the following Table.

Thus in a healthy person the IL-1β level is barely detectable or just above the detection limit according to this test with the high sensitivity R&D® IL-1β ELISA kit. It is expected that a patient with cancer having at least partial inflammatory basis in general has higher than normal levels of IL-1β and that the levels of IL-1β can be detected by the same kit. Taking the IL-1β expression level in a healthy person as the normal level (reference level), the term “higher than normal level of IL-1β” means an IL-1β level that is higher than the reference level. Normally at least about 2 fold, at least about 5 fold, at least about 10 fold, at least about 15 fold, or at least about 20 fold of the reference level is considered as higher than normal level. Alternatively taking the IL-1β expression level in a healthy person as the normal level (reference level), the term “higher than normal level of IL-1β” means an IL-1β level that is higher than the reference level, normally higher than 0.8 pg/ml, higher than 1 pg/ml, higher than 1.3 pg/ml, higher than 1.5 pg/ml, higher than 2 pg/ml, higher than 3 pg/ml, as determined preferably by the R&D kit mentioned above. Blocking the IL-1β pathway normally triggers the compensating mechanism leading to more production of IL-1β. Thus the term “higher than normal level of IL-1” also means and includes the level of IL-1β either post, or more preferably, prior to the administration of an IL-1β binding antibody or a fragment thereof. Treatment of cancer with agents other than IL-1β inhibitors, such as some chemotherapeutic agents, can result in production of IL-1β in the tumor microenvironment. Thus the term “higher than normal level of IL-1β” also refers to the level of IL-1β either prior to or post the administration of such an agent.

When using staining, such as immunostaining, to detect IL-1β expression in a tissue preparation, the term “higher than normal level of IL-1” means to that the staining signal generated by a specific IL-1β protein or IL-1β RNA detecting molecule is distinguishably stronger than the staining signal of the surrounding tissue not expressing IL-1β.

Available techniques known to the skilled person in the art allow detection and quantification of IL-6 in tissue as well as in serum/plasma, particularly when the IL-6 is expressed to a higher than normal level. For example, using the R&D Systems (www.RnDsystems.com) “high quantikine HS ELISA, human IL-6 Immnunoassay”, IL-6 can be detected in majority of healthy donor serum samples, as shown in the following Table.

### Sample Values

Samples from apparently healthy volunteers were evaluated for the presence of human IL-6 in this assay. No medical histories were available for the donors used in this study.

| | Mean of Detectable | | Range |
|---|---|---|---|
| Sample Type | (pg/mL) | % Detectable | (pg/mL) |
| Serum (n = 52) | 1.77 | 100 | 0.447-9.96 |
| EDTA plasma (n = 35) | 1.49 | 100 | 0.428-8.87 |
| Citrate plasma (n = 16) | 1.57 | 100 | 0.435-9.57 |
| Urine (n = 14) | 1.67 | 93 | ND-6.76 |

ND = Non-detectable

It is expected that in a patient with cancer having at least partial inflammatory basis in general has higher than normal level of IL-6 and can be detected by the same kit. Taking the IL-6 expression level in a healthy person as the normal level (reference level), the term “higher than normal level of IL-6” means an IL-6 level that is higher than the reference level, normally higher than 1.9 pg/ml, higher than 2 pg/ml, higher than 2.2 pg/ml, higher than 2.5 pg/ml, higher than 2.7 pg/ml, higher than 3 pg/ml, higher than 3.5 pg/ml, or higher than 4 pg/ml, as determined preferably by the R&D kit mentioned above. Blocking the IL-1β pathway normally triggers the compensating mechanism leading to more production of IL-1β. Thus the term “higher than normal level of IL-6” also means and includes the level of IL-6 either post, or more preferably, prior to the administration of an IL-1β binding antibody or a fragment thereof. Treatment of cancer with agents other than IL-1β inhibitors, such as some chemotherapeutic agents, can result in production of IL-1β in the tumor microenvironment. Thus the term “higher than normal level of IL-6” also refers to the level of IL-6 either prior to or post the administration of such an agent.

When using staining, such as immunostaining, to detect IL-6 expression in a tissue preparation, the term “higher than normal level of IL-6” means that the staining signal generated by specific IL-6 protein or IL-6 RNA detecting molecule is distinguishably stronger than staining signal of the surrounding tissue not expressing IL-6.

As used herein, the terms “treat”, “treatment” and “treating” refer to the reduction or amelioration of the progression, severity, and/or duration of a disorder, e.g., a proliferative disorder, or the amelioration of one or more symptoms, suitably of one or more discernible symptoms, of the disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of MDS factors in the patient (using International prognostic scoring system (IPSS and revised IPSS-R) and/or the WHO prognostic scoring system (WPSS) for quantification) or the reduction or stabilization of a cancerous cell count. As far as cancers as discussed here, taking MDS as an example, the term treatment refers to at least one of the following: alleviating one or more symptoms of MDS, delaying progression of MDS, improvement of MDS factors in the patient, stabilization of MDS factors in the patient, prolonging overall survival, prolonging progression free survival, preventing or delaying MDS tumor metastasis, preventing or delaying the progression of MDS to secondary acute myeloid leukemia, reducing (such as eradiating) pre-existing MDS metastases, reducing incidence or burden of pre-existing MDS metastasis, or preventing recurrence of MDS.

### IL-1β Inhibitors, Especially IL-1β Binding Antibody or a Fragment Thereof

As used herein, IL-1β inhibitors include but are not limited to, canakinumab or a functional fragment thereof, gevokizumab or a functional fragment thereof, Anakinra, diacerein, Rilonacept, IL-1 Affibody (SOBI 006, Z-FC (Swedish Orphan Biovitrum/Affibody)) and Lutikizumab (ABT-981) (Abbott), CDP-484 (Celltech), LY-2189102 (Lilly).

In one embodiment of any use or method of the invention, said IL-1β binding antibody is canakinumab. Canakinumab (ACZ885) is a high-affinity, fully human monoclonal antibody of the IgG1/k to interleukin-1l, developed for the treatment of IL-1β driven inflammatory diseases. It is designed to bind to human IL-1β and thus blocks the interaction of this cytokine with its receptors.

In other embodiments of any use or method of the invention, said IL-1β binding antibody is gevokizumab. Gevokizumab (XOMA-052) is a high-affinity, humanized monoclonal antibody of the IgG2 isotype to interleukin-1l, developed for the treatment of IL-1β driven inflammatory diseases. Gevokizumab modulates IL-1β binding to its signaling receptor.

In one embodiment said IL-1β binding antibody is LY-2189102, which is a humanised interleukin-1 beta (IL-1β) monoclonal antibody.

In one embodiment said IL-1β binding antibody or a functional fragment thereof is CDP-484 (Celltech), which is an antibody fragment blocking IL-1β.

In one embodiment said IL-1β binding antibody or a functional fragment thereof is IL-1 Affibody (SOBI 006, Z-FC (Swedish Orphan Biovitrum/Affibody)).

An antibody, as used herein, refers to an antibody having the natural biological form of an antibody. Such an antibody is a glycoprotein and consists of four polypeptides—two identical heavy chains and two identical light chains, joined to form a “Y”-shaped molecule. Each heavy chain is comprised of a heavy chain variable region (VH) and a heavy chain constant region. The heavy chain constant region is comprised of three or four constant domains (CH1, CH2, CH3, and CH4, depending on the antibody class or isotype). Each light chain is comprised of a light chain variable region (VL) and a light chain constant region, which has one domain, CL. Papain, a proteolytic enzyme, splits the “Y” shape into three separate molecules, two so called “Fab” fragments (Fab=fragment antigen binding), and one so called “Fc” fragment (Fc=fragment crystallizable). A Fab fragment consists of the entire light chain and part of the heavy chain. The VL and VH regions are located at the tips of the “Y”-shaped antibody molecule. The VL and VH each have three complementarity-determining regions (CDRs).

By “IL-1β binding antibody” is meant any antibody capable of binding to the IL-1β specifically and consequently inhibiting or modulating the binding of IL-1β to its receptor and further consequently inhibiting IL-1β function. Preferably an IL-1β binding antibody does not bind to IL-1α.

Preferably an IL-1β binding antibody includes:

(1) An antibody comprising three VL CDRs having the amino acid sequences RASQSIGSSLH (SEQ ID NO: 1), ASQSFS (SEQ ID NO: 2), and HQSSSLP (SEQ ID NO: 3) and three VH CDRs having the amino acid sequences VYGMN (SEQ ID NO: 5), IIWYDGDNQYYADSVKG (SEQ ID NO: 6), and DLRTGP (SEQ ID NO: 7);

(2) An antibody comprising three VL CDRs having the amino acid sequences RASQDISNYLS (SEQ ID NO: 9), YTSKLHS (SEQ ID NO: 10), and LQGKMLPWT (SEQ ID NO: 11), and three VH CDRs having the amino acid sequences TSGMGVG (SEQ ID NO: 13), HIWWDGDESYNPSLK (SEQ ID NO: 14), and NRYDPPWFVD (SEQ ID NO: 15); and

(3) An antibody comprising the six CDRs as described in either (1) or (2), wherein one or more of the CDR sequences, preferably at most two of the CDRs, preferably only one of the CDRs, differ by one amino acid from the corresponding sequences described in either (1) or (2), respectively.

Preferably an IL-1β binding antibody includes:

(1) An antibody comprising three VL CDRs having the amino acid sequences RASQSIGSSLH (SEQ ID NO: 1), ASQSFS (SEQ ID NO: 2), and HQSSSLP (SEQ ID NO: 3) and comprising the VH having the amino acid sequence specified in SEQ ID NO: 8;

(2) An antibody comprising the VL having the amino acid sequence specified in SEQ ID NO: 4 and comprising three VH CDRs having the amino acid sequences VYGMN (SEQ ID NO: 5), IIWYDGDNQYYADSVKG (SEQ ID NO: 6), and DLRTGP (SEQ ID NO: 7);

(3) An antibody comprising three VL CDRs having the amino acid sequences RASQDISNYLS (SEQ ID NO: 9), YTSKLHS (SEQ ID NO: 10), and LQGKMLPWT (SEQ ID NO: 11), and comprising the VH having the amino acid sequences specified in SEQ ID NO: 16;

(4) An antibody comprising the VL having the amino acid specified in SEQ ID NO: 12, and comprising three VH CDRs having the amino acid sequences TSGMGVG (SEQ ID NO: 13), HIWWDGDESYNPSLK (SEQ ID NO: 14), and NRYDPPWFVD (SEQ ID NO: 15);

(5) An antibody comprising three VL CDRs and the VH sequence as described in either (1) or (3), wherein one or more of the VL CDR sequences, preferably at most two of the CDRs, preferably only one of the CDRs, differ by one amino acid from the corresponding sequences described in (1) or (3), respectively, and wherein the VH sequence is at least 90% identical to the corresponding sequence described in (1) or (3), respectively; and

(6) An antibody comprising the VL sequence and three VH CDRs as described in either (2) or (4), wherein the VL sequence is at least 90% identical to the corresponding sequence described in (2) or (4), respectively, and wherein one or more of the VH CDR sequences, preferably at most two of the CDRs, preferably only one of the CDRs, differ by one amino acid from the corresponding sequences described in (2) or (4), respectively.

Preferably an IL-1β binding antibody includes:

(1) An antibody comprising the VL having the amino acid sequence specified in SEQ ID NO: 4 and comprising the VH having the amino acid sequence specified in SEQ ID NO: 8;

(2) An antibody comprising the VL having the amino acid specified in SEQ ID NO: 12, and comprising the VH having the amino acid sequences specified in SEQ ID NO: 16; and

(3) An antibody described in either (1) or (2), wherein the constant region of the heavy chain, the constant region of the light chain or both has been changed to a different isotype as compared to canakinumab or gevokizumab.

Preferably an IL-1β binding antibody includes:

(1) Canakinumab (SEQ ID NO:17 and 18); and

(2) Gevokizumab (SEQ ID NO:19 and 20).

An IL-1β binding antibody as defined above has substantially identical or identical CDR sequences as those of canakinumab or gevokizumab. It thus binds to the same epitope on IL-1β and has similar binding affinity as canakinumab or gevokizumab. The clinical relevant doses and dosing regimens that have been established for canakinumab or gevokizumab as therapeutically efficacious in the treatment of cancer, especially cancer having at least partial inflammatory basis, would be applicable to other IL-1β binding antibodies.

Additionally or alternatively, an IL-1β antibody refers to an antibody that is capable of binding to IL-1β specifically with affinity in the similar range as canakinumab or gevokizumab. The Kd for canakinumab in WO2007/050607 is referenced with 30.5 pM, whereas the Kd for gevokizumab is 0.3 pM. Thus affinity in the similar range refers to between about 0.05 pM to 300 pM, preferably 0.1 pM to 100 pM. Although both binding to IL-1β, canakinumab directly inhibits the binding to IL-1 receptor, whereas gevokizumab is an allosteric inhibitor. It does not prevent IL-1β from binding to the receptor but prevent receptor activation. Preferably an IL-1β antibody has the binding affinity in the similar range as canakinumab, preferably in the range of 1 pM to 300 pM, preferably in the range of 10 pM to 100 pM, wherein preferably said antibody directly inhibits binding. Preferably an IL-1β antibody has the binding affinity in the similar range as gevokizumab, preferably in the range of 0.05 pM to 3 pM, preferably in the range of 0.1 pM to 1 pM, wherein preferably said antibody is an allosteric inhibitor.

As used herein, the term “functional fragment” of an antibody as used herein, refers to portions or fragments of an antibody that retain the ability to specifically bind to an antigen (e.g., IL-1β). Examples of binding fragments encompassed within the term “functional fragment” of an antibody include single chain Fv (scFv), a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989), which consists of a VH domain; and an isolated complementarity determining region (CDR); and one or more CDRs arranged on peptide scaffolds that can be smaller, larger, or fold differently to a typical antibody.

The term “functional fragment” might also refer to one of the following:
bispecific single chain Fv dimers (PCT/US92/09965)
“diabodies” or “triabodies”, multivalent or multispecific fragments constructed by gene fusion (Tomlinson I & Hollinger P (2000) Methods Enzymol. 326: 461-79; WO94113804; Holliger P et al., (1993) Proc. Natl. Acad. Sci. USA, 90: 6444-48)
scFv genetically fused to the same or a different antibody (Coloma M J & Morrison S L (1997) Nature Biotechnology, 15(2): 159-163)
scFv, diabody or domain antibody fused to an Fc region
scFv fused to the same or a different antibody
Fv, scFv or diabody molecules may be stabilized by the incorporation of disulphide bridges linking the VH and VL domains (Reiter, Y. et al, (1996) Nature Biotech, 14, 1239-1245).
Minibodies comprising a scFv joined to a CH3 domain may also be made (Hu, S. et al, (1996) Cancer Res., 56, 3055-3061).
Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group

Typically and preferably an functional fragment of an IL-1β binding antibody is a portion or a fragment of an “IL-1β binding antibody” as defined above.

### Dosing Regimens of the Present Invention

If an IL-1β inhibitor, such as an IL-1β antibody or a functional fragment thereof, is administered in a dose range that can effectively reduce hsCRP level in a patient with cancer having at least partial inflammatory basis, treatment effect of said cancer can possibly be achieved. The dose range of a particular IL-1β inhibitor, preferably an IL-1β antibody or a functional fragment thereof, that can effectively reduce hsCRP levels is known or can be tested in a clinical setting.

Thus in one embodiment, the present invention comprises administering the IL-1β binding antibody or a functional fragment thereof to a patient with cancer, e.g., cancer that has at least a partial inflammatory basis, in the range of about 20 mg to about 400 mg per treatment, preferably in the range of about 30 mg to about 400 mg per treatment, preferably in the range of about 30 mg to about 200 mg per treatment, preferably in the range of about 60 mg to about 200 mg per treatment. In one embodiment the patient receives each treatment about every two weeks, about every three weeks, about every four weeks (monthly), about every 6 weeks, about bimonthly (about every 2 months), about every nine weeks or about quarterly (about every 3 months). In one embodiment patient receives each treatment about every 3 weeks. In one embodiment patient receives each treatment about every 4 weeks. The term “per treatment”, as used in this application and particularly in this context, should be understood as the total amount of drug received per hospital visit or per self administration or per administration helped by a health care giver. Normally and preferably the total amount of drug received per treatment is administered to a patient is within about 2 hours, preferably within about one hour, or within about half hour. In one preferred embodiment the term “per treatment” is understood as the drug is administered with one injection, preferably in one dosage.

In practice sometimes the time interval cannot be strictly kept due to the limitation of the availability of doctor, patient or the drug/facility. Thus the time interval can slightly vary, normally between about 5 days, about 4 days, about 3 days, about 2 days or preferably about 1 day.

Sometimes itis desirable to quickly reduce inflammation. IL-1β auto-induction has been shown in human mononuclear blood, human vascular endothelial, and vascular smooth muscle cells in vitro and in rabbits in vivo where IL-1 has been shown to induce its own gene expression and the circulating IL-1β level (Dinarello et al. 1987, Warner et al. 1987a, and Warner et al. 1987b).

This induction period over about 2 weeks by administration of a first dose followed by a second dose about two weeks after administration of the first dose is to assure that auto-induction of IL-1β pathway is adequately inhibited at initiation of treatment. The complete suppression of IL-1β related gene expression achieved with this early high dose administration, coupled with the continuous canakinumab treatment effect, which has been proven to last the entire quarterly dosing period used in CANTOS, is to minimize the potential for IL-1β rebound. In addition, data in the setting of acute inflammation suggests that higher initial doses of canakinumab, which can be achieved through induction, are safe and provide an opportunity to ameliorate concerns regarding to potential auto-induction of IL-1β and achieve greater early suppression of IL-1β related gene expression.

Thus in one embodiment, the present invention, while keeping the above described dosing schedules, especially envisages the second administration of DRUG of the invention is about one week or at most about two weeks, preferably about two weeks, apart from the first administration. Then the third and the further administration will following the schedule of about every 2 weeks, about every 3 weeks, about every 4 weeks (monthly), about every 6 weeks, about bimonthly (about every 2 months), about every 9 weeks, or about quarterly (about every 3 months).

In one embodiment, the IL-1β binding antibody is canakinumab, wherein canakinumab is administered to a patient with cancer, e.g., cancer that has at least a partial inflammatory basis, in the range of about 100 mg to about 400 mg, preferably about 200 mg per treatment. In one embodiment the patient receives each treatment about every 2 weeks, about every 3 weeks, about every 4 weeks (about monthly), about every 6 weeks, about bimonthly (about every 2 months), about every 9 weeks, or about quarterly (about every 3 months). In one embodiment the patient receives canakinumab about monthly or about every three weeks. In one embodiment the preferred dose of canakinumab for patient is about 200 mg every 3 weeks. In one embodiment the preferred dose of canakinumab is about 200 mg monthly. When safety concerns raise, the dose can be down-titrated, preferably by increasing the dosing interval, preferably by doubling or tripling the dosing interval. For example the about 200 mg about monthly or about every 3 weeks regimen can be changed to about every 2 months or about every 6 weeks respectively or about every 3 months or about every 9 weeks, respectively. In an alternative embodiment the patient receives canakinumab at a dose of about 200 mg about every two months or about every 6 weeks in the down-titration phase or in the maintenance phase, independent from any safety issue or throughout the treatment phase. In an alternative embodiment the patient receives canakinumab at a dose of about 200 mg about every 3 months or about every 9 weeks in the down-titration phase or in the maintenance phase independent from any safety issue or throughout the treatment phase. In an alternative embodiment the patient receives canakinumab at a dose of about 150 mg, about 250 mg, or about 300 mg. In an alternative embodiment the patient receives canakinumab at a dose of about 150 mg about every 4 weeks. In an alternative embodiment the patient receives canakinumab at a dose of about 250 mg about every 4 weeks. In an alternative embodiment the patient receives canakinumab at a dose of about 300 mg about every 4 weeks.

Suitably, the above dose and dosing apply to the use of a functional fragment of canakinumab according to the present invention.

Canakinumab or a functional fragment thereof can be administered intravenously or subcutaneously, preferably subcutaneously.

The dosing regimens disclosed herein are applicable in each and every canakinumab related embodiment disclosed in this application, including but not limited to monotherapy or in combination with one or more anti-cancer therapeutic agents, used in adjuvant setting or in the first line, 2^{nd} line or 3^{rd} line treatment.

In one embodiment, the present invention comprises administering gevokizumab to a patient with cancer, e.g., cancer that has at least a partial inflammatory basis, in the range of about 20 mg to about 240 mg per treatment, preferably in the range of about 20 mg to about 180 mg, preferably in the range of about 30 mg to about 120 mg, perferably about 30 mg to about 60 mg, preferably about 60 mg to about 120 mg per treatment. In one embodiment patient recieves about 30 mg to about 120 mg per treatment. In one embodiment patient recieves about 30 mg to about 60 mg per treatment. In one embodiment patient recieves about 30 mg, about 60 mg, about 90 mg, about 120 mg, or about 180 mg per treatment. In one embodiment the patient receives each treatment about every 2 weeks, about every 3 weeks, about monthly (about every 4 weeks), about every 6 weeks, about bimonthly (about every 2 months), about every 9 weeks or about quarterly (about every 3 months). In one embodiment the patient receives each treatment about every 3 weeks. In one embodiment the patient receives each treatment about every 4 weeks.

When safety concerns raise, the dose can be down-titrated, preferably by increasing the dosing interval, preferably by doubling or tripling the dosing interval. For example the about 60 mg about monthly or about every 3 weeks regimen can be doubled to about every 2 months or about every 6 weeks respectively or tripled to about every 3 months or about every 9 weeks respectively. In an alternative embodiment the patient receives gevokizumab at a dose of about 30 mg to about 120 mg about every 2 months or about every 6 weeks in the down-tiration phase or in the maintenance phase independent from any safety issue or throughout the treatment phase. In an alternative embodiment the patient receives gevokizumab at a dose of about 30 mg to about 120 mg about every 3 months or about every 9 weeks in the down-titration phase or in the maintenance phase independent from any safety issue or throughout the treatment phase.

Suitably, the above dose and dosing apply to the use of a functional fragment of gevokizumab according to the present invention.

Gevokizumab or a functional fragment thereof can be administered intravenously or subcutaneously, preferably intravenously.

The dosing regimens disclosed herein are applicable in each and every gevokizumab related embodiment disclosed in this application, including but not limited to monotherapy or in combination with one or more anti-cancer therapeutic agents, used in adjuvant setting or in the first line, 2^{nd} line or 3^{rd} line treatment.

When canakinumab or gevokizumab is used in combination with one or more anti-cancer therapeutic agents, e.g., a chemotherapeutic agent or a checkpoint inhibitor, especially when the one or more therapeutic agents is the SoC of the cancer indication, the dosing interval of canakinumab or gevokizumab can be adjusted to be aligned with the combination partner for the sake of patient convenience. Normally there is no need to change the canakinumab or gevokizumab dose per treatment. For example, canakinumab about 200 mg is administered about every 3 weeks in combination with pembrolizumab. For example canakinumab about 200 mg is administered about every 4 weeks in combination with FOLFOX. For example, canakinumab about 250 mg is administered about every 4 weeks in combination with MBG453.

### Biomarkers

In one aspect, the present invention provides the use of an IL-1β binding antibody or a functional fragment thereof, suitably canakinumab or gevokizumab, in the treatment of MDS, in a patient who has a higher than normal level of C-reactive protein (hsCRP).

As used herein, “C-reactive protein” and “CRP” refers to serum or plasma C-reactive protein, which is typically used as an indicator of the acute phase response to inflammation. Nonetheless, CRP level may become elevated in chronic illnesses such as cancer. The level of CRP in serum or plasma may be given in any concentration, e.g., mg/dl, mg/L, nmol/L. Levels of CRP may be measured by a variety of well-known methods, e.g., radial immunodiffusion, electroimmunoassay, immunoturbidimetry (e.g., particle (e.g., latex)-enhanced turbidimetric immunoassay), ELISA, turbidimetric methods, fluorescence polarization immunoassay, and laser nephelometry. Testing for CRP may employ a standard CRP test or a high sensitivity CRP (hsCRP) test (i.e., a high sensitivity test that is capable of measuring lower levels of CRP in a sample, e.g., using immunoassay or laser nephelometry). Kits for detecting levels of CRP may be purchased from various companies, e.g., Calbiotech, Inc, Cayman Chemical, Roche Diagnostics Corporation, Abazyme, DADE Behring, Abnova Corporation, Aniara Corporation, Bio-Quant Inc., Siemens Healthcare Diagnostics, Abbott Laboratories etc.

As used herein, the term “hsCRP” refers to the level of CRP in the blood (serum or plasma) as measured by high sensitivity CRP testing. For example, Tina-quant C-reactive protein (latex) high sensitivity assay (Roche Diagnostics Corporation) may be used for quantification of the hsCRP level of a subject. Such latex-enhanced turbidimetric immunoassay may be analysed on the Cobas® platform (Roche Diagnostics Corporation) or Roche/Hitachi (e.g., Modular P) analyzer. In the CANTOS trial the hsCRP level was measured by Tina-quant C-reactive protein (latex) high sensitivity assay (Roche Diagnostics Corporation) on the Roche/Hitachi Modular P analyzer, which can be used typically and preferably as the method in measuring hsCRP level. Alternatively the hsCRP level can be measured by another method, for example by another approved companion diagnostic kit, the value of which can be calibrated against the value measured by the Tina-quant method.

Each local laboratory employ a cut-off value for abnormal (high) CRP or hsCRP based on that laboratory's rule for calculating normal maximum CRP, i.e. based on that laboratory's reference standard. A physician generally orders a CRP test from a local laboratory, and the local laboratory determines CRP or hsCRP value and reports normal or abnormal (low or high) CRP using the rule that particular laboratory employs to calculate normal CRP, namely based on its reference standard. Thus whether a patient has a higher than normal level of C-reactive protein (hsCRP) can be determined by the local laboratory where the test is conducted.

It is plausible that an IL-1β antibody or a fragment thereof, such as canakinumab or gevokizumab, is effective in treating MDS, especially when said patient has higher than normal level of hsCRP. Like canakinumab, gevokizumab binds to IL-1β specifically. Unlike canakinumab directly inhibiting the binding of IL-1β to its receptor, gevokizumab is an allosteric inhibitor. It does not inhibit IL-1β from binding to its receptor but prevents the receptor from being activated by IL-1β. Like canakinumab, gevokizumab was tested in a few inflammation based indications and has been shown to effectively reduce inflammation as indicated, for example, by the reduction of hsCRP level in those patients. Furthermore from the available IC50 value, gevokizumab seems to be a more potent IL-1β inhibitor than canakinumab.

Furthermore, the present invention provides effective dosing ranges, within which the hsCRP level can be reduced to a certain threshold, below which more patients with MDS can become responder or below which the same patient can benefit more from the great therapeutic effect of the DRUG of the invention with negligible or tolerable side effects.

In one aspect, the present invention provides high sensitivity C-reactive protein (hsCRP) or CRP for use as a biomarker in the treatment of MDS, with an IL-1β inhibitor, e.g., IL-1β binding antibody or a functional fragment thereof. The level of hsCRP is possibly relevant in determining whether a patient with diagnosed or undiagnosed cancer or is at risk of developing cancer should be treated with an IL-1β binding antibody or a functional fragment thereof. In one embodiment patient is eligible for the treatment and/or prevention if the level of hsCRP is equal to or higher than about 2.5 mg/L, or equal to or higher than about 4.5 mg/L, or equal to or higher than about 7.5 mg/L, or equal to or higher than about 9.5 mg/L, as assessed prior to the administration of the IL-1β binding antibody or a functional fragment thereof.

In one embodiment, the present invention provides the use of an IL-1β binding antibody or a functional fragment thereof, suitably canakinumab or gevokizumab, for the treatment of MDS, in a patient who has high sensitivity C-reactive protein (hsCRP) level equal to or higher than about 2.2 mg/L, equal to or higher than about 4.2 mg/L, equal to or higher than about 6.2 mg/L equal to or higher than about 10.2 mg/L, preferably before first administration of said IL-1β binding antibody or functional fragment thereof. Preferably said patient has a hsCRP level equal to or higher than about 4.2 mg/L. Preferably said patient has a hsCRP level equal to or higher than about 6.2 mg/L. Preferably said patient has a hsCRP level equal to or higher than about 10 mg/L. Preferably said patient has a hsCRP level equal to or higher than about 20 mg/L.

In one aspect the present invention provides an IL-1β binding antibody or a functional fragment thereof for use in the treatment of MDS in a patient, wherein the efficacy of the treatment correlates with the reduction of hsCRP in said patient, comparing to prior treatment. In one embodiment the present invention provides an IL-1β binding antibody or a functional fragment thereof for use in the treatment of MDS, wherein hsCRP level, of said patient has reduced to below about 5.2 mg/L, preferably to below about 3.2 mg/L, preferably to below about 2.2 mg/L, about 6 months, or preferably about 3 months from the first administration of said IL-1β binding antibody or a functional fragment thereof at a proper dose, preferably according to the dosing regimen of the present invention.

In one aspect the present invention provides an IL-1β binding antibody or a functional fragment thereof (e.g., canakinumab or gevokizumab) for use in the treatment of MDS in a patient, wherein the hsCRP level of said patient has reduced by at least about 20%, by about 20-34%, 35% or at least about 50% or at least about 60% about 6 months, or preferably about 3 month from the first administration of said IL-1β binding antibody or a functional fragment thereof at a proper dose, preferably according to the dosing regimen of the present invention, as compared to the hsCRP level just prior to the first administration of the IL-1β binding antibody or a functional fragment thereof, canakinumab or gevokizumab). Further preferably the hsCRP level of said patient has reduced by least about 35% or at least about 50% or at least about 60% after the first administration of the DRUG of the invention according to the dose regimen of the present invention.

In one aspect, the present invention provides IL-6 use as a biomarker in the treatment of MDS, with an IL-1β inhibitor, e.g., IL-1β binding antibody or a functional fragment thereof. The level of IL-6 is possibly relevant in determining whether a patient with diagnosed or undiagnosed cancer or is at risk of developing cancer should be treated with an IL-1β binding antibody or a functional fragment thereof. In one embodiment patient is eligible for the treatment and/or prevention if the level of IL-6 is equal to or higher than about 1.9 pg/ml, higher than about 2 pg/ml, higher than about 2.2 pg/ml, higher than 2.5 pg/ml, higher than about 2.7 pg/ml, higher than about 3 pg/ml, higher than about 3.5 pg/ml, as assessed prior to the administration of the IL-1β binding antibody or a functional fragment thereof. Preferably the patient has an IL-6 level equal to or higher than about 2.5 mg/L

In one aspect the present invention provides an IL-1β binding antibody or a functional fragment thereof for use in the treatment of MDS in a patient, wherein the efficacy of the treatment correlates with the reduction of IL-6 in said patient, comparing to prior treatment. In one embodiment the present invention provides an IL-1β binding antibody or a functional fragment thereof for use in the treatment of cancer, e.g., cancer having at least a partial inflammatory basis, wherein IL-6 level, of said patient has reduced to below about 2.2 pg/ml, preferably to below about 2 pg/ml, preferably to below about 1.9 pg/ml about 6 months, or preferably about 3 months from the first administration of said IL-1β binding antibody or a functional fragment thereof at a proper dose, preferably according to the dosing regimen of the present invention.

In one aspect the present invention provides an IL-1β binding antibody or a functional fragment thereof (e.g., canakinumab or gevokizumab) for use in the treatment of MDS in a patient, wherein the IL-6 level of said patient has reduced by at least about 20%, about 20-34%, about 35% or at least about 50% or at least about 60% about 6 months, or preferably about 3 months from the first administration of said IL-1β binding antibody or a functional fragment thereof (e.g., canakinumab or gevokizumab) at a proper dose, preferably according to the dosing regimen of the present invention, as compared to the IL-6 level just prior to the first administration. Further preferably the IL-6 level of said patient has reduced by least about 35% or at least about 50% or at least about 60% after the first administration of the DRUG of the invention according to the dose regimen of the present invention.

The reduction of the level of hsCRP and the reduction of the level of IL-6 can be used separately or in combination to indicate the efficacy of the treatment or as prognostic markers.

### Inhibition of Angiogenesis

In one aspect, the present invention provides an IL-1β binding antibody or a functional fragment thereof, suitably canakinumab or gevokizumab, for use in a patient in need thereof in the treatment of MDS, wherein a therapeutic amount is administered to inhibit angiogenesis in said patient. Without wishing to be bound by theory, it is hypothesized that the inhibition of the IL-1β pathway can lead to inhibition or reduction of angiogenesis, which is a key event for tumor growth and for tumor metastasis. In clinical settings the inhibition or reduction of angiogenesis can be measured by tumor shrinkage, no tumor growth (stable disease), prevention of metastasis or delay of metastasis.

All the disclosed uses throughout this application, including but not limited to, doses and dosing regimens, combinations, route of administration and biomarkers can be applied to the aspect of inhibition or reduction of angiogenesis. In one embodiment canakinumab or gevokizumab used in combination of one or more anti-cancer therapeutic agents. In one embodiment the one or more chemotherapeutic agents is an anti-Wnt inhibitor, preferably Vantictumab. In one embodiment the one or more therapeutic agents is a VEGF inhibitor, preferably bevacizumab or Ramucirumab.

### Inhibition of Metastasis

Without wishing to be being bound by theory, it is hypothesized that the inhibition of IL-1β pathway can lead to inhibition or reduction of tumor metastasis. Until now there have been no reports on the effects of canakinumab on metastasis. Data presented in example 1 demonstrate that IL-1β activates different pro-metastatic mechanisms at the primary site compared with the metastatic site: Endogenous production of IL-1β by breast cancer cells promotes epithelial to mesenchymal transition (EMT), invasion, migration and organ specific homing. Once tumor cells arrive in the bone environment contact between tumor cells and osteoblasts or bone marrow cells increases IL-1β secretion from all three cell types. These high concentrations of IL-1β cause proliferation of the bone metastatic niche by stimulating growth of disseminated tumor cells into overt metastases. These pro-metastatic processes are inhibited by administration of anti-IL-1β treatments, such as canakinumab or gevokizumab.

Therefore, targeting IL-1β with an IL-1β binding antibody represents a novel therapeutic approach for cancer patients at risk of progressing to metastasis by preventing seeding of new metastases from established tumors and retaining tumor cells already disseminated in the bone in a state of dormancy. The models described have been designed to investigate bone metastasis and although the data show a strong link between IL-1β expression and bone homing, it does not exclude IL-1β involvement in metastasis to other sites.

Accordingly, in one aspect, the present invention provides an IL-1β binding antibody or a functional fragment thereof, suitably canakinumab or gevokizumab, for use in a patient in the treatment of MDS, wherein a therapeutic amount is administered to inhibit metastasis in said patient.

All the disclosed uses throughout this application, including but not limited to, doses and dosing regimens, combinations, route of administration and biomarkers can be applied to the embodiment of metastasis inhibition.

### Prevention

In one aspect the present invention provides the use of an IL-1β binding antibody or a functional fragment thereof, suitably canakinumab or gevokizumab, in the prevention of cancer, e.g., cancers that have at least a partial inflammatory basis in a patient. The term “prevent”, “preventing” or “prevention” as used herein means the prevention or delay of the occurrence of cancer in a subject who is otherwise at high risk of developing cancer. The term “prevent”, “preventing” or “prevention” as used herein also means the prevention or delay of the occurrence of secondary acute myeloid leukemia (AML) in a subject who had an antecedent MDS. MDS frequently progresses to secondary AML.

The term “prevent”, “preventing” or “prevention” as used herein also means the prevention or delay of the occurrence of treatment-related MDS in a subject who has an antecedent, different cancer. MDS is an uncommon but well-recognized complication of chemotherapy for an earlier, different cancer. This is also called treatment-related MDS. The incidence of treatment-related MDS has been related to the use of intensive treatment regimens, often combining high dose chemotherapy and radiotherapy, and the use of adjuvant chemoirradiation in e.g. head and neck, lung, breast and colon cancers and melanoma. Environmental pollution, industrial chemicals and carcinogens may also be predisposing factors, together with the type of primary cancer, intensity of chemotherapy schedule and host characteristics.

The term “prevent”, “preventing” or “prevention” as used herein also means the prevention or delay of the occurrence of MDS after antecedent clonal hematopoiesis of indeterminate potential (CHIP), clonal cytopenia of undetermined significance (CCUS), or idiopathic Cytopenia of Undetermined Significance (ICUS). Clonal Hematopoiesis of Indeterminate Potential (CHIP) is characterized by: presence of at least one somatic mutation that is clinically relevant and is otherwise found in MDS (or other myeloid neoplasms); absence of persistent cytopenia; and/or exclusion of MDS and of all other hematopoietic neoplasms (and other diseases) as the causal underlying condition. Idiopathic Cytopenia of Undetermined Significance (ICUS) is characterized by: relevant cytopenia in one or more lineages persistent for at least about 6 months; not explained by any other disease; and/or diagnostic criteria of myeloid neoplasm not fulfilled. Clonal Cytopenia of Undetermined Significance (CCUS) is characterized by: one or more somatic mutations otherwise found in patients with myeloid neoplasms detected in bone marrow or peripheral blood cells with an allele burden of ≥about 2%; persistent cytopenia (≥about 4 months) in one or more peripheral blood cell lineages; diagnostic criteria of myeloid neoplasm not fulfilled; and/or all other causes of cytopenia and molecular aberration excluded.

In the context of unexplained cytopenia, there is diagnostic value of a somatic mutation analysis (e.g. by NGS) on DNA from peripheral blood cells for patients, which can lead to identification of CHIP or CCUS. Clonal hematopoiesis (CH) is a population of related myeloid cells with an acquired somatic mutation. CH is a characteristic of MDS and leukemias but it is also found in individuals who have no detectable hematologic malignancy. In order to exclude any infiltrative neoplasms CHIP and CCUS also require thorough bone marrow analyses. Should one or more somatic mutations be detected without persistent cytopenia, it is referred to as CHIP, should persistent (≥about 4 months) cytopenia be present such cases are referred to as CCUS. Individuals with CHIP have an approximately 10-fold increased risk of developing a hematologic malignancy, with the risk increasing with the size of the clone and overall risk estimated at about 0.5% to about 1% per year. Transformation to overt malignancy from CHIP or CCUS generally requires the sequential acquisition of multiple mutations.

Acquired somatic mutations and genetic aberrations may result in propagation of MDS clones by selectively activating proinflammatory cytokine responses in the bone marrow (De Mooij Charlotte et al. Blood 2017; 129: 3155-3164 and Carey Alyssa et al., Cell Rep 2017; 18: 3204-3218). Thus, targeting critical innate immune pathways at an early stage may prevent or delay disease progression.

An IL-1β rich environment may augment the selective pressure in the stem cell niche and support the selection and expansion of leukemic over non-leukemic stem cells (De Mooij Charlotte et al. Blood 2017; 129: 3155-3164 and Carey Alyssa et al., Cell Rep 2017; 18: 3204-3218). Thus, therapeutic targeting of overactive IL-1β signaling may enhance normal hematopoiesis while inhibiting pre-/leukemic clones.

Currently, individuals with CHIP are not treated causally because there is no available evidence for any treatment for prevention of the onset of MDS. Therefore, individuals with CHIP are just observed if they develop MDS.

The CANTOS trial showed a high benefit of administrating IL-1β binding antibodies to patients with CHIP and showed that the frequency of unexplained anemia was reduced in patients with CHIP. Therefore, one embodiment of the present invention is preventing individuals with precursor states from progressing to MDS by administering a therapeutically effective amount of an IL-1β binding antibody or a functional fragment thereof, e.g. canakinumab or gevokizumab.

Without wishing to be bound by the theory, it is hypothesized that chronic inflammation, either local or systematic, especially local inflammation, creates an immunosuppressive microenvironment that promotes tumor growth and dissemination. IL-1β binding antibody or a functional fragment thereof reduces chronic inflammation, especially IL-1β mediated chronic inflammation, and thereby prevents or delays the occurrence of cancer in a subject who has otherwise local or systematic chronic inflammation.

One way of determining local or systematic chronic inflammation is through measuring the level of C-reactive protein (hsCRP). In one embodiment, the present invention provides an IL-1β binding antibody or a functional fragment thereof, suitably canakinumab or gevokizumab, for use in the prevention of cancer, e.g., cancers that have at least a partial inflammatory basis, in a subject with a high sensitive C-reactive protein (hsCRP) equal to or higher than about 2 mg/L, equal to or higher than about 3 mg/L, equal to or higher than about 4.2, equal or higher than about 6.5 mg/L, equal to or higher than about 8.5 mg/L, or higher than about 11 mg/L as assessed prior to the administration of the IL-1β binding antibody or functional fragment thereof.

In the prevention setting, it is possible that IL-1β binding antibody or a functional fragment thereof is administered as monotherapy.

In the prevention setting, it is possible that the dose of IL-1β binding antibody or a functional fragment thereof per treatment is not the same as, but likely less than, that in the treatment setting. The prevention dose is likely at most about half, preferably about half of the treatment dose. The interval between the prevention doses is likely not the same as, but likely longer than, that between the treatment doses. It is likely that the interval is doubled or tripled. It is likely that the dose per treatment is the same as in the treatment settings but the dosing interval is elongated. This is preferred as longer dosing intervals provide convenience and hence higher compliance. It is likely that both the dose per treatment is reduced and the dosing interval is elongated.

In one preferred embodiment, canakinumab is administered at a dose of about 100 mg to about 400 mg, preferably about 200 mg monthly, about every other month or about quarterly, preferably subcutaneously or preferably about 100 mg about monthly, about every other month or about quarterly, preferably subcutaneously. In another embodiment, said IL-1β binding antibody is gevokizumab or a functional fragment thereof. In one preferred embodiment, gevokizumab is administered at a dose of about 15 mg to about 60 mg. In one preferred embodiment, gevokizumab is administered about monthly, about every other month or quarterly. In one preferred embodiment, gevokizumab is administered at a dose of about 15 mg monthly, about every other month or about quarterly. In one preferred embodiment, gevokizumab is administered at a dose of about 30 mg monthly, about every other month or about quarterly. In one embodiments gevokizumab is administered subcutaneously. In one embodiments gevokizumab is administered intravenously. In one embodiment canakinumab or gevokizumab is administered by an auto-injector.

In one embodiment the risk of developing cancer, in patients receiving the prevention treatment according to the present invention is reduced by at least about 30%, preferably at least about 50%, preferably at least about 60%, preferably compared to not receiving Treatment of the Invention in the prevention settings.

### Neo-Adjuvant

The term neo-adjuvant treatment is normally understood as radiotherapy or chemotherapy prior to surgery. The purpose of a neo-adjuvant therapy is normally to reduce the tumor size for easy or more complete resection of the tumor. As surgical tumor resection is not possible in MDS, because it is a liquid tumor, neo-adjuvant treatment is not applicable to MDS in a classical sense. However, a different type of surgery is used to treat MDS, which is hematopoietic cell transplantation. In that sense, neo-adjuvant treatment can be applied in MDS, before a hematopoietic cell transplantation. Particularly, as patients often have to wait for a suitable donor, neo-adjuvant treatment can be used during that waiting time.

Chronic inflammation and IL-1β have been associated with a poor histological response to neo-adjuvant therapy and risk of developing cancer (Delitto et al., BMC cancer. 2015′ 15:783). Without wishing to be bound by the theory, by reducing inflammation, IL-1β binding antibody or a functional fragment thereof helps improving the cancer treatment effect, especially synergizing the chemotherapy effect in causing disease improvement.

In one aspect, the present invention provides an IL-1β binding antibody or a functional fragment thereof, suitably canakinumab or gevokizumab, for use, alone or preferably in combination with radiotherapy, or in combination with one or more therapeutic agents, in the treatment of cancer prior to hematopoietic cell transplantation. In one embodiment the one or more therapeutic agents is the SoC treatment in the neo-adjuvant setting in that cancer indication. In one embodiment the one or more therapeutic agents is a checkpoint inhibitor, preferably selected from group consisting of nivolumab, pembrolizumab, atezolizumab, avelumab, durvalumab and spartalizumab, preferred pembrolizumab or nivolumab. In one embodiment the one or more therapeutic agents is a chemotherapeutic agent. In one embodiment the one or more therapeutic agents is a chemotherapeutic agent, wherein the chemotherapeutic agent is not an agent used in targeted therapy.

### First Line Treatment

In one embodiment, the present invention provides an IL-1β antibody or a functional fragment thereof, suitably canakinumab or gevokizumab, for use as the first line treatment of MDS. The term “first line treatment” means said patient is given the IL-1β antibody or a functional fragment thereof before the patient develops resistance to the initial treatment with one or more other therapeutic agents. Preferably one or more other therapeutic agents is a platinum-based mono or combination therapy, a targeted therapy, such a tyrosine inhibitor therapy, a checkpoint inhibitor therapy or any combination thereof. As first line treatment, the IL-1β antibody or a functional fragment thereof, such as canakinumab or gevokizumab, can be administered to a patient as monotherapy or preferably in combination with one or more therapeutic agents, such as a check point inhibitor, particularly a PD-1 or PD-L1 inhibitor, preferably pembrolizumab, with or without one or more small molecule chemotherapeutic agent. In one embodiment as first line treatment, the IL-1β antibody or a functional fragment thereof, such as canakinumab or gevokizumab, can be administered to a patient in combination with the standard of care therapy for MDS. Preferably canakinumab or gevokizumab is administered as the first line treatment until disease progression.

### Second Line Treatment

In one embodiment, the present invention provides an IL-1β antibody or a functional fragment thereof, suitably canakinumab or gevokizumab, for use as the second or third line treatment of MDS. The term “second or third line treatment” means that the IL-1β antibody or a functional fragment thereof is administered to a patient with cancer progression on or after one or more other therapeutic agents, especially cancer progression on or after FDA-approved first line therapy for that cancer. Preferably one or more other therapeutic agents is a chemotherapeutic agent, such as platinum-based mono or combination therapy agent, a targeted therapy agent, such a tyrosine inhibitor therapy agent, a checkpoint inhibitor or any combination thereof. As the second or third line treatment, the IL-1β antibody or a functional fragment thereof can be administered to the patient as monotherapy or preferably in combination with one or more therapeutic agents, including the continuation of the early treatment with the same one or more therapeutic agents. Preferably canakinumab or gevokizumab is administered as the 2^{nd}/3^{rd} line treatment until disease progression.

### Continuous Treatment

In one aspect the present invention also provides an IL-1β binding antibody or a functional fragment thereof, suitably gevokizumab or canakinumab, for use in the treatment of MDS, wherein the IL-1β binding antibody or a functional fragment thereof is administered to a patient in more than one line of treatment.

Without wishing to be bound by the theory, it is hypothesized that, unlike chemotherapeutic agents or targeted therapy agents, which directly kill or inhibit the cancer cells and thereby select resistant cells, DRUG of the invention works on the tumor-microenvironment and does not seem to lead to drug resistance. Furthermore, unlike chemotherapeutic agents or checkpoint inhibitors, IL-1β binding antibody or a functional fragment thereof, such as gevokizumab or canakinumab, has much less undesired side effects. Patients are unlikely to develop intolerance and therefore can continue to receive DRUG of the invention and continue the benefit of elimination or reduction of IL-1β mediated inflammation in the course of cancer treatment.

In one embodiment DRUG of the invention, suitably canakinumab or gevokizumab, can be used in 2, 3, or all lines of the treatment of cancer in the same patient. Treatment line typically includes but is not limited to neo-adjuvant treatment, adjuvant treatment, first line treatment, 2^{nd} line treatment, 3^{rd} line treatment and further line of treatment. A patient normally changes lines of treatment after disease progression or after developing drug resistance to the current treatment. In one embodiment DRUG of the invention is continued after the patient develops resistance to the current treatment. In one embodiment DRUG of the invention is continued to the next line of treatment. In one embodiment DRUG of the invention is continued after disease progression. In one embodiment DRUG of the invention is continued until death or until palliative care.

In one embodiment the present invention provides DRUG of the invention, suitably canakinumab or gevokizumab, for use in re-treating MDS in a patient, wherein the patient was treated with the same DRUG of the invention in the previous treatment. In one embodiment the previous treatment is the neo-adjuvant treatment. In one embodiment the previous treatment is the adjuvant treatment. In one embodiment the previous treatment is the first line treatment. In one embodiment the previous treatment is the second line treatment.

### Combination

In one aspect, the present invention provides an IL-1β binding antibody or a functional fragment thereof, suitably canakinumab or gevokizumab, for use in a patient in need thereof in the treatment of MDS, in combination with a radiotherapy, or in combination with one or more therapeutic agents, e.g., chemotherapeutic agents or e.g., a checkpoint inhibitor, or in combination with both radiotherapy and one or more therapeutic agents.

Without wishing to be bound by the theory, it is believed that typical cancer development requires two steps. Firstly, gene alteration results in cell growth and proliferation is no longer subject to regulation. Secondly, the abnormal tumor cells evade surveillance of the immune system. Inflammation plays important role in the second step. Therefore, control of inflammation can stop cancer development at the early or earlier stage. Thus it is expected that blocking the IL-1β pathway to reduce inflammation would have a general benefit, particularly improvement of the treatment efficacy on top of the standard of care, which is normally mainly to directly inhibit the growth and proliferation of the malignant cells. In one embodiment the one or more therapeutic agents, e.g., chemotherapeutic agents, is the standard of care agents of said cancer, particularly of cancer having at least partial inflammatory basis.

Checkpoint inhibitors de-suppress the immune system through a mechanism different from IL-1β inhibitors. Thus the addition of IL-1β inhibitors, particularly IL-1β binding antibodies or a functional fragment thereof, to the standard checkpoint inhibitor will further activate the immune response, particularly in the tumor microenvironment.

In one embodiment, the one or more therapeutic agents is nivolumab.

In one embodiment, the one or more therapeutic agents is pembrolizumab.

In one embodiment, the one or more therapeutic agents is nivolumab and ipilimumab.

In one embodiment, the one or more chemotherapeutic agents is cabozantinib, or a pharmaceutically acceptable salt thereof.

In one embodiment the or more therapeutic agents is atezolizumab plus bevacizumab.

In one embodiment, the one or more therapeutic agents is bevacizumab.

In one embodiment, the one or more therapeutic agents is a hypomethylating agent (HMA).

In one embodiment, the one or more therapeutic agents is azacitidine (AzaC).

In one embodiment, the one or more therapeutic agents is decitabine. In one embodiment, the one or more therapeutic agents is lenalidomide.

In one embodiment, the one or more therapeutic agents are agents used for intensive induction chemotherapy that is standard for acute myeloid leukemia, including Cytarabine (ara-C); anthracycline drug such as daunorubicin (daunomycin) or idarubicin; fludarabine (Fludara); cladribine; and/or etoposide.

In one embodiment, the one or more therapeutic agents is midostaurin.

In one embodiment, the one or more therapeutic agents is gemtuzumab ozogamicin.

Therapeutic agents are cytotoxic and/or cytostatic drugs (drugs that kill malignant cells, or inhibit their proliferation, respectively) as well as checkpoint inhibitors. Chemotherapeutic agents can be, for example, small molecule agents, biologics agents (e.g., antibodies, cell and gene therapies, cancer vaccines), hormones or other natural or synthetic peptides or polypeptides. Commonly known chemotherapeutic agents include, but are not limited to, platinum agents (e.g., cisplatin, carboplatin, oxaliplatin, nedaplatin, triplatin, lipoplatin, satraplatin, picoplatin), antimetabolites (e.g., methotrexate, 5-Fluorouracil, gemcitabine, pemetrexed, edatrexate), mitotic inhibitors (e.g., paclitaxel, albumin-bound paclitaxel, docetaxel, taxotere, docecad), alkylating agents (e.g., cyclophosphamide, mechlorethamine hydrochloride, ifosfamide, melphalan, thiotepa), vinca alkaloids (e.g., vinblastine, vincristine, vindesine, vinorelbine), topoisomerase inhibitors (e.g., etoposide, teniposide, topotecan, irinotecan, camptothecin, doxorubicin), antitumor antibiotics (e.g., mitomycin C) and/or hormone-modulating agents (e.g., anastrozole, tamoxifen). Examples of anti-cancer agents used for chemotherapy include Cyclophosphamide (Cytoxan®), Methotrexate, 5-Fluorouracil (5-FU), Doxorubicin (Adriamycin®), Prednisone, Tamoxifen (Nolvadex®), Paclitaxel (Taxol®), Albumin-bound paclitaxel (nab-paclitaxel, Abraxane®), Leucovorin, Thiotepa (Thioplex®), Anastrozole (Arimidex®), Docetaxel (Taxotere®), Vinorelbine (Navelbine®), Gemcitabine (Gemzar®), Ifosfamide (Ifex®), Pemetrexed (Alimta®), Topotecan, Melphalan (L-Pam®), Cisplatin (Cisplatinum®, Platinol®), Carboplatin (Paraplatin®), Oxaliplatin (Eloxatin®), Nedaplatin (Aqupla®), Triplatin, Lipoplatin (Nanoplatin®), Satraplatin, Picoplatin, Carmustine (BCNU; BiCNU®), Methotrexate (Folex®, Mexate®), Edatrexate, Mitomycin C (Mutamycin®), Mitoxantrone (Novantrone®), Vincristine (Oncovin®), Vinblastine (Velban®), Vinorelbine (Navelbine®), Vindesine (Eldisine®), Fenretinide, Topotecan, Irinotecan (Camptosar®), 9-amino-camptothecin [9-AC], Biantrazole, Losoxantrone, Etoposide, and Teniposide.

In one embodiment, the preferred combination partner for the IL-1β binding antibody or a functional fragment thereof (e.g., canakinumab or gevokizumab) is a mitotic inhibitor, preferably docetaxel. In one embodiment, the preferred combination partner for canakinumab is a mitotic inhibitor, preferably docetaxel. In one embodiment, the preferred combination partner for gevokizumab is a mitotic inhibitor, preferably docetaxel.

In one embodiment, the preferred combination partner for the IL-1β binding antibody or a functional fragment thereof (e.g., canakinumab or gevokizumab) is a platinum agent, preferably cisplatin. In one embodiment, the preferred combination partner for canakinumab is a platinum agent, preferably cisplatin. In one embodiment, the preferred combination partner for gevokizumab is a platinum agent, preferably cisplatin. In one embodiment, the one or more chemotherapeutic agents is a platinum-based doublet chemotherapy (PT-DC).

Chemotherapy may comprise the administration of a single anti-cancer agent (drug) or the administration of a combination of anti-cancer agents (drugs), for example, one of the following, commonly administered combinations of: carboplatin and taxol; gemcitabine and cisplatin; gemcitabine and vinorelbine; gemcitabine and paclitaxel; cisplatin and vinorelbine; cisplatin and gemcitabine; cisplatin and paclitaxel (Taxol); cisplatin and docetaxel (Taxotere); cisplatin and etoposide; cisplatin and pemetrexed; carboplatin and vinorelbine; carboplatin and gemcitabine; carboplatin and paclitaxel (Taxol); carboplatin and docetaxel (Taxotere); carboplatin and etoposide; carboplatin and pemetrexed. In one embodiment, the one or more chemotherapeutic agent is a platinum-based doublet chemotherapy (PT-DC).

Another class of chemotherapeutic agents are the inhibitors, especially tyrosine kinase inhibitors, that specifically target growth promoting receptors, especially VEGF-R, EGFR, PFGF-R and ALK, or their downstream members of the signalling transduction pathway, the mutation or overproduction of which results in or contributes to the oncogenesis of the tumor at the site (targeted therapies). Exemplary of targeted therapies drugs approved by the Food and Drug administration (FDA) for the targeted treatment of lung cancer include but are not limited bevacizumab (Avastin®), crizotinib (Xalkori®), erlotinib (Tarceva®), gefitinib (Iressa®), afatinib dimaleate (Gilotrif®), ceritinib (LDK378/Zykadia™), everolimus (Afinitor®), ramucirumab (Cyramza®), osimertinib (Tagrisso™), necitumumab (Portrazza™), alectinib (Alecensa®), atezolizumab (Tecentriq™), brigatinib (Alunbrig™), trametinib (Mekinist®), dabrafenib (Tafinlar®), sunitinib (Sutent®) and cetuximab (Erbitux®).

In one embodiment the one or more therapeutic agent to be combined with the IL-1β binding antibody or fragment thereof, suitably canakinumab or gevokizumab, is a checkpoint inhibitor. In one further embodiment, said check-point inhibitor is nivolumab. In one embodiment said check-point inhibitor is pembrolizumab. In one further embodiment, said check-point inhibitor is atezolizumab. In one further embodiment, said check-point inhibitor is PDR-001 (spartalizumab). In one embodiment, said check-point inhibitor is durvalumab. In one embodiment, said check-point inhibitor is avelumab. Immunotherapies that target immune checkpoints, also known as checkpoint inhibitors, are currently emerging as key agents in cancer therapy. The immune checkpoint inhibitor can be an inhibitor of the receptor or an inhibitor of the ligand. Examples of the inhibiting targets include but not limited to a co-inhibitory molecule (e.g., a PD-1 inhibitor (e.g., an anti-PD-1 antibody molecule), a PD-L1 inhibitor (e.g., an anti-PD-L1 antibody molecule), a PD-L2 inhibitor (e.g., an anti-PD-L2 antibody molecule), a LAG-3 inhibitor (e.g., an anti-LAG-3 antibody molecule), a TIM-3 inhibitor (e.g., an anti-TIM-3 antibody molecule), an activator of a co-stimulatory molecule (e.g., a GITR agonist (e.g., an anti-GITR antibody molecule)), a cytokine (e.g., IL-15 complexed with a soluble form of IL-15 receptor alpha (IL-15Ra)), an inhibitor of cytotoxic T-lymphocyte-associated protein 4 (e.g., an anti-CTLA-4 antibody molecule) or any combination thereof.

In a preferred embodiment, the check-point inhibitor is MBG453 (Novartis).

### PD-1 Inhibitors

In one aspect of the invention, the IL-1β inhibitor or a functional fragment thereof is administered together with a PD-1 inhibitor. In one some embodiment the PD-1 inhibitor is chosen from PDR001 (spartalizumab) (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

In one embodiment, the PD-1 inhibitor is an anti-PD-1 antibody. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

TABLE 1
Amino acid and nucleotide sequences of exemplary anti-PD-1 antibody
molecules

| | | |
|---|---|---|
| BAP049-Clone-B HC | VH | EVQLVQSGAEVKKPGESLRISCKGSGYTFTTYWMHWVRQAT |
| SEQ ID NO: 506 | | GQGLEWMGNIYPGTGGSNFDEKFKNRVTITADKSTSTAYMEL |
| | | SSLRSEDTAVYYCTRWTTGTGAYWGQGTTVTVSS |
| BAP049-Clone-B LC | VL | EIVLTQSPATLSLSPGERATLSCKSSQSLLDSGNQKNFLTWYQQ |
| SEQ ID NO: 516 | | KPGKAPKLLIYWASTRESGVPSRFSGSGSGTDFTFTISSLQPEDI |
| | | ATYYCQNDYSYPYTFGQGTKVEIK |
| BAP049-Clone-E HC | VH | EVQLVQSGAEVKKPGESLRISCKGSGYTFTTYWMHWVRQAT |
| SEQ ID NO: 506 | | GQGLEWMGNIYPGTGGSNFDEKFKNRVTITADKSTSTAYMEL |
| | | SSLRSEDTAVYYCTRWTTGTGAYWGQGTTVTVSS |
| BAP049-Clone-E LC | VL | EIVLTQSPATLSLSPGERATLSCKSSQSLLDSGNQKNFLTWYQQ |
| SEQ ID NO: 520 | | KPGQAPRLLIYWASTRESGVPSRFSGSGSGTDFTFTISSLEAEDA |
| | | ATYYCQNDYSYPYTFGQGTKVEIK |

In one embodiment, the anti-PD-1 antibody is spartalizumab.

In one embodiment, the anti-PD-1 antibody is Nivolumab.

In one embodiment, the anti-PD-1 antibody molecule is Pembrolizumab.

In one embodiment, the anti-PD-1 antibody molecule is Pidilizumab.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. Other exemplary anti-PD-1 molecules include REGN2810 (Regeneron), PF-06801591 (Pfizer), BGB-A317/BGB-108 (Beigene), INCSHR1210 (Incyte) and TSR-042 (Tesaro).

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

### PD-L1 Inhibitors

In one aspect of the invention, the IL-1β inhibitor or a functional fragment thereof is administered together with a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

TABLE 2
Amino acid and nucleotide sequences of exemplary anti-PD-L1 antibody
molecules

| | | |
|---|---|---|
| BAP058-Clone O HC | VH | EVQLVQSGAEVKKPGATVKISCKVSGYTFTSYWMYWVRQA |
| SEQ ID NO: 606 | | RGQRLEWIGRIDPNSGSTKYNEKFKNRFTISRDNSKNTLYLQ |
| | | MNSLRAEDTAVYYCARDYRKGLYAMDYWGQGTTVTVSS |
| BAP058-Clone O LC | VL | AIQLTQSPSSLSASVGDRVTITCKASQDVGTAVAWYLQKPGQ |
| SEQ ID NO: 616 | | SPQLLIYWASTRHTGVPSRFSGSGSGTDFTFTISSLEAEDAATY |
| | | YCQQYNSYPLTFGQGTKVEIK |
| BAP058-Clone N HC | VH | EVQLVQSGAEVKKPGATVKISCKVSGYTFTSYWMYWVRQA |
| SEQ ID NO: 620 | | TGQGLEWMGRIDPNSGSTKYNEKFKNRVTITADKSTSTAYME |
| | | LSSLRSEDTAVYYCARDYRKGLYAMDYWGQGTTVTVSS |
| BAP058-Clone N LC | VL | DVVMTQSPLSLPVTLGQPASISCKASQDVGTAVAWYQQKPG |
| SEQ ID NO: 624 | | QAPRLLIYWASTRHTGVPSRFSGSGSGTEFTLTISSLQPDDFAT |
| | | YYCQQYNSYPLTFGQGTKVEIK |

In one embodiment, the anti-PD-L1 antibody molecule is Atezolizumab (Genentech/Roche), also known as MPDL3280A, RG7446, RO5541267, YW243.55.S70, or TECENTRIQ™. Atezolizumab and other anti-PD-L1 antibodies are disclosed in U.S. Pat. No. 8,217,149, incorporated by reference in its entirety.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

### LAG-3 Inhibitors

In one aspect of the invention, the IL-1β inhibitor or a functional fragment thereof is administered together with a LAG-3 inhibitor. In some embodiments, the LAG-3 inhibitor is chosen from LAG525 (Novartis), BMS-986016 (Bristol-Myers Squibb), TSR-033 (Tesaro), IMP731 or GSK2831781 and IMP761 (Prima BioMed).

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

TABLE 3
Amino acid and nucleotide sequences of exemplary anti-LAG-3 antibody
molecules

| | | |
|---|---|---|
| BAP050-Clone I HC | VH | QVQLVQSGAEVKKPGASVKVSCKASGFTLTNYGMNWVRQAR |
| SEQ ID NO:706 | | GQRLEWIGWINTDTGEPTYADDFKGRFVFSLDTSVSTAYLQISS |
| | | LKAEDTAVYYCARNPPYYYGTNNAEAMDYWGQGTTVTVSS |
| BAP050-Clone I LC | VL | DIQMTQSPSSLSASVGDRVTITCSSSQDISNYLNWYLQKPGQSP |
| SEQ ID NO: 718 | | QLLIYYTSTLHLGVPSRFSGSGSGTEFTLTISSLQPDDFATYYCQ |
| | | QYYNLPWTFGQGTKVEIK |
| BAP050-Clone J HC | VH | QVQLVQSGAEVKKPGASVKVSCKASGFTLTNYGMNWVRQAP |
| SEQ ID NO: 724 | | GQGLEWMGWINTDTGEPTYADDFKGRFVFSLDTSVSTAYLQI |
| | | SSLKAEDTAVYYCARNPPYYYGTNNAEAMDYWGQGTTVTVS |
| | | S |
| BAP050-Clone J LC | VL | DIQMTQSPSSLSASVGDRVTITCSSSQDISNYLNWYQQKPGKAP |
| SEQ ID NO: 730 | | KLLIYYTSTLHLGIPPRFSGSGYGTDFTLTINNIESEDAAYYFCQ |
| | | QYYNLPWTFGQGTKVEIK |

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 4.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 4.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 4
Amino acid sequences of exemplary anti-LAG-3 antibody molecules
BMS-986016

| | | |
|---|---|---|
| SEQ ID NO: 762 | Heavy chain | QVQLQQWGAGLLKPSETLSLTCAVYGGSFSDYYWNWIRQPPGKG |
| | | LEWIGEINHRGSTNSNPSLKSRVTLSLDTSKNQFSLKLRSVTAADTA |
| | | VYYCAFGYSDYEYNWFDPWGQGTLVTVSSASTKGPSVFPLAPCSR |
| | | STSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 763 | Light chain | EIVLTQSPATLSLSPGERATLSCRASQSISSYLAWYQQKPGQAPRLL |
| | | IYDASNRATGIPARFSGSGSGTDFTLTISSLEPEDFAVYYCQQRSNW |
| | | PLTFGQGTNLEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |

IMP731

| | | |
|---|---|---|
| SEQ ID NO: 764 | Heavy chain | QVQLKESGPGLVAPSQSLSITCTVSGFSLTAYGVNWVRQPPGKGLE |
| | | WLGMIWDDGSTDYNSALKSRLSISKDNSKSQVFLKMNSLQTDDTA |
| | | RYYCAREGDVAFDYWGQGTTLTVSSASTKGPSVFPLAPSSKSTSG |
| | | GTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKE |
| | | YKCKVSNKALPAPIEKTISKAKGQPREPQVYTLPPSRDELTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPGK |
| SEQ ID NO: 765 | Light chain | DIVMTQSPSSLAVSVGQKVTMSCKSSQSLLNGSNQKNYLAWYQQ |
| | | KPGQSPKLLVYFASTRDSGVPDRFIGSGSGTDFTLTISSVQAEDLAD |
| | | YFCLQHFGTPPTFGGGTKLEIKRTVAAPSVFIFPPSDEQLKSGTASV |
| | | VCLLNNFYPREAKVQWKVDNALQSGNSQESVTEQDSKDSTYSLSS |
| | | TLTLSKADYEKHKVYACEVTHQGLSSPVTKSFNRGEC |

### TIM-3 Inhibitors

Given the immunomodulatory role of TIM-3 in both innate and adaptive immunity, as well as its expression on leukemic stem cells in AML and MDS, a TIM-3 inhibitor may not only help to restore an anti-tumor immune response, but may additionally directly target MDS stem cells. As a result, a TIM-3 inhibitor may have direct and indirect disease-modifying activity in low-risk MDS which could be augmented by IL-1β blockade, which is a therapy directed at a pro-inflammatory pathway.

### Exemplary TIM-3 Inhibitors

In certain embodiments, the combination described herein comprises an anti-TIM3 antibody molecule. In one embodiment, the anti-TIM-3 antibody molecule is disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of ABTIM3-hum 11 or ABTIM3-hum03 disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 5. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 5.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%. or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 5
Amino acid and nucleotide sequences of exemplary anti-TIM-3 antibody
molecules
ABTIM3-hum11

| | | |
|---|---|---|
| SEQ ID NO: 801 (Kabat) | HCDR1 | SYNMH |
| SEQ ID NO: 802 (Kabat) | HCDR2 | DIYPGNGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 805 (Chothia) | HCDR2 | YPGNGD |
| SEQ ID NO: 803 (Chothia) | HCDR3 | VGGAFPMDY |
| SEQ ID NO: 806 | VH | QVQLVQSGAEVKKPGSSVKVSCKASGYTFTSYNMHWVRQAPG |
| | | QGLEWMGDIYPGNGDTSYNQKFKGRVTITADKSTSTVYMELSS |
| | | LRSEDTAVYYCARVGGAFPMDYWGQGTTVTVSS |
| SEQ ID NO: 807 | DNA VH | CAGGTGCAGCTGGTGCAGTCAGGCGCCGAAGTGAAGAAACC |
| | | CGGCTCTAGCGTGAAAGTTTCTTGTAAAGCTAGTGGCTACAC |
| | | CTTCACTAGCTATAATATGCACTGGGTTCGCCAGGCCCCAGG |
| | | GCAAGGCCTCGAGTGGATGGGCGATATCTACCCCGGGAACGG |
| | | CGACACTAGTTATAATCAGAAGTTTAAGGGTAGAGTCACTAT |
| | | CACCGCCGATAAGTCTACTAGCACCGTCTATATGGAACTGAG |
| | | TTCCCTGAGGTCTGAGGACACCGCCGTCTACTACTGCGCTAG |
| | | AGTGGGCGGAGCCTTCCCTATGGACTACTGGGGTCAAGGCAC |
| | | TACCGTGACCGTGTCTAGC |
| SEQ ID NO:808 | Heavy | QVQLVQSGAEVKKPGSSVKVSCKASGYTFTSYNMHWVRQAPG |
| | chain | QGLEWMGDIYPGNGDTSYNQKFKGRVTITADKSTSTVYMELSS |
| | | LRSEDTAVYYCARVGGAFPMDYWGQGTTVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVVV |
| | | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLG |
| SEQ ID NO: 809 | DNA | CAGGTGCAGCTGGTGCAGTCAGGCGCCGAAGTGAAGAAACC |
| | heavy | CGGCTCTAGCGTGAAAGTTTCTTGTAAAGCTAGTGGCTACAC |
| | chain | CTTCACTAGCTATAATATGCACTGGGTTCGCCAGGCCCCAGG |
| | | GCAAGGCCTCGAGTGGATGGGCGATATCTACCCCGGGAACGG |
| | | CGACACTAGTTATAATCAGAAGTTTAAGGGTAGAGTCACTAT |
| | | CACCGCCGATAAGTCTACTAGCACCGTCTATATGGAACTGAG |
| | | TTCCCTGAGGTCTGAGGACACCGCCGTCTACTACTGCGCTAG |
| | | AGTGGGCGGAGCCTTCCCTATGGACTACTGGGGTCAAGGCAC |
| | | TACCGTGACCGTGTCTAGCGCTAGCACTAAGGGCCCGTCCGT |
| | | GTTCCCCCTGGCACCTTGTAGCCGGAGCACTAGCGAATCCAC |
| | | CGCTGCCCTCGGCTGCCTGGTCAAGGATTACTTCCCGGAGCC |
| | | CGTGACCGTGTCCTGGAACAGCGGAGCCCTGACCTCCGGAGT |
| | | GCACACCTTCCCCGCTGTGCTGCAGAGCTCCGGGCTGTACTC |
| | | GCTGTCGTCGGTGGTCACGGTGCCTTCATCTAGCCTGGGTACC |
| | | AAGACCTACACTTGCAACGTGGACCACAAGCCTTCCAACACT |
| | | AAGGTGGACAAGCGCGTCGAATCGAAGTACGGCCCACCGTG |
| | | CCCGCCTTGTCCCGCGCCGGAGTTCCTCGGCGGTCCCTCGGTC |
| | | TTTCTGTTCCCACCGAAGCCCAAGGACACTTTGATGATTTCCC |
| | | GCACCCCTGAAGTGACATGCGTGGTCGTGGACGTGTCACAGG |
| | | AAGATCCGGAGGTGCAGTTCAATTGGTACGTGGATGGCGTCG |
| | | AGGTGCACAACGCCAAAACCAAGCCGAGGGAGGAGCAGTTC |
| | | AACTCCACTTACCGCGTCGTGTCCGTGCTGACGGTGCTGCATC |
| | | AGGACTGGCTGAACGGGAAGGAGTACAAGTGCAAAGTGTCC |
| | | AACAAGGGACTTCCTAGCTCAATCGAAAAGACCATCTCGAAA |
| | | GCCAAGGGACAGCCCCGGGAACCCCAAGTGTATACCCTGCCA |
| | | CCGAGCCAGGAAGAAATGACTAAGAACCAAGTCTCATTGACT |
| | | TGCCTTGTGAAGGGCTTCTACCCATCGGATATCGCCGTGGAA |
| | | TGGGAGTCCAACGGCCAGCCGGAAAACAACTACAAGACCAC |
| | | CCCTCCGGTGCTGGACTCAGACGGATCCTTCTTCCTCTACTCG |
| | | CGGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 816 | VL | AIQLTQSPSSLSASVGDRVTITCRASESVEYYGTSLMQWYQQKP |
| | | GKAPKLLIYAASNVESGVPSRFSGSGSGTDFTLTISSLQPEDFATY |
| | | FCQQSRKDPSTFGGGTKVEIK |
| SEQ ID NO: 817 | DNA VL | GCTATTCAGCTGACTCAGTCACCTAGTAGCCTGAGCGCTAGT |
| | | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAG |
| SEQ ID NO: 818 | Light | AIQLTQSPSSLSASVGDRVTITCRASESVEYYGTSLMQWYQQKP |
| | chain | GKAPKLLIYAASNVESGVPSRFSGSGSGTDFTLTISSLQPEDFATY |
| | | FCQQSRKDPSTFGGGTKVEIKRTVAAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC |
| SEQ ID NO: 819 | DNA light | GCTATTCAGCTGACTCAGTCACCTAGTAGCCTGAGCGCTAGT |
| | chain | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCAC |
| | | CGAGCAGGACAGCAAGGACTCCACCTACAGCCTGAGCAGCA |
| | | CCCTGACCCTGAGCAAGGCCGACTACGAGAAGCATAAGGTGT |
| | | ACGCCTGCGAGGTGACCCACCAGGGCCTGTCCAGCCCCGTGA |
| | | CCAAGAGCTTCAACAGGGGCGAGTGC |

ABTIM3-hum03

| | | |
|---|---|---|
| SEQ ID NO: 801 (Kabat) | HCDR1 | SYNMH |
| SEQ ID NO: 820 (Kabat) | HCDR2 | DIYPGQGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 821 (Chothia) | HCDR2 | YPGQGD |
| SEQ ID NO: 803 (Chothia) | HCDR3 | VGGAFPMDY |
| SEQ ID NO: 822 | VH | QVQLVQSGAEVKKPGASVKVSCKASGYTFTSYNMHWVRQAPG |
| | | QGLEWIGDIYPGQGDTSYNQKFKGRATMTADKSTSTVYMELSS |
| | | LRSEDTAVYYCARVGGAFPMDYWGQGTLVTVSS |
| SEQ ID NO: 823 | DNA VH | CAGGTGCAGCTGGTGCAGTCAGGCGCCGAAGTGAAGAAACC |
| | | CGGCGCTAGTGTGAAAGTTAGCTGTAAAGCTAGTGGCTATAC |
| | | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGTT |
| | | CCCTGAGGTCTGAGGACACCGCCGTCTACTACTGCGCTAGAG |
| | | TGGGCGGAGCCTTCCCAATGGACTACTGGGGTCAAGGCACCC |
| | | TGGTCACCGTGTCTAGC |
| SEQ ID NO: 824 | Heavy | QVQLVQSGAEVKKPGASVKVSCKASGYTFTSYNMHWVRQAPG |
| | chain | QGLEWIGDIYPGQGDTSYNQKFKGRATMTADKSTSTVYMELSS |
| | | LRSEDTAVYYCARVGGAFPMDYWGQGTLVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVVV |
| | | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLG |
| SEQ ID NO: 825 | DNA | CAGGTGCAGCTGGTGCAGTCAGGCGCCGAAGTGAAGAAACC |
| | heavy | CGGCGCTAGTGTGAAAGTTAGCTGTAAAGCTAGTGGCTATAC |
| | chain | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGTT |
| | | CCCTGAGGTCTGAGGACACCGCCGTCTACTACTGCGCTAGAG |
| | | TGGGCGGAGCCTTCCCAATGGACTACTGGGGTCAAGGCACCC |
| | | TGGTCACCGTGTCTAGCGCTAGCACTAAGGGCCCGTCCGTGT |
| | | TCCCCCTGGCACCTTGTAGCCGGAGCACTAGCGAATCCACCG |
| | | CTGCCCTCGGCTGCCTGGTCAAGGATTACTTCCCGGAGCCCGT |
| | | GACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAG |
| | | ACCTACACTTGCAACGTGGACCACAAGCCTTCCAACACTAAG |
| | | GTGGACAAGCGCGTCGAATCGAAGTACGGCCCACCGTGCCCG |
| | | CCTTGTCCCGCGCCGGAGTTCCTCGGCGGTCCCTCGGTCTTTC |
| | | TGTTCCCACCGAAGCCCAAGGACACTTTGATGATTTCCCGCA |
| | | CCCCTGAAGTGACATGCGTGGTCGTGGACGTGTCACAGGAAG |
| | | ATCCGGAGGTGCAGTTCAATTGGTACGTGGATGGCGTCGAGG |
| | | TGCACAACGCCAAAACCAAGCCGAGGGAGGAGCAGTTCAAC |
| | | TCCACTTACCGCGTCGTGTCCGTGCTGACGGTGCTGCATCAGG |
| | | ACTGGCTGAACGGGAAGGAGTACAAGTGCAAAGTGTCCAAC |
| | | AAGGGACTTCCTAGCTCAATCGAAAAGACCATCTCGAAAGCC |
| | | AAGGGACAGCCCCGGGAACCCCAAGTGTATACCCTGCCACCG |
| | | AGCCAGGAAGAAATGACTAAGAACCAAGTCTCATTGACTTGC |
| | | CTTGTGAAGGGCTTCTACCCATCGGATATCGCCGTGGAATGG |
| | | GAGTCCAACGGCCAGCCGGAAAACAACTACAAGACCACCCC |
| | | TCCGGTGCTGGACTCAGACGGATCCTTCTTCCTCTACTCGCGG |
| | | CTGACCGTGGATAAGAGCAGATGGCAGGAGGGAAATGTGTT |
| | | CAGCTGTTCTGTGATGCATGAAGCCCTGCACAACCACTACAC |
| | | TCAGAAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 826 | VL | DIVLTQSPDSLAVSLGERATINCRASESVEYYGTSLMQWYQQKP |
| | | GQPPKLLIYAASNVESGVPDRFSGSGSGTDFTLTISSLQAEDVAV |
| | | YYCQQSRKDPSTFGGGTKVEIK |
| SEQ ID NO: 827 | DNA VL | GATATCGTCCTGACTCAGTCACCCGATAGCCTGGCCGTCAGC |
| | | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAG |
| SEQ ID NO: 828 | Light | DIVLTQSPDSLAVSLGERATINCRASESVEYYGTSLMQWYQQKP |
| | chain | GQPPKLLIYAASNVESGVPDRFSGSGSGTDFTLTISSLQAEDVAV |
| | | YYCQQSRKDPSTFGGGTKVEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 829 | DNA light | GATATCGTCCTGACTCAGTCACCCGATAGCCTGGCCGTCAGC |
| | chain | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCAC |
| | | CGAGCAGGACAGCAAGGACTCCACCTACAGCCTGAGCAGCA |
| | | CCCTGACCCTGAGCAAGGCCGACTACGAGAAGCATAAGGTGT |
| | | ACGCCTGCGAGGTGACCCACCAGGGCCTGTCCAGCCCCGTGA |
| | | CCAAGAGCTTCAACAGGGGCGAGTGC |

In one embodiment, the anti-TIM-3 antibody molecule includes at least one or two heavy chain variable domain (optionally including a constant region), at least one or two light chain variable domain (optionally including a constant region), or both, comprising the amino acid sequence of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences. The anti-TIM-3 antibody molecule, optionally, comprises a leader sequence from a heavy chain, a light chain, or both, as shown in US 2015/0218274; or a sequence substantially identical thereto.

In yet another embodiment, the anti-TIM-3 antibody molecule includes at least one, two, or three complementarity determining regions (CDRs) from a heavy chain variable region and/or a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In yet another embodiment, the anti-TIM-3 antibody molecule includes at least one, two, or three CDRs (or collectively all of the CDRs) from a heavy chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Table 1-4.

In yet another embodiment, the anti-TIM-3 antibody molecule includes at least one, two, or three CDRs (or collectively all of the CDRs) from a light chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4. In certain embodiments, the anti-TIM-3 antibody molecule includes a substitution in a light chain CDR, e.g., one or more substitutions in a CDR1, CDR2 and/or CDR3 of the light chain.

In another embodiment, the anti-TIM-3 antibody molecule includes at least one, two, three, four, five or six CDRs (or collectively all of the CDRs) from a heavy and light chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4.

In another embodiment, the anti-TIM3 antibody molecule is MBG453. Without wising to be bound by theory, it is typically believed that MBG453 is a high-affinity, ligand-blocking, humanized anti-TIM-3 IgG4 antibody which can block the binding of TIM-3 to phosphatidyserine (PtdSer). Historically MBG453 is often misspelt as MGB453.

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 6. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of F38-2E2.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of LY3321367.

In one embodiment, the anti-TIM-3 antibody molecule is Sym023 (Symphogen). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of Sym023.

In one embodiment, the anti-TIM-3 antibody molecule is BGB-A425 (Beigene). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BGB-A425.

In one embodiment, the anti-TIM-3 antibody molecule is INCAGN-2390 (Agenus/Incyte). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of INCAGN-2390.

In one embodiment, the anti-TIM-3 antibody molecule is MBS-986258 (BMS/Five Prime). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of MBS-986258.

In one embodiment, the anti-TIM-3 antibody molecule is RO-7121661 (Roche). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of RO-7121661.

In one embodiment, the anti-TIM-3 antibody molecule is LY-3415244 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of LY-3415244.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 6
Amino acid sequences of other exemplary anti-TIM-3 antibody molecules
APE5137

| | | |
|---|---|---|
| SEQ ID NO: 830 | VH | EVQLLESGGGLVQPGGSLRLSCAAASGFTFSSYDMSWVRQAPGKGLDWVS |
| | | TISGGGTYTYYQDSVKGRFTISRDNSKNTLYLQMNSLRAEDTAVYYCASMD |
| | | YWGQGTTVTVSSA |
| SEQ ID NO: 831 | VL | DIQMTQSPSSLSASVGDRVTITCRASQSIRRYLNWYHQKPGKAPKLLIYGAS |
| | | TLQSGVPSRFSGSGSGTDFTLTISSLQPEDFAVYYCQQSHSAPLTFGGGTKVE |
| | | IKR |

APE5121

| | | |
|---|---|---|
| SEQ ID NO: 832 | VH | EVQVLESGGGLVQPGGSLRLYCVASGFTFSGSYAMSWVRQAPGKGLEWVS |
| | | AISGSGGSTYYADSVKGRFTISRDNSKNTLYLQMNSLRAEDTAVYYCAKKY |
| | | YVGPADYWGQGTLVTVSSG |
| SEQ ID NO: 833 | VL | DIVMTQSPDSLAVSLGERATINCKSSQSVLYSSNNKNYLAWYQHKPGQPPK |
| | | LLIYWASTRESGVPDRFSGSGSGTDFTLTISSLQAEDVAVYYCQQYYSSPLTF |
| | | GGGTKIEVK |

In one aspect of the invention, the IL-1β binding antibody or functional fragment thereof, suitably canakinumab or gevokizumab, for use in the treatment of MDS in a patient in need thereof, is administered in combination with a TIM-3 inhibitor. In some embodiments, the TIM-3 inhibitor is MBG453 (Novartis) or TSR-022 (Tesaro). In a preferred embodiment, the TIM-3 inhibitor is MBG453 (Novartis).

If MBG453 is administered in combination with canakinumab every 4 weeks, then a suitable dose for MBG453 is about 800 mg about every 4 weeks, and a suitable dose for canakinumab is about 250 mg about every 4 weeks. Based on population PK analysis, a 250 mg Q4W dosing schedule of canakinumab would result in comparable PK to the 200 mg Q3W regimen, which is being tested in other oncology indications. If MBG453 is administered in combination with canakinumab every 3 weeks, then a suitable dose for MBG453 is about 600 mg about every 3 weeks, and a suitable dose for canakinumab is about 200 mg about every 3 weeks. Thus, doses of about 800 mg MBG453 about every 4 weeks (Q4W), about 600 mg MBG453 about every 3 weeks (Q3W) and about 400 mg MBG453 about every 2 weeks (Q2W) are also suitable when MBG453 is administered in combination with canakinumab.

In one embodiment, the present invention provides an IL-1β binding antibody or a functional fragment thereof, suitably canakinumab or gevokizumab, for use in the treatment of anemia in MDS in a patient in need thereof, suitably anemia in low risk MDS, in combination with MBG453.

In the CANTOS trial anemia was reduced

In one embodiment, the IL-1β binding antibody or functional fragment thereof, suitably canakinumab or gevokizumab, for use in the treatment of MDS in a patient in need thereof, is administered in combination with MBG453 is administered to patients with lower risk MDS with anemia, thrombocytopenia or neutropenia that are considered to require treatment by the treating physician and for which there are no standard of care treatment options.

In one embodiment about 250 mg canakinumab about Q4W in combination with 800 mg MBG453 about Q4W is administered to patients with a confirmed diagnosis of IPSS-R-defined very low, low or intermediate-risk myelodysplastic syndrome (MDS) with one or more of the following:
Anemia that is relapsed, refractory or intolerant to ESAs, and considered to require treatment by the treating physician
Anemia that is ESA-naive with EPO level about ≥500 mU/mL and considered to require treatment by the treating physician
Thrombocytopenia amenable to response assessments by IWG and considered to require treatment by the treating physician
Neutropenia amenable to response assessments by IWG and which is relapsed, refractory or intolerant to growth factors and considered to require treatment by the treating physician

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

The antibody molecules described herein can be made by vectors, host cells, and methods described in US 2015/0218274, incorporated by reference in its entirety.

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 6. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

### GITR Agonists

In one aspect of the invention, the IL-1β inhibitor or a functional fragment thereof is administered together with a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises a VH comprising the amino acid sequence of SEQ ID NO: 901 and a VL comprising the amino acid sequence of SEQ ID NO: 902.

TABLE 7
Amino acid and nucleotide sequences of exemplary anti-GITR antibody
molecule

| | | |
|---|---|---|
| MAB7 | | |
| SEQ ID NO: 901 | VH | EVQLVESGGGLVQSGGSLRLSCAASGFSLSSYGVDWVRQA |
| | | PGKGLEWVGVIWGGGGTYYASSLMGRFTISRDNSKNTLYL |
| | | QMNSLRAEDTAVYYCARHAYGHDGGFAMDYWGQGTLVT |
| | | VSS |
| SEQ ID NO: 902 | VL | EIVMTQSPATLSVSPGERATLSCRASESVSSNVAWYQQRPG |
| | | QAPRLLIYGASNRATGIPARFSGSGSGTDFTLTISRLEPEDFA |
| | | VYYCGQSYSYPFTFGQGTKLEIK |

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 8.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety.

In one embodiment, the anti-GITR antibody molecule is AMG 228 (Amgen). AMG 228 and other anti-GITR anti

bodies are disclosed, e.g., in U.S. Pat. No. 9,464,139 and WO 2015/031667, incorporated by reference in their entirety.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76 (14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 8
Amino acid sequence of exemplary anti-GITR antibody molecules

| | | |
|---|---|---|
| BMS-986156 | | |
| SEQ ID NO: 920 | VH | QVQLVESGGGVVQPGRSLRLSCAASGFTFSSYGMHWVRQAPGK |
| | | GLEWVAVIWYEGSNKYYADSVKGRFTISRDNSKNTLYLQMNSL |
| | | RAEDTAVYYCARGGSMVRGDYYYGMDVWGQGTTVTVSS |
| SEQ ID NO: 921 | VL | AIQLTQSPSSLSASVGDRVTITCRASQGISSALAWYQQKPGKAPK |
| | | LLIYDASSLESGVPSRFSGSGSGTDFTLTISSLQPEDFATYYCQQF |
| | | NSYPYTFGQGTKLEIK |

IL15/IL-15Ra Complexes

In one aspect of the invention, the IL-1β inhibitor or a functional fragment thereof is administered together with an IL-15/IL-15Ra complex. In some embodiments, the IL-15/IL-15Ra complex is chosen from NIZ985 (Novartis), ATL-803 (Altor) or CYP0150 (Cytune).

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table 9 and the soluble form of human IL-15Ra comprises an amino acid sequence of SEQ ID NO:1002 in Table 9, as described in WO 2014/066527, incorporated by reference in its entirety. The molecules described herein can be made by vectors, host cells, and methods described in WO 2007/084342, incorporated by reference in its entirety.

TABLE 9
Amino acid and nucleotide sequences of exemplary IL-15/IL-15Ra complexes

| | | |
|---|---|---|
| NIZ985 | | |
| SEQ ID NO: | Human IL-15 | NWVNVISDLKKIEDLIQSMHIDATLYTESDVHPSCKVTAMKCFLLE |
| 1001 | | LQVISLESGDASIHDTVENLIILANNSLSSNGNVTESGCKECEELEEK |
| | | NIKEFLQSFVHIVQMFINTS |
| SEQ ID NO: | Human Soluble | ITCPPPMSVEHADIWVKSYSLYSRERYICNSGFKRKAGTSSLTECVL |
| 1002 | IL-15Ra | NKATNVAHWTTPSLKCIRDPALVHQRPAPPSTVTTAGVTPQPESLS |
| | | PSGKEPAASSPSSNNTAATTAAIVPGSQLMPSKSPSTGTTEISSHESS |
| | | HGTPSQTTAKNWELTASASHQPPGVYPQG |

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 10.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 10.

TABLE 10
Amino acid sequences of other exemplary IL-15/IL-15Ra complexes
ALT-803 (Altor)

| | | |
|---|---|---|
| SEQ ID NO: | IL-15N72D | NWVNVISDLKKIEDLIQSMHIDATLYTESDVHPSCKVTA |
| 1003 | | MKCFLLELQVISLESGDASIHDTVENLIILANDSLSSNGN |
| | | VTESGCKECEELEEKNIKEFLQSFVHIVQMFINTS |
| SEQ ID NO: | IL-15RaSu/ Fc | ITCPPPMSVEHADIWVKSYSLYSRERYICNSGFKRKAGTSSLTECVL |
| 1004 | | NKATNVAHWTTPSLKCIREPKSCDKTHTCPPCPAPELLGGPSVFLFP |
| | | PKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSV |
| | | MHEALHNHYTQKSLSLSPGK |

IL-15 / IL-15Ra sushi domain fusion (Cytune)

| | | |
|---|---|---|
| SEQ ID | Human IL-15 | NWVNVISDLKKIEDLIQSMHIDATLYTESDVHPSCKVTAMKCFLLE |
| NO: 1005 | | LQVISLESGDASIHDTVENLIILANNSLSSNGNVTESGCKECEELEXK |
| | | NIKEFLQSFVHIVQMFINTS |
| | | Where X is E or K |
| SEQ ID | Human IL-15Ra | ITCPPPMSVEHADIWVKSYSLYSRERYICNSGFKRKAGTSSLTECVL |
| NO: 1006 | sushi and hinge | NKATNVAHWTTPSLKCIRDPALVHQRPAPP |
| | domains | |

### CTLA-4 Inhibitors

In one aspect of the invention, the IL-1β inhibitor or a functional fragment thereof is administered together with an inhibitor of CTLA-4. In some embodiments, the CTLA-4 inhibitor is an anti-CTLA-4 antibody or fragment thereof. Exemplary anti-CTLA-4 antibodies include Tremelimumab (formerly ticilimumab, CP-675,206); and Ipilimumab (MDX-010, Yervoy®).

In one embodiment, the present invention provides an IL-1β antibody or a functional fragment thereof (e.g., canakinumab or gevokizumab) for use in the treatment of cancers having at least partial inflammatory bases, e.g., lung cancer, especially NSCLC, wherein said IL-1β antibody or a functional fragment thereof is administered in combination with one or more chemotherapeutic agent, wherein said one or more chemotherapeutic agent is a check point inhibitor, preferably selected from the group consisting of nivolumab, pembrolizumab, atezolizumab, avelumab, durvalumab, PDR-001 (spartalizumab) and Ipilimumab. In one embodiment the one or more chemotherapeutic agent is a PD-1 or PD-L-1 inhibitor, preferably selected from the group consisting of nivolumab, pembrolizumab, atezolizumab, avelumab, durvalumab, PDR-001 (spartalizumab), further preferably pembrolizumab. In one further embodiment, the IL-1β antibody or a functional fragment thereof is administered at the same time of the PD-1 or PD-L1 inhibitor.

In one embodiment the cancer of the patient has has high PD-L1 expression. Typically high PD-L1 expression is defined as Tumor Proportion Score (TPS) equal or greater than about 50%, as determined by an FDA-approved test.

In one embodiment said patient has a tumor that has high PD-L1 expression [Tumor Proportion Score (TPS)≥50%)] as determined by an FDA-approved test, with or without EGFR or ALK genomic tumor aberrations. In one embodiment said patient has tumor that has PD-L1 expression (TPS≥1%) as determined by an FDA-approved test.

The term “in combination with” is understood as the two or more drugs are administered subsequently or simultaneously. Alternatively, the term “in combination with” is understood that two or more drugs are administered in the manner that the effective therapeutic concentrations of the drugs are expected to be overlapping for a majority of the period of time within the patient's body. The DRUG of the invention and one or more combination partner (e.g., another drug, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or “used in combination” or “administered in combination” or the like as utilized herein are meant to encompass administration of the selected combination partner to a single subject in need thereof (e.g., a patient), and are intended to include treatment regimens in which the agents are not necessarily administered by the same route of administration or at the same time. The drug administered to a patient as separate entities either simultaneously, concurrently or sequentially with no specific time limits, wherein such administration provides therapeutically effective levels of the two compounds in the body of the patient and the treatment regimen will provide beneficial effects of the drug combination in treating the conditions or disorders described herein. The latter also applies to cocktail therapy, e.g., the administration of three or more active ingredients.

### Administration, Formulations and Devices

Canakinumab can be administered intravenously or preferably subcutaneously. Both administration routes are applicable to each and every canakinumab related embodiments disclosed in this application unless in embodiments wherein the administration route is specified.

Gevokizumab can be administered subcutaneously or preferably intravenously. Both administration routes are applicable to each and every gevokizumab related embodiments disclosed in this application unless in embodiments wherein the administration route is specified.

Canakinumab can be prepared as a medicament in a lyophilized form for reconstitution. In one embodiment canakinumab is provided in the form of lyophilized form for reconstitution containing at least about 200 mg drug per vial, preferably not more than about 250 mg, preferably not more than about 225 mg in one vial.

In one aspect the present invention provides canakinumab or gevokizumab for use in treating and/or preventing a cancer in a patient in need thereof, comprising administering a therapeutically effective amount to the patient, wherein the cancer has at least a partial inflammatory basis, and wherein canakinumab or gevokizumab is administered by a prefilled syringe or by an auto-injector. Preferably the prefilled syringe or the auto-injector contains the full amount of therapeutically effective amount of the drug. Preferably the prefilled syringe or the auto-injector contains about 200 mg of canakinumab.

### Efficacy and Safety

Due to its good safety profile, canakinumab or gevokizumab can be administered to a patient for a long period of time, providing and maintaining the benefit of suppressing IL-1β mediated inflammation. Furthermore due to its anti-cancer effect, either used in monotherapy or in combination with one or more therapeutic agents, patients' lives can be extended, including but not limited to extended duration of DFS, PFS, OS, hazard risk reduction, than without the Treatment of the Invention. The term “Treatment of the Invention”, as used in the this application, refers to DRUG of the invention, suitably canakinumab or gevokizumab, administered according to the dosing regimen, as taught in this application. Preferably the clinical efficacy is achieved at a dose of about 200 mg canakinumab administered about every 3 weeks or about monthly, preferably for at least about 6 months, preferably at least about 12 months, preferably at least about 24 months, preferably up to about 2 years, preferably up to about 3 years. Preferably the results is achieved at a dose of about 30 mg-120 mg gevokizumab administered about every 3 weeks or about monthly, preferably for at least about 6 months, preferably at least about 12 months, preferably at least about 24 months, preferably up to about 2 years, preferably up to about 3 years. In one embodiment Treatment of the Invention is the sole treatment. In one embodiment Treatment of the Invention is added on top of the SoC treatment for the cancer indication. While the SoC treatment evolves with time, the SoC treatment as used here should be understood as not including DRUG of the invention.

Thus in one aspect the present invention provides an IL-1β binding antibody or functional fragment thereof, suitably canakinumab or gevokizumab, for use in the treatment of MDS in a patient, wherein a therapeutically effective amount of an IL-1β binding antibody or a functional fragment thereof is administered to the patient for at least about 6 months, preferably at least about 12 months, preferably at least about 24 months.

In one aspect, the present invention provides an IL-1β binding antibody or functional fragment thereof, suitably canakinumab or gevokizumab, for use in the treatment of MDS in a patient, wherein the hazard risk of cancer mortality of the patient is reduced by at least about 10%, at least about 20%, at least about 30%, at least about 40% or at least about 50%, preferably compared to not receiving Treatment of the Invention.

The term “not receiving Treatment of the Invention”, as used throughout the application, include patients who did not receive any drug at all and patients who received only treatment, considered as SoC at the time, without the DRUG of the invention. As a skilled person would understand, the clinical efficacy is typically not tested within the same patient, receiving or not receiving the Treatment of the Invention, rather tested in clinical trial settings with treatment group and placebo group.

In one embodiment the overall survival (OS, defined as the time from the date of randomization to the date of death due to any cause) of the patient is at least about one month, at least about 3 months, at least about 6 months, at least about 12 months longer compared to not receiving Treatment of the Invention. In one embodiment the OS is at least about 12 months, preferably at least about 24 months, longer in the adjuvant treatment setting. In one embodiment the OS is at least about 4 months, preferably at least about 6 months, or at least about 12 months longer in the first line treatment setting. In one embodiment the OS is at least about one month, at least about 3 months, or preferably at least about 6 months longer in the 2^{nd}/3^{rd} line treatment setting.

In one embodiment the overall survival in the patient receiving Treatment of the Invention is at least about 2 years, at least about 3 years, at least about 5 years, at least about 8 years, or at least about 10 years in the adjuvant treatment setting. In one embodiment the overall survival in the patient receiving Treatment of the Invention is at least about 6 months, at least about one year, or at least about 3 years in the first line treatment setting. In one embodiment the overall survival in the patient receiving Treatment of the Invention is at least about 3 months, at least about 6 months, or at least about one year in the 2^{nd}/3^{rd} line treatment setting.

In one embodiment the progression free survival (PFS) period of the patient receiving Treatment of the Invention is extended by at least about one month, at least about 2 months, at least about 3 months, at least about 6 months, or at least about 12 months, preferably compared to not receiving Treatment of the Invention. In one embodiment PFS is extended by at least about 6 months, preferably at least about 12 months in the first line treatment settings. In one embodiment PFS is extended by at least about one month, at least about 3 months, or at least about 6 months in the second line treatment settings.

In one embodiment the patient receiving Treatment of the Invention has at least about 3 months, at least about 6 months, at least about 12 months, or at least about 24 months progression free survival.

Normally clinical efficacy, including but not limited to DFS, PFS, HR reduction, OS, can be demonstrated in clinical trials comparing a treatment group and a placebo group. In the placebo group patients receive no drug at all or receive SoC treatment. In the treatment group patients receive DRUG of the invention either as monotherapy or added to the SoC treatment. Alternatively in the placebo group patients receive SoC treatment and in the treatment group patients receive DRUG of the invention.

Even though the clinical outcome, such as duration of DFS or the HR reduction of cancer mortality, is described as a number based on statistical analysis of a clinical trial, one of ordinary skill would readily extrapolate these statistics to treatments for an individual patient, as claimed, since it is expected the DRUG of the invention would achieve a similar clinical outcome in a portion of the individual patients receiving Treatment of the Invention, for example in about 95% of the patients, when clinical trials have demonstrated statistical significance (p≤0.05)); or for example in about 50% of the patients, when clinical trials have provided a mean value, such as mean PFS being about 24 months. IL-1β blockade could affect patients' immune system when combating an infection. Thus in one aspect the present invention provides an IL-1β binding antibody or a functional fragment thereof, suitably canakinumab or gevokizumab, for use in the treatment and/or prevention of cancer, e.g., cancer having at least a partial inflammatory basis, and wherein the patient is not at high risk of developing a serious infection due to the Treatment of the Invention. The patient would be at high risk of developing a serious infection due to the Treatment of the Invention in the following situations, but not limited to those situations: (a) Patients have an active infection requiring medical intervention. The term “active infection requiring medical intervention” is understood as the patient is currently taking or has been taking or has just finished taking for less than about one month or less than about two weeks, any anti-viral and/or any anti-bacterial medicines; (b) Patients have latent tuberculosis and/or a history of tuberculosis.

To manage the inhibition of the immune system by IL-1β blockade, it is cautioned that the IL-1β binding antibody or a functional fragment thereof is not administered concomitantly with a TNF inhibitor. Preferably a TNF inhibitor is selected from a group consisting of Enbrel® (etanercept), Humira® (adalimumab), Remicade® (infliximab), Simponi® (golimumab), and Cimzia® (certolizumab pegol). It is also cautioned that the IL-1β binding antibody or a functional fragment thereof is not administered concomitantly with another IL-1 blocker, wherein preferably said IL-1 blocker is selected from a group consisting of Kineret® (anakinra) and Arcalyst® (rilonacept). Furthermore it is only one IL-1β binding antibody or a functional fragment thereof is administered in the treatment/prevention of cancer. For example canakinumab is not administered in combination with gevokizumab.

When canakinumab is administered to patients, it is likely that some patients will develop anti-canakinumab antibodies (anti-drug antibody, ADA), which needs to be monitored for safety and efficacy reasons. In one aspect the present invention provides canakinumab for use in the treatment and/or the prevention of cancer, e.g., cancer having at least a partial inflammatory basis, wherein the chance of the patient develops ADA is less than about 1%, less than about 0.7%, less than about 0.5%, less than about 0.4%. In one embodiment the antibody is detected by the method as described in EXAMPLE 10. In one embodiment the antibody detection is performed at about 3 months, at about 6 months, or at about 12 months from the first administration of canakinumab.

### Cancer to be Treated According to the Present Invention

In one aspect, the present invention provides an IL-1β binding antibody or a functional fragment thereof, suitably gevokizumab or suitably canakinumab, alone or in combination with one or more therapeutic agents, for use in the treatment of cancer, e.g., cancer having at least partial inflammatory basis, wherein said cancer includes myelodysplastic syndromes (MDS), suitably low risk MDS, or wherein said cancer includes other myeloid neoplasms such as chronic myelomonocytic leukemia (CMML), myeloproliferative neoplasms (MPN), and multiple myeloma (MM).

In one aspect, the present invention provides an IL-1β binding antibody or a functional fragment thereof, suitably gevokizumab or suitably canakinumab, alone or in combination with one or more therapeutic agents, for use in the treatment of myelodysplastic syndromes (MDS), suitably low risk MDS. In one embodiment, the present invention provides an IL-1β binding antibody or a functional fragment thereof, suitably gevokizumab or suitably canakinumab, alone or in combination with one or more therapeutic agents, for use in the treatment of anemia in myelodysplastic syndromes (MDS), suitably anemia in low risk MDS.

Myelodysplastic syndromes (MDS) are a group of cancers characterized by impaired peripheral blood cell production (cytopenias) and most commonly a hypercellular, dysplastic-appearing bone marrow. The MDS are diseases of the hematopoietic stem cells. They are characterized by disturbances of differentiation and maturation, and by changes in the bone marrow stroma. Diagnostic criteria have been set up to diagnose the MDS: 2 classification systems (French-American-British [FAB] and World Health Organization [WHO]) and several prognostic-scoring systems, the most common being the International Prognostic Scoring System (IPSS) (Nimer, Blood, 2008, Germing et al., Dtsch Arztebl Int. 2013). There is also a revised version of the IPSS, called the Revised International Prognostic Scoring System (IPSS-R) for myelodysplastic syndromes. It has been developed by the International Working Group for Prognosis in MDS (IWG). It can be used via the IPSS-R Calculator at https://www.mds-foundation.org/ipss-r-calculator/.

The IWG also defined response criteria to standardize response evaluation for clinical decision-making as well as comparison of clinical trial data across studies. One of those response criteria is the hematologic improvement-erythroid (HI-E). The response criteria were recently revised (Platzbecker et al., Blood (20 19) 133 (10): 1020-1030). Transfusion dependency and haemoglobin levels are parameters of the HI-E response.

Myelodysplastic syndromes (MDS) are currently classified by the WHO as shown in the following Table.

TABLE 11
WHO classification of myelodysplastic syndromes (Brunning et al. and Orazi et al.
In: WHO classification of tumours of haematopoietic and lymphoid tissues, 2008)

| MDS subtype | Blood | Bone marrow |
|---|---|---|
| Refractory cytopenia with | <1% blasts | <5% blasts |
| unilineage dysplasia (RCUD) | Uni- or bicytopenia | Dysplasia in ≥10% of cells |
| Refractory anemia (RA) | | of only one lineage |
| Refractory neutropenia (RN) | | |
| Refractory thrombocytopenia (RT) | | |
| Refractory anemia with | Anemia, no blasts | <5% blasts, ≥15% ring |
| ring sideroblasts (RARS) | | sideroblasts within |
| | | erythropoiesis, exclusively |
| | | dyserythropoiesis |
| Refractory cytopenia with | <1% blasts | <5% blasts, signs of |
| multilineage dysplasia (RCMD) | Cytopenia | dysplasia in ≥10% of cells |
| with or without ring sideroblasts | <1000/μl monocytes | of 2 to 3 lineages |
| MDS with isolated del(5q) | <1% blasts | Mostly typical mononuclear |
| | Anemia, platelets | megakaryocytes, <5% blasts, |
| | often increased | isolated del(5q) abnormality |
| Refractory anemia with | Cytopenia <5% blasts | Uni- or multilineage |
| excess of blasts I (RAEB I) | <1000/μl monocytes | dysplasia, blasts 5% to 9%, |
| | | no Auer rods |
| Refractory anemia with | Cytopenia <20% blasts | Uni- or multilineage |
| excess of blasts II (RAEB II) | <1000/μl monocytes | dysplasia, blasts 10% to 19%, |
| | Auer rods may be found | Auer rods may be found |
| Unclassified MDS (MDS-U) | <1% blasts | <5% blasts |
| RCUD with pancytopenia | <1000/μl monocytes | |
| RCMD/RCUD with 1% blasts in | | |
| blood | | |
| MDS-typical chromosomal | | |
| aberration without clear | | |
| signs of dysplasia | | |

According to the assessment of patient risk, level of anemia, and presence of del(5q) chromosomal or cytogenetic abnormalities, the term “myelodysplastic syndromes” or “MDS” includes three groups of patients: “low risk patients without del(5q) chromosomal/cytogenetic abnormalities and Epo<500 mU/mL”, “low risk patients without del(5q) chromosomal/cytogenetic abnormalities and Epo>500 mU/mL”, and “higher risk patients”. The level of patient risk is quantified using the International prognostic scoring system (IPSS and revised IPSS-R) and/or the WHO prognostic scoring system (WPSS). Low risk is defined as: IPSS Low, Intermediate-1; IPSS-R very low, low, intermediated; or WPSS very low, low, intermediate. Higher risk is defined as: IPSS Intermediate-2, High; IPSS-R Intermediate, High, Very High; or WPSS High, Very High. Additional genetic biomarkers can be used to identify patients in a low risk category, which would benefit from a treatment that is normally only given to high risk patients.

In one embodiment, the MDS patient is transfusion dependent.

In one embodiment, the MDS patient has an anemia.

Since chronic inflammation is implicated in the development of MDS (Barreyro et al., Blood. 2018, Basiorka et al., Blood. 2016; 128(25):2960-2975, Yin et al., Life Sci. 2016; 165:109-112) it's possible that DRUG of the invention, preferably canakinumab or gevokizumab, could be combined with current standard of care in low risk patient groups independent of their background chromosomal/cytogenetic profile or Epo level at the first stages of presentation.

Since IL-1β has been directly implicated in suppressing erythropoietin expression (Cluzeau et al., Haematologica. 2017; 102(12): 2015-2020), DRUG of the invention, preferably canakinumab or gevokizumab, could be useful therapies in those patients with low Epo.

In one embodiment, the present invention provides DRUG of the invention, preferably canakinumab or gevokizumab, for use in the treatment of MDS, wherein DRUG of the invention is administered in combination with one or more therapeutic agents.

In one embodiment the one or more therapeutic agents is selected from erythropoiesis stimulating agents (ESA), including erythropoietin, epoetin alfa, epoetin beta, epoetin omega, epoetin delta, epoetin zeta, epoetin theta, darbepoetin alfa, methoxy polyethylene glycol-epoetin beta; granulocyte-colony stimulating factor (G-CSF); lenalidomide; azacitidine (AzaC); decitabine; thrombopoietin receptor agonists (TPO) including avatrombopag, eltrombopag, lusutrombopag, promegapoietin, romiplostim, thrombopoietin; and chemotherapeutic agents suitable for intensive induction chemotherapy. In one embodiment, the one or more chemotherapeutic agents is alpelisib. Alpelisib is administered at a therapeutically effective amount of about 300 mg per day. In one embodiment, the one or more chemotherapeutic agents is eltrombopag. Eltrombopag is administered at a therapeutically effective amount of about 75 mg per day.

Depending on the patient condition, one, two or three of the therapeutic agents can be selected from the above lists to be combined with DRUG of the invention.

In one embodiment the one or more therapeutic agents is the standard of care (SoC) agent for MDS. In one preferred embodiment the one or more therapeutic agents is AzaC. In one preferred embodiment the therapeutic one or more therapeutic agents is decitabine. In one embodiment the one or more therapeutic agents is a lenalidomide. In one embodiment the one or more therapeutic agents is a ESA with or without G-CSF.

In one embodiment the one or more therapeutic agents is a HDM2-p53 interaction inhibitor, e.g., (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201, WO 2013/111105, example 102) or a pharmaceutically acceptable non-covalent derivative (including salt, solvate, hydrate, complex, co-crystal) thereof, preferably a succinic acid derivative, e.g., succinic acid co-crystal (e.g., crystalline Form B, prepared according to method D in WO 2013/111105, page 392).

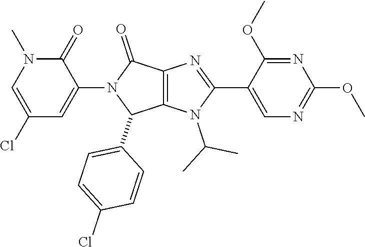 [SMILES: COc1ncc(-c2nc3c(n2C(C)C)[C@H](c2ccc(Cl)cc2)N(c2cc(Cl)cn(C)c2=O)C3=O)c(OC)n1]

In one embodiment DRUG of the invention is used in the MDS treatment in combination with an immunosuppressive therapy or hematopoietic cell transplantation.

In one embodiment DRUG of the invention is used in the MDS treatment in combination with one or more therapeutic agents, further in combination with immunosuppressive therapy or hematopoietic cell transplantation. Immunosuppressive therapy can be done with anti-thymocyte globulin (ATG) with or without cyclosporine.

While patients await a suitable donor match for hematopoietic cell transplantation, canakinumab or gevokizumab could be utilized in combination with intensive induction chemotherapy for patients eligible for intensive induction chemotherapy, or as a potential combination partner to either AzaC or decitabine in those patients not eligible for intensive induction chemotherapy.

In one embodiment, DRUG of the invention, preferably canakinumab or gevokizumab is used in combination with MBG453.

In one embodiment, DRUG of the invention, preferably canakinumab or gevokizumab is used in combination with luspatercept.

In one embodiment, DRUG of the invention, preferably canakinumab or gevokizumab is used, alone or preferably in combination with one or more therapeutic agents, in the first line treatment of MDS. In one embodiment the one or more therapeutic agents is a therapeutic agent used as first line treatment selected from an ESA, including erythropoietin, epoetin alfa, epoetin beta, epoetin omega, epoetin delta, epoetin zeta, epoetin theta, darbepoetin alfa, methoxy polyethylene glycol-epoetin beta; G-CSF; AzaC; decitabine; or lenalidomide. In one embodiment the one or more therapeutic agent is ESA with or without G-CSF. In one embodiment the one or more therapeutic agent is AzaC, decitabine, or lenalidomide. In one embodiment immunosuppressive therapy or hematopoietic cell transplantation is given instead of or in addition to the one or more therapeutic agents.

Preferably DRUG of the invention is used in combination with one or more therapeutic agents with the SoC drugs, which are approved as the first line treatment of MDS, for example an ESA, including erythropoietin, epoetin alfa, epoetin beta, epoetin omega, epoetin delta, epoetin zeta, epoetin theta, darbepoetin alfa, methoxy polyethylene glycol-epoetin beta, with or without G-CSF, or AzaC, decitabine, or lenalidomide.

In one embodiment, DRUG of the invention, preferably canakinumab or gevokizumab is used, alone or preferably in combination with one or more therapeutic agent, in second or third line treatment of MDS. In one embodiment one or more therapeutic agents is s ESA+lenalidomide with or without G-CSF. In one embodiment one or more therapeutic agents is TPO.

In one embodiment, DRUG of the invention, preferably canakinumab or gevokizumab is used as a second line treatment of MDS after therapy with an ESA, including erythropoietin, epoetin alfa, epoetin beta, epoetin omega, epoetin delta, epoetin zeta, epoetin theta, darbepoetin alfa, methoxy polyethylene glycol-epoetin beta; G-CSF; AzaC; decitabine; lenalidomide; or luspatercept.

In one embodiment, DRUG of the invention, preferably canakinumab or gevokizumab, is used as second line treatment of MDS after treatment with luspatercept.

All the uses disclosed throughout this application, including but not limited to, doses and dosing regimens, combinations, route of administration and biomarkers can be applied to the treatment of MDS.

The word “a” and “an” have been generally defined as “at least one” or “one or more” in the specification.

The word “patient” refers to human patient.

The following Examples illustrate the invention described above; they are not, however, intended to limit the scope of the invention in any way.

### EXAMPLES

The Examples below is set forth to aid in the understanding of the invention but is not intended, and should not be construed, to limit its scope in any way.

### Example 1

### Tumor-Derived IL-1β Induces Differential Tumor Promoting Mechanisms in Metastasis Materials and Methods

### Cell Culture

Human breast cancer MDA-MB-231-Luc2-TdTomato (Calliper Life Sciences, Manchester UK), MDA-MB-231 (parental) MCF7, T47D (European Collection of Authenticated Cell Cultures (ECACC)), MDA-MB-231-IV (Nutter et al., 2014) as well as bone marrow HS5 (ECACC) and human primary osteoblasts OB1 were cultured in DMEM+10% FCS (Gibco, Invitrogen, Paisley, UK). All cell lines were cultured in a humidified incubator under 5% C02 and used at low passage >20.

### Transfection of Tumor Cells

Human MDA-MB-231, MCF 7 and T47D cells were stably transfected to overexpress genes IL1B or IL1R1 using plasmid DNA purified from competent E. coli that have been transduced with an ORF plasmid containing human IL1B or IL1R1 (Accession numbers NM_000576 and NM_0008777.2, respectively) with a C-terminal GFP tag (OriGene Technologies Inc. Rockville Md.). Plasmid DNA purification was performed using a PureLink™ HiPure Plasmid Miniprep Kit (ThemoFisher) and DNA quantified by UV spectroscopy before being introduced into human cells with the aid of Lipofectamine II (ThermoFisher). Control cells were transfected with DNA isolated from the same plasmid without IL-1β or IL-1R1 encoding sequences.

### In Vitro Studies

In vitro studies were carried out with and without addition of 0-5 ng/ml recombinant IL-1β (R&D systems, Wiesbaden, Germany)+/−50 μM IL-1Ra (Amgen, Cambridge, UK).

Cells were transferred into fresh media with 10% or 1% FCS. Cell proliferation was monitored every 24 h for up to 120 h by manual cell counting using a 1/400 mm² hemocytometer (Hawkley, Lancing UK) or over a 72 h period using an Xcelligence RTCA DP Instrument (Acea Biosciences, Inc). Tumor cell invasion was assessed using 6 mm transwell plates with an 8 μm pore size (Corning Inc) with or without basement membrane (20% Matrigel; Invitrogen). Tumor cells were seeded into the inner chamber at a density of 2.5×10⁵ for parental as well as MDA-MB-231 derivatives and 5×10⁵ for T47D in DMEM+1% FCS and 5×10⁵ OB1 osteoblast cells supplemented with 5% FCS were added to the outer chamber. Cells were removed from the top surface of the membrane 24 h and 48 h after seeding and cells that had invaded through the pores were stained with hematoxylin and eosin (H&E) before being imaged on a Leica DM7900 light microscope and manually counted.

Migration of cells was investigated by analyzing wound closure: Cells were seeded onto 0.2% gelatine in 6-well tissue culture plates (Costar; Corning, Inc) and, once confluent, 10 μg/ml mitomycin C was added to inhibit cell proliferation and a 50 μm scratch made across the monolayer. The percentage of wound closure was measured at 24 h and 48 h using a CTR7000 inverted microscope and LAS-AF v2.1.1 software (Leica Applications Suite; Leica Microsystems, Wetzlar, Germany). All proliferation, invasion and migration experiments were repeated using the Xcelligence RTCA DP instrument and RCTA Software (Acea Biosystems, Inc).

For co-culture studies with human bone 5×10⁵ MDA-MB-231 or T47D cells were seeded onto tissue culture plastic or into 0.5 cm³ human bone discs for 24 h. Media was removed and analysed for concentration of IL-1β by ELISA. For co-culture with HS5 or OB1 cells, 1×10⁵ MDA-MB-231 or T47D cells were cultured onto plastic along with 2×10⁵ HS5 or OB1 cells. Cells were sorted by FACS 24 h later and counted and lysed for analysis of IL-1β concentration. Cells were collected, sorted and counted every 24 h for 120 h.

### Animals

Experiments using human bone grafts were carried out in 10-week old female NOD SCID mice. In IL-1P/IL-1R1 overexpression bone homing experiments 6 to 8-week old female BALB/c nude mice were used. To investigate effects of IL-1β on the bone microenvironment 10-week old female C57BL/6 mice (Charles River, Kent, UK) or IL-1R1/mice (Abdulaal et al., 2016) were used. Mice were maintained on a 12 h:12 h light/dark cycle with free access to food and water. Experiments were carried out with UK home office approval under project licence 40/3531, University of Sheffield, UK.

### Patient Consent and Preparation of Bone Discs

All patients provided written, informed consent prior to participation in this study. Human bone samples were collected under HTA licence 12182, Sheffield Musculoskeletal Biobank, University of Sheffield, UK. Trabecular bone cores were prepared from the femoral heads of female patients undergoing hip replacement surgery using an Isomat 4000 Precision saw (Buehler) with Precision diamond wafering blade (Buehler). 5 mm diameter discs were subsequently cut using a bone trephine before storing in sterile PBS at ambient temperature.

### In Vivo Studies

To model human breast cancer metastasis to human bone implants two human bone discs were implanted subcutaneously into 10-week old female NOD SCID mice (n=10/group) under isofluorane anaesthetic. Mice received an injection of 0.003 mg vetergesic and Septrin was added to the drinking water for 1 week following bone implantation. Mice were left for 4 weeks before injecting 1×10⁵ MDA-MB-231 Luc2-TdTomato, MCF7 Luc2 or T47D Luc2 cells in 20% Martigel/79% PBS/1% toluene blue into the two hind mammary fat pads. Primary tumor growth and development of metastases was monitored weekly using an IVIS (Luminol) system (Caliper Life Sciences) following sub-cutaneous injection of 30 mg/ml D-luciferin (Invitrogen). On termination of experiments mammary tumors, circulating tumor cells, serum and bone metastases were resected. RNA was processed for downstream analysis by real time PCR, and cell lysates were taken for protein analysis and whole tissue for histology as previously described (Nutter et al., 2014; Ottewell et al., 2014a).

For therapeutic studies in NOD SCID mice, placebo (control), 1 mg/kg IL-1Rα (Anakinra®) daily or 10 mg/kg canakinumab subcutaneously every 14 days were administered starting 7 days after injection of tumor cells. In BALB/c mice and C57BL/6 mice 1 mg/kg IL-1Ra was administered daily for 21 or 31 days or 10 mg/kg canakinumab was administered as a single subcutaneous injection. Tumor cells, serum, and bone were subsequently resected for downstream analysis.

Bone metastases were investigated following injection of 5×10⁵ MDA-MB-231 GFP (control), MDA-MB-231-IV, MDA-MB-231-IL-1B-positive or MDA-MB-231-IL-1R1-positive cells into the lateral tail vein of 6 to 8-week old female BALB/c nude mice (n=12/group). Tumor growth in bones and lungs was monitored weekly by GFP imaging in live animals. Mice were culled 28 days after tumor cell injection at which timepoint hind limbs, lungs and serum were resected and processed for microcomputed tomography imaging (μCT), histology and ELISA analysis of bone turnover markers and circulating cytokines as described (Holen et al., 2016).

### Isolation of Circulating Tumor Cells

Whole blood was centrifuged at 10,000 g for 5 minutes and the serum removed for ELISA assays. The cell pellet was re-suspended in 5 ml of FSM lysis solution (Sigma-Aldrich, Pool, UK) to lyse red blood cells. Remaining cells were re-pelleted, washed 3× in PBS and re-suspended in a solution of PBS/10% FCS. Samples from 10 mice per group were pooled prior to isolation of TdTomato positive tumor cells using a MoFlow High performance cell sorter (Beckman Coulter, Cambridge UK) with the 470 nM laser line from a Coherent I-90C tenable argon ion (Coherent, Santa Clara, Calif.). TdTomato fluorescence was detected by a 555LP dichroic long pass and a 580/30 nm band pass filter. Acquisition and analysis of cells was performed using Summit 4.3 software. Following sorting cells were immediately placed in RNA protect cell reagent (Ambion, Paisley, Renfrew, UK) and stored at −80° C. before RNA extraction. For counting numbers of circulating tumor cells, TdTomato fluorescence was detected using a 561 nm laser and an YL1-A filter (585/16 emission filter). Acquisition and analysis of cells was performed using Attune NxT software.

### Microcomputed Tomography Imaging

Microcomputed tomography (CT) analysis was carried out using a Skyscan 1172 x-ray-computed CT scanner (Skyscan, Aartselar, Belgium) equipped with an x-ray tube (voltage, 49 kV; current, 200 uA) and a 0.5-mm aluminium filter. Pixel size was set to 5.86 μm and scanning initiated from the top of the proximal tibia as previously described (Ottewell et al., 2008a; Ottewell et al., 2008b).

### Bone Histology and Measurement of Tumor Volume

Bone tumor areas were measured on three non-serial, H&E stained, 5 μm histological sections of decalcified tibiae per mouse using a Leica RMRB upright microscope and Osteomeasure software (Osteometrics, Inc. Decauter, USA) and a computerised image analysis system as previously described (Ottewell et al., 2008a).

### Western Blotting

Protein was extracted using a mammalian cell lysis kit (Sigma-Aldrich, Poole, UK). 30 μg of protein was run on 4-15% precast polyacrylamide gels (BioRad, Watford, UK) and transferred onto an Immobilon nitrocellulose membrane (Millipore). Non-specific binding was blocked with 1% casein (Vector Laboratories) before incubation with rabbit monoclonal antibodies to human N-cadherin (D4R1H) at a dilution of 1:1000, E-cadherin (24E10) at a dilution of 1:500 or gamma-catenin (2303) at a dilution of 1:500 (Cell signalling) or mouse monoclonal GAPDH (ab8245) at a dilution of 1:1000 (AbCam, Cambridge UK) for 16 h at 4° C. Secondary antibodies were anti-rabbit or anti-mouse horse radish peroxidase (HRP; 1:15,000) and HRP was detected with the Supersignal chemiluminescence detection kit (Pierce). Band quantification was carried out using Quantity Once software (BioRad) and normalised to GAPDH.

### Gene Analysis

Total RNA was extracted using an RNeasy kit (Qiagen) and reverse transcribed into cDNA using Superscript III (Invitrogen AB). Relative mRNA expression of IL-1B (Hs02786624), IL-1R1 (Hs00174097), CASP (Caspase 1) (Hs00354836), IL1RN (Hs00893626), JUP (junction plakoglobin/gamma-catenin) (Hs00984034), N-cadherin (Hs01566408) and E-cadherin (Hs1013933) were compared with the housekeeping gene glyceraldehyde-3-phosphate dehydrogenase (GAPDH; Hs02786624) and assessed using an ABI 7900 PCR System (Perkin Elmer, Foster City, Calif.) and Taqman universal master mix (Thermofisher, UK). Fold change in gene expression between treatment groups was analysed by inserting CT values into Data Assist V3.01 software (Applied Biosystems) and changes in gene expression were only analysed for genes with a CT value of ≤25.

Assessment of IL-1β and IL-1R1 in Tumors from Breast Cancer Patients

IL-1β and IL-1R1 expression was assessed on tissue microarrays (TMA) containing primary breast tumor cores taken from 1,300 patients included in the clinical trial, AZURE (Coleman et al. 2011). Samples were taken pre-treatment from patients with stage II and III breast cancer without evidence of metastasis. Patients were subsequently randomized to standard adjuvant therapy with or without the addition of zoledronic acid for 10 years (Coleman et al 2011). The TMAs were stained for IL-1β (ab2105, 1:200 dilution, Abcam) and IL-1R1 (ab59995, 1:25 dilution, Abcam) and scored blindly under the guidance of a histopathologist for IL-1β/IL-1R1 in the tumor cells or in the associated stroma. Tumor or stromal IL-1β or IL-1R1 was then linked to disease recurrence (any site) or disease recurrence specifically in bone (+/− other sites).

### The IL-1β Pathway is Upregulated During the Process of Human Breast Cancer Metastasis to Human Bone.

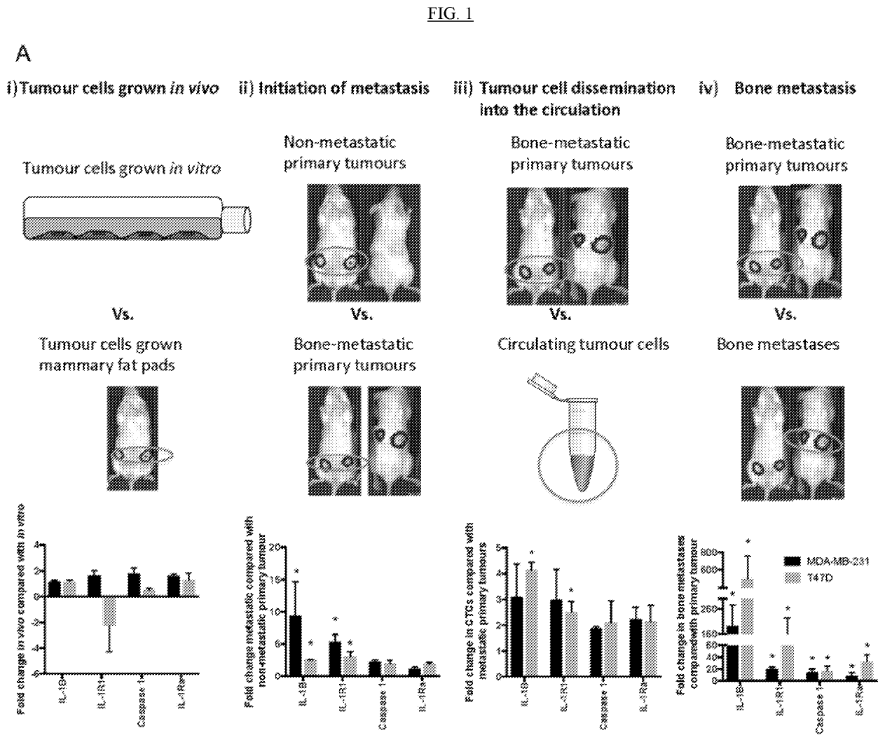

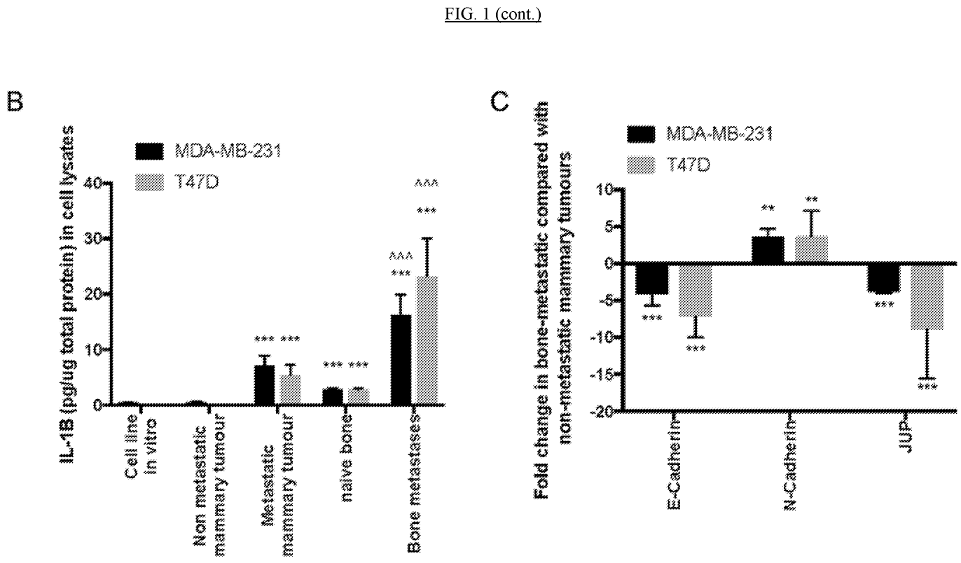

A mouse model of spontaneous human breast cancer metastasis to human bone implants was utilised to investigate how the IL-1β pathway changes through the different stages of metastasis. Using this model, the expression levels of genes associated with the IL-1β pathway increased in a stepwise manner at each stage of the metastatic process in both triple negative (MDA-MB-231) and estrogen receptor positive (ER+ve) (T47D) breast cancer cells: Genes associated with the IL-1β signalling pathway (IL-1B, IL-1R1, CASP (Caspase 1) and IL-1Ra) were expressed at very low levels in both MDA-MB-231 and T47D cells grown in vitro and expression of these genes were not altered in primary mammary tumors from the same cells that did not metastasize in vivo (FIG. 1a).

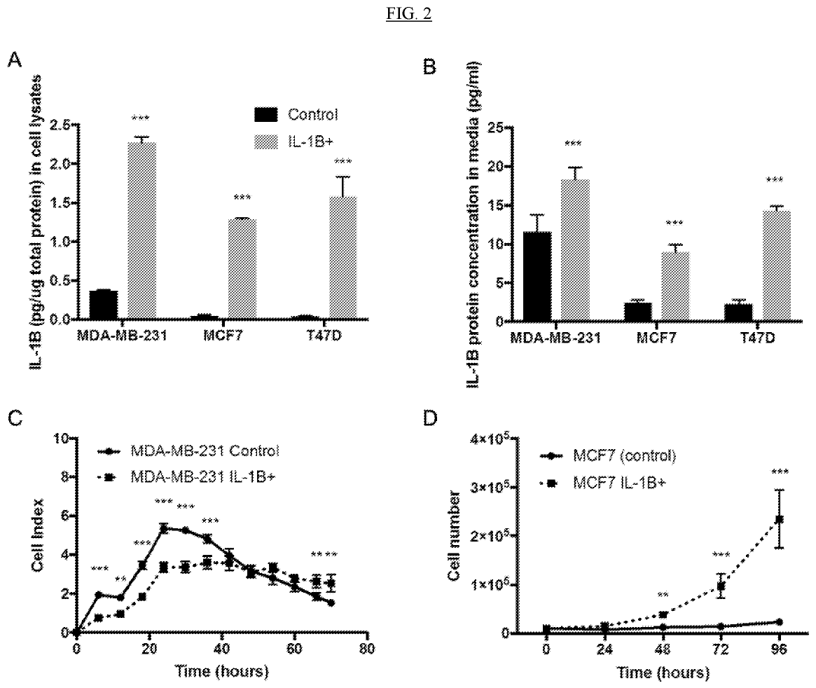

IL-1B, IL-1R1 and CASP were all significantly increased in mammary tumors that subsequently metastasized to human bone compared with those that did not metastasize (p<0.01 for both cell lines), leading to activation of IL-1β signalling as shown by ELISA for the active 17 kD IL-1β (FIG. 1b; FIG. 2). IL-1B gene expression increased in circulating tumor cells compared with metastatic mammary tumors (p<0.01 for both cell lines) and IL-1B (p<0.001), IL-1R1 (p<0.01), CASP (p<0.001) and IL-1Rα (p<0.01) were further increased in tumor cells isolated from metastases in human bone compared with their corresponding mammary tumors, leading to further activation of IL-1β protein (FIG. 1; FIG. 2). These data suggest that IL-1β signalling may promote both initiation of metastasis from the primary site as well as development of breast cancer metastases in bone.

### Tumor Derived IL-1β Promotes EMT and Breast Cancer Metastasis.

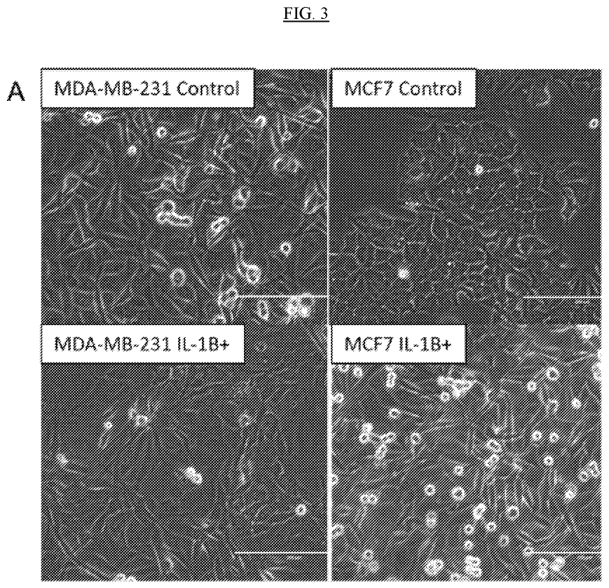

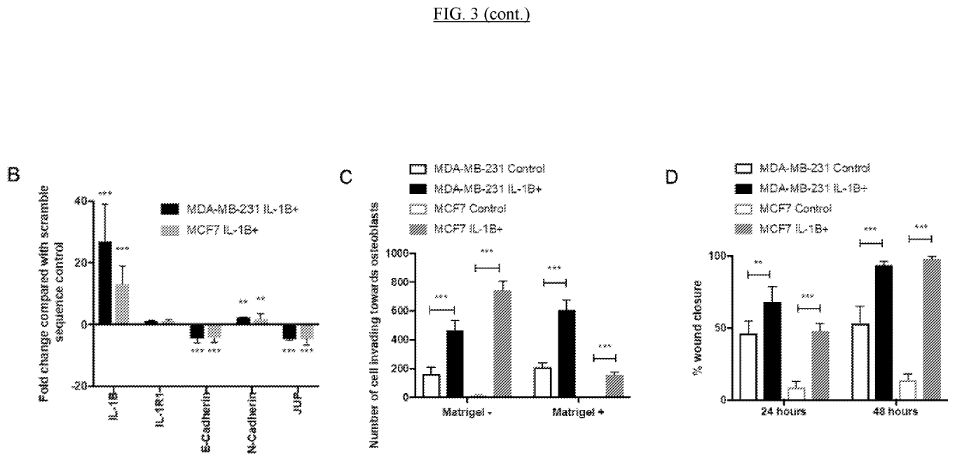

Expression levels of genes associated with tumor cell adhesion and epithelial to mesenchymal transition (EMT) were significantly altered in primary tumors that metastasised to bone compared with tumors that did not metastasise (FIG. 1c). IL-1β-overexpressing cells were generated (MDA-MB-231-IL-1B+, T47D-IL-1B+ and MCF7-IL-1B+) to investigate whether tumor-derived IL-1β is responsible for inducing EMT and metastasis to bone. All IL-1β+ cell lines demonstrated increased EMT exhibiting morphological changes from an epithelial to mesenchymal phenotype (FIG. 3a) as well as reduced expression of E-cadherin, and JUP (junction plakoglobin/gamma-catenin) and increased expression of N-Cadherin gene and protein (FIG. 3b). Wound closure (p<0.0001 in MDA-MB-231-IL-10+(FIG. 3d); p<0.001 MCF7-IL-1β+ and T47D-IL-1β+) and migration and invasion through matrigel towards osteoblasts were increased in tumor cells with increased IL-1β signalling compared with their respective controls (MDA-MB-231-IL-1β+(FIG. 3c) p<0.0001; MCF7-IL-1β+ and T47D-IL-1β+p<0.001). Increased IL-1β production was seen in ER-positive and ER-negative breast cancer cells that spontaneously metastasized to human bone implants in vivo compared with non-metastatic breast cancer cells (FIG. 1). The same link between IL-1β and metastasis was made in primary tumor samples from patients with stage II and III breast cancer enrolled in the AZURE study (Coleman et al., 2011) that experienced cancer relapsed over a 10 year time period. IL-1β expression in primary tumors from the AZURE patients correlated with both relapse in bone and relapse at any site indicating that presence of this cytokine is likely to play a role in metastasis in general. In agreement with this, genetic manipulation of breast cancer cells to artificially overexpress IL-1β increased the migration and invasion capacities of breast cancer cells in vitro (FIG. 3).

### Inhibition of IL-1β Signaling Reduces Spontaneous Metastasis to Human Bone.

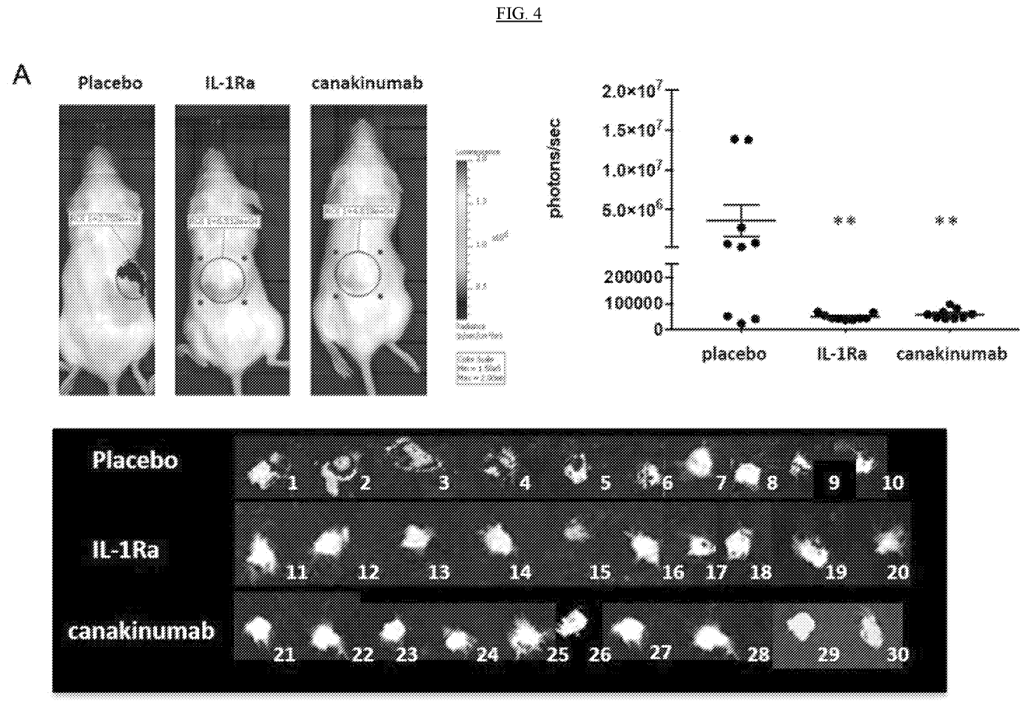

As tumor derived IL-1β appeared to be promoting onset of metastasis through induction of EMT the effects of inhibiting IL-1β signaling with IL-1Ra (Anakinra) or a human anti-IL-1β-binding antibody (canakinumab) on spontaneous metastasis to human bone implants were investigated: Both IL-1Ra and canakinumab reduced metastasis to human bone: metastasis was detected in human bone implants in 7 out of 10 control mice, but only in 4 out of 10 mice treated with IL-1Ra and 1 out of 10 mice treated with canakinumab. Bone metastases from IL-1Ra and canakinumab treatment groups were also smaller than those detected in the control group (FIG. 4a). Numbers of cells detected in the circulation of mice treated with canakinumab or IL-1Ra were significantly lower than those detected in the placebo treated group: only 3 tumor cells/ml were counted in whole blood from mice treated with canakinumab and anakinra, respectively, compared 108 tumor cells/ml counted in blood from placebo treated mice (FIG. 4b), suggesting that inhibition of IL-1 signalling prevents tumor cells from being shed from the primary site into the circulation. Therefore, inhibition of IL-1β signaling with the anti-IL-1β antibody canakinumab or inhibition of IL-1R1 reduced the number of breast cancer cells shed into the circulation and reduced metastases in human bone implants (FIG. 4).

### Tumor Derived IL-1B Promotes Bone Homing and Colonisation of Breast Cancer Cells.

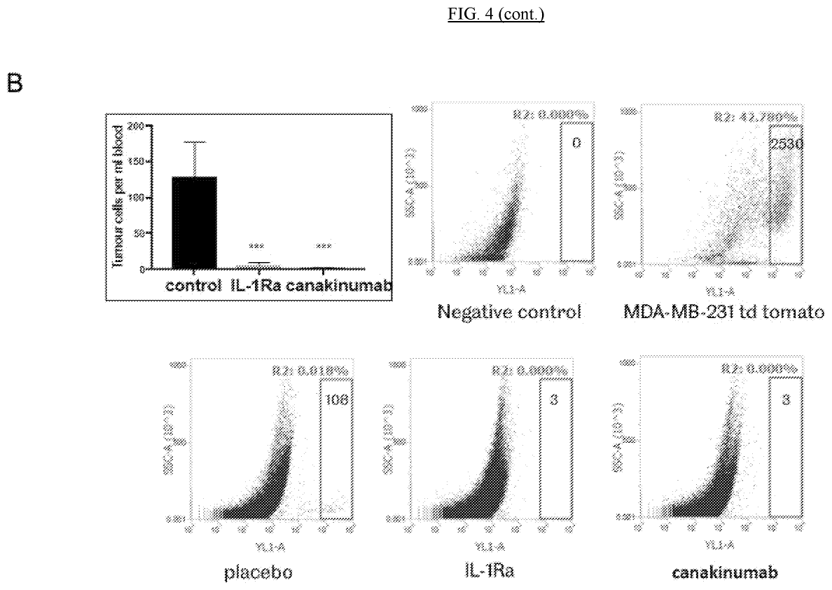

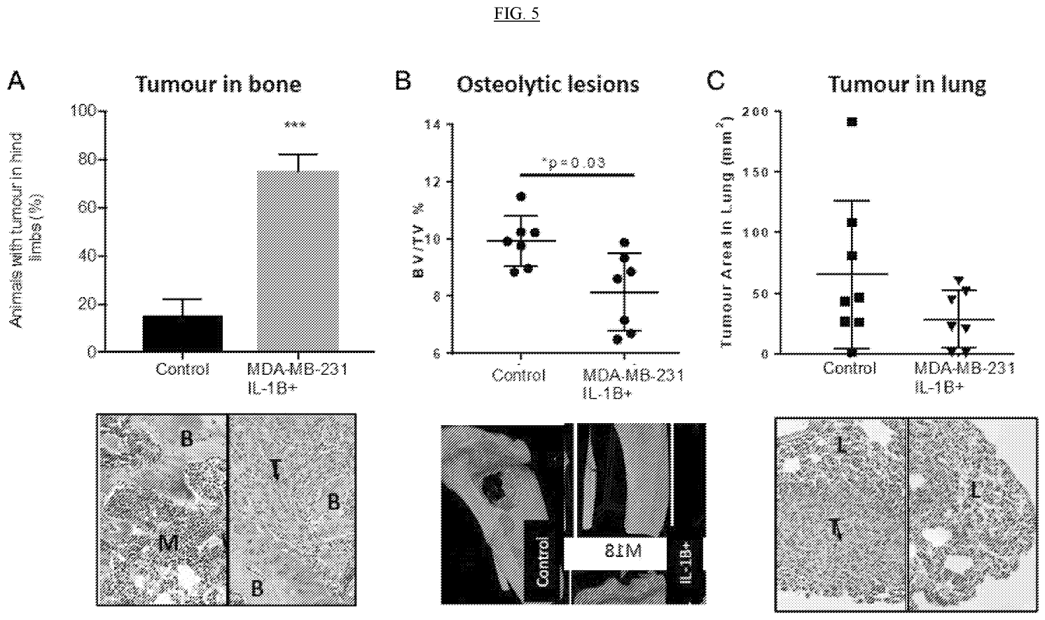

Injection of breast cancer cells into the tail vein of mice usually results in lung metastasis due to the tumor cells becoming trapped in the lung capillaries. It was previously shown that breast cancer cells that preferentially home to the bone microenvironment following intra-venous injection express high levels of IL-1β, suggesting that this cytokine may be involved in tissue specific homing of breast cancer cells to bone. In the current study, intravenous injection of MDA-MB-231-IL-1β+ cells into BALB/c nude mice resulted in significantly increased number of animals developing bone metastasis (75%) compared with control cells (12%) (p<0.001) cells (FIG. 5a). MDA-MB-231-IL-1β+ tumors caused development of significantly larger osteolytic lesions in mouse bone compared with control cells (p=0.03; FIG. 5b) and there was a trend towards fewer lung metastases in mice injected with MDA-MB-231-IL-1β+ cells compared with control cells (p=0.16; FIG. 5c). These data suggest that endogenous IL-1β can promote tumor cell homing to the bone environment and development of metastases at this site.

### Tumor Cell-Bone Cell Interactions Further Induce IL-1B and Promote Development of Overt Metastases.

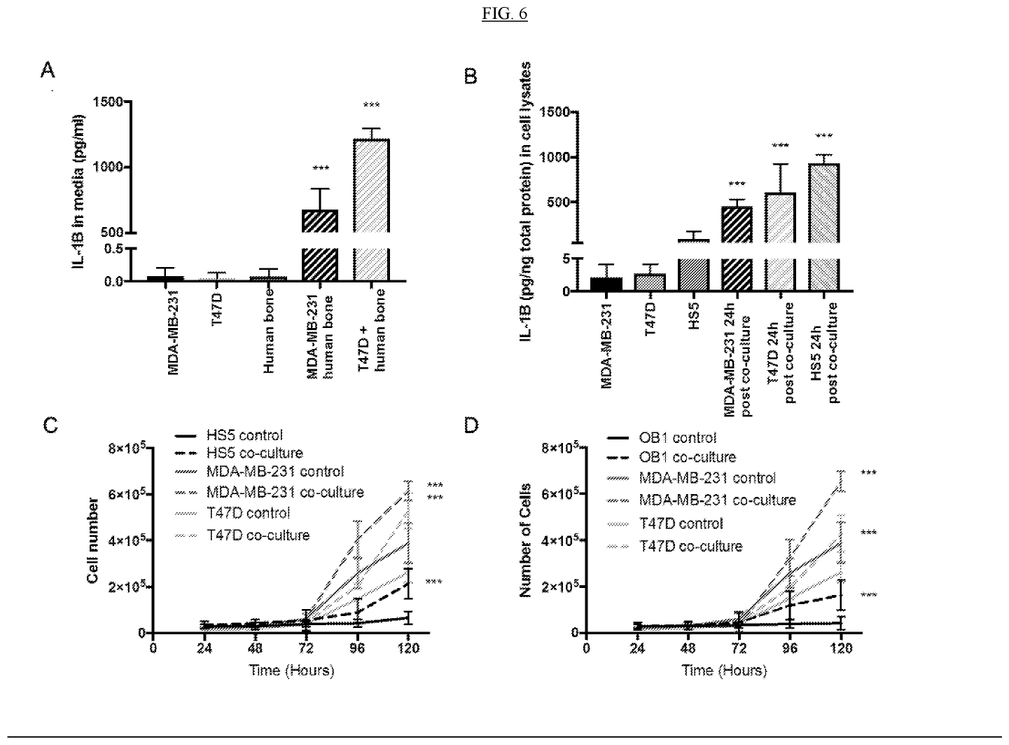

Gene analysis data from a mouse model of human breast cancer metastasis to human bone implants suggested that the IL-1β pathway was further increased when breast cancer cells are growing in the bone environment compared with metastatic cells in the primary site or in the circulation (FIG. 1a). It was therefore investigated how IL-1β production changes when tumor cells come into contact with bone cells and how IL-1β alters the bone microenvironment to affect tumor growth (FIG. 6). Culture of human breast cancer cells into pieces of whole human bone for 48 h resulted in increased secretion of IL-1β into the medium (p<0.0001 for MDA-MB-231 and T47D cells; FIG. 6a). Co-culture with human HS5 bone marrow cells revealed the increased IL-1βB concentrations originated from both the cancer cells (p<0.001) and bone marrow cells (p<0.001), with IL-1β from tumor cells increasing ˜1000 fold and IL-1B from HS5 cells increasing ˜100 fold following co-culture (FIG. 6b).

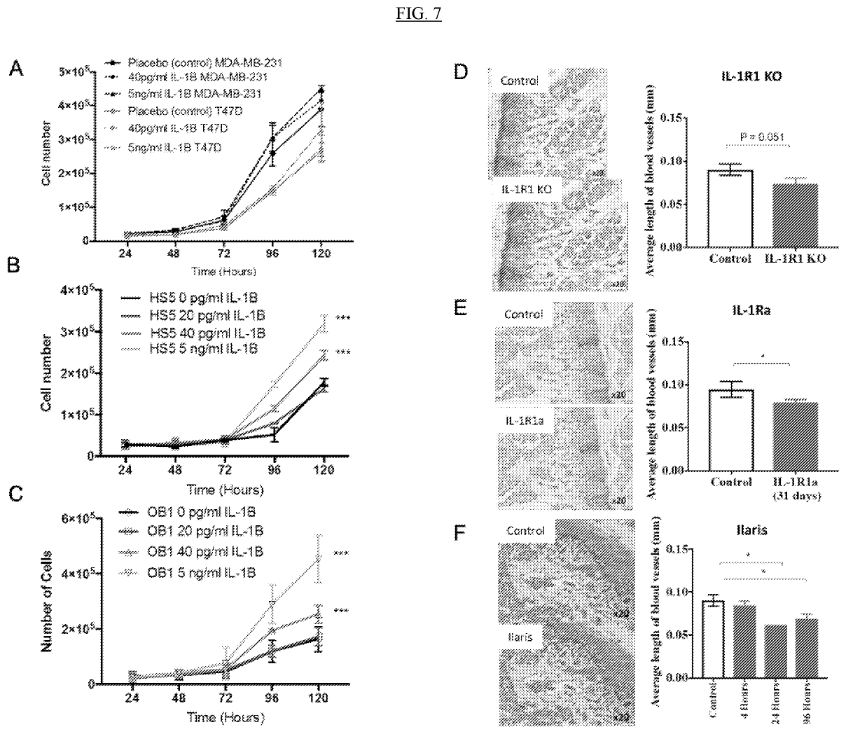

Exogenous IL-1β did not increase tumor cell proliferation, even in cells overexpressing IL-1R1. Instead, IL-1β stimulated proliferation of bone marrow cells, osteoblasts and blood vessels that in turn induced proliferation of tumor cells (FIG. 6). It is therefore likely that arrival of tumor cells expressing high concentrations of IL-1β stimulate expansion of the metastatic niche components and contact between IL-1β expressing tumor cells and osteoblasts/blood vessels drive tumor colonization of bone. The effects of exogenous IL-1β as well as IL-1β from tumor cells on proliferation of tumor cells, osteoblasts, bone marrow cells and CD34⁺ blood vessels were investigated: Co-culture of HS5 bone marrow or OB1 primary osteoblast cells with breast cancer cells caused increased proliferation of all cell types (P<0.001 for HS5, MDA-MB-231 or T47D, FIG. 6c) (P<0.001 for OB1, MDA-MB-231 or T47D, FIG. 6). Direct contact between tumor cells, primary human bone samples, bone marrow cells or osteoblasts promoted release of IL-1β from both tumor and bone cells (FIG. 6). Furthermore, administration of IL-1β increased proliferation of HS5 or OB1 cells but not breast cancer cells (FIG. 7a-c), suggesting that tumor cell-bone cell interactions promote production of IL-1β that can drive expansion of the niche and stimulate the formation of overt metastases.

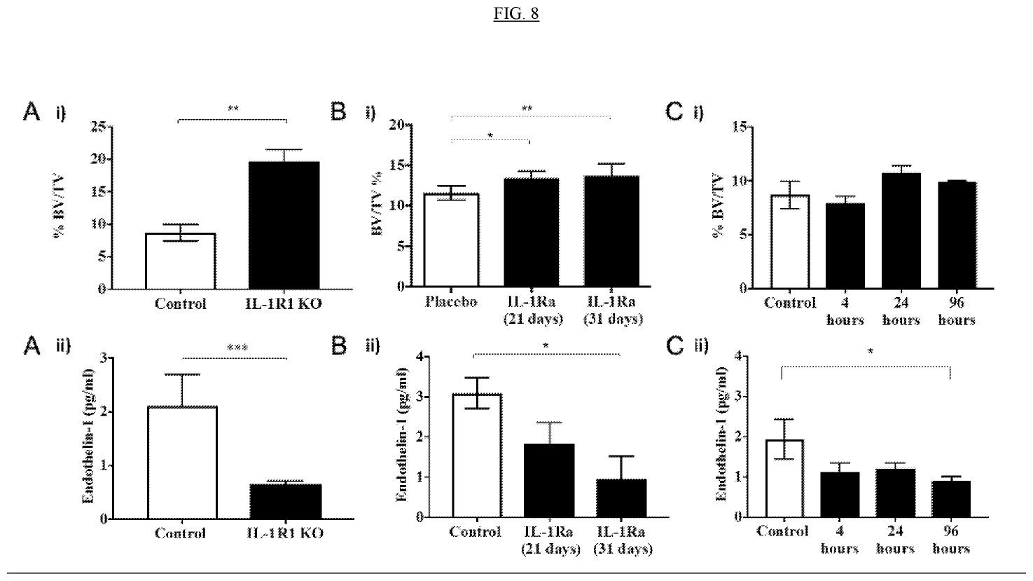

IL-1β signalling was also found to have profound effects on the bone microvasculature: Preventing IL-1β signaling in bone by knocking out IL-1R1, pharmacological blockade of IL-1R with IL-1Rα or reducing circulating concentrations of IL-1β by administering the anti-IL-1β binding antibody canakinumab reduced the average length of CD34⁺ blood vessels in trabecular bone, where tumor colonisation takes place (p<0.01 for IL-1Rα and canakinumab treated mice) (FIG. 7 c). These findings were confirmed by endomeucin staining which showed decreased numbers of blood vessels as well as blood vessel length in bone when IL-1β signaling was disrupted. ELISA analysis for endothelin 1 and VEGF showed reduced concentrations of both of these endothelial cell markers in the bone marrow for IL-1R1^{-/-} mice (p<0.001 endothelin 1; p<0.001 VEGF) and mice treated with IL-1R antagonist (p<0.01 endothlin 1; p<0.01 VEGF) or canakinumab (p<0.01 endothelin 1; p<0.001 VEGF) compared with control (FIG. 8). These data suggest that tumor cell-bone cell associated increases in IL-1β and high levels of IL-1β in tumor cells may also promote angiogenesis, further stimulating metastases.

### Tumor Derived IL-1β Predicts Future Breast Cancer Relapse in Bone and Other Organs in Patient Material

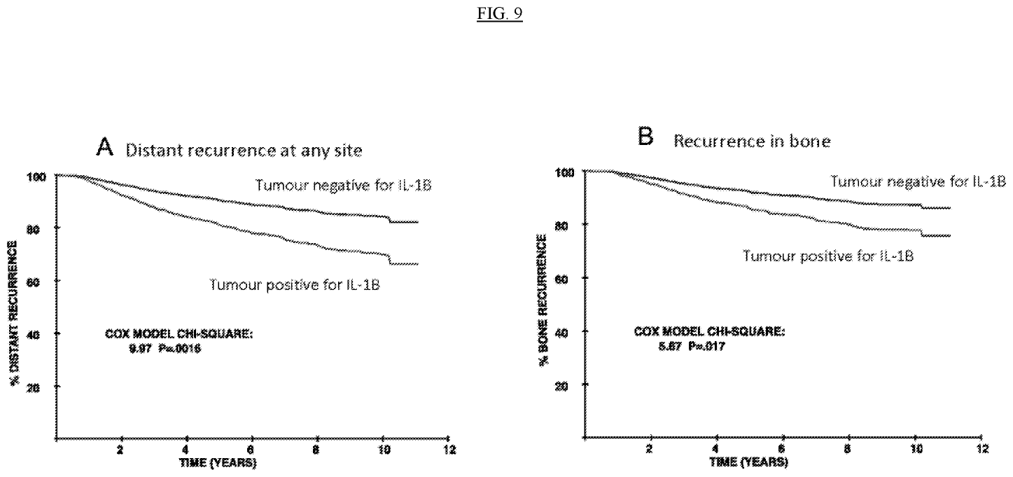

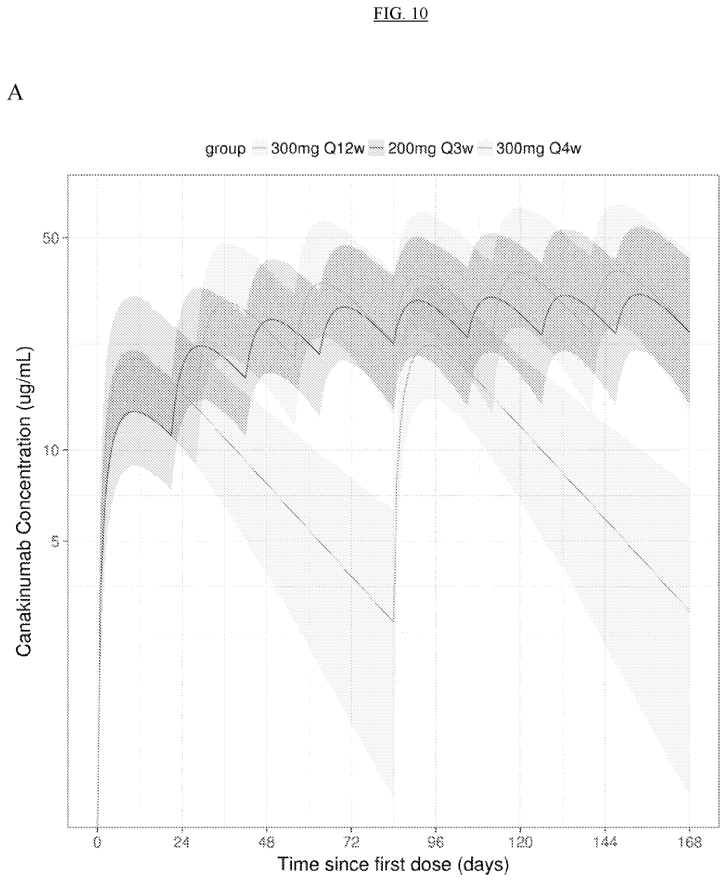

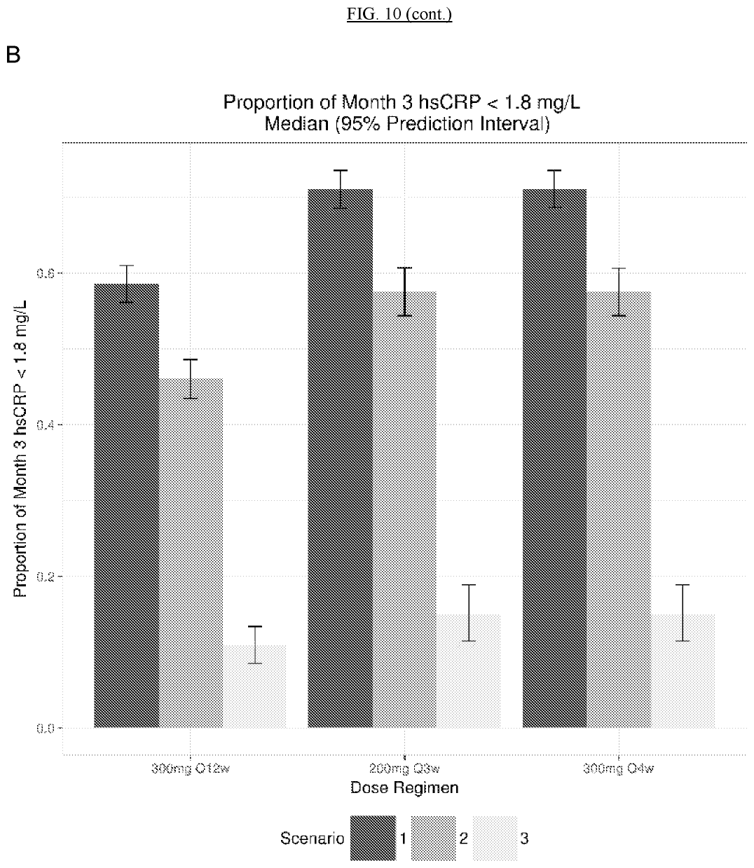

To establish the relevance of the findings in a clinical setting the correlation between IL-1β and its receptor IL-1R1 in patient samples was investigated. ˜1300 primary tumor samples from patients with stage II/III breast cancer with no evidence of metastasis (from the AZURE study (Coleman et al., 2011)) were stained for IL-1R1 or the active (17 kD) form of IL-1β, and biopsies were scored separately for expression of these molecules in the tumor cells and the tumor associated stroma. Patients were followed up for 10 years following biopsy and correlation between IL-1β/IL-1R1 expression and distant recurrence or relapse in bone assessed using a multivariate Cox model. IL-1β in tumor cells strongly correlated with distant recurrence at any site (p=0.0016), recurrence only in bone (p=0.017) or recurrence in bone at any time (p=0.0387) (FIG. 9). Patients who had IL-1β in their tumor cells and IL-1R1 in the tumor associated stroma were more likely to experience future relapse at a distant site (p=0.042) compared to patients who did not have IL-1β in their tumor cells, indicating that tumor derived IL-1β may not only promote metastasis directly but may also interact with IL-1R1 in the stroma to promote this process. Therefore, IL-1β is a novel biomarker that can be used to predict risk of breast cancer relapse.

### Example 2

Simulation of Canakinumab PK Profile and hsCRP Profile for Lung Cancer Patients.

A model was generated to characterize the relationship between canakinumab pharmacokinetics (PK) and hsCRP based on data from the CANTOS study.

The following methods were used in this study: Model building was performed using the first-order conditional estimation with interaction method. The model described the logarithm of the time resolved hsCRP as:

y(tᵢⱼ)=y_{0,i}+y_{eff}(tᵢⱼ)

where y_{0,i} is a steady state value and y_{eff}(tᵢⱼ) describes the effect of the treatment and depends on the systemic exposure. The treatment effect was described by an Emax-type model,

$${y}_{eff} ( {t}_{ij} ) = {E}_{max , i} \frac{c ( {t}_{ij} )}{c ( {t}_{ij} ) + IC {50}_{i}}$$

where E_{max,i} is the maximal possible response at high exposure, and IC50ᵢ is the concentration at which half maximal response is obtained.

The individual parameters, E_{max,i} and y_{0,i} and the logarithm of IC50ᵢ were estimated as a sum of a typical value, covariate effects covpar*covᵢ and normally distributed between subject variability. In the term for the covariate effect covpar refers to the covariate effect parameter being estimated and covᵢ is the value of the covariate of subject i. Covariates to be included were selected based on inspection of the eta plots versus covariates. The residual error was described as a combination of proportional and additive term.

The logarithm of baseline hsCRP was included as covariate on all three parameters (E_{max,i}, y_{0,i} and IC50ᵢ). No other covariate was included into the model. All parameters were estimated with good precision. The effect of the logarithm of the baseline hsCRP on the steady state value was less than 1 (equal to 0.67). This indicates that the baseline hsCRP is an imperfect measure for the steady state value, and that the steady state value exposes regression to the mean relative to the baseline value. The effects of the logarithm of the baseline hsCRP on IC50 and Emax were both negative. Thus patients with high hsCRP at baseline are expected to have low IC50 and large maximal reductions. In general, model diagnostics confirmed that the model describes the available hsCRP data well.

The model was then used to simulate expected hsCRP response for a selection of different dosing regimens in a lung cancer patient population. Bootstrapping was applied to construct populations with intended inclusion/exclusion criteria that represent potential lung cancer patient populations. Three different lung cancer patient populations described by baseline hsCRP distribution alone were investigated: all CANTOS patients (scenario 1), confirmed lung cancer patients (scenario 2), and advanced lung cancer patients (scenario 3).

The population parameters and inter-patient variability of the model were assumed to be the same for all three scenarios. The PK/PD relationship on hsCRP observed in the overall CANTOS population was assumed to be representative for lung cancer patients.

The estimator of interest was the probability of hsCRP at end of month 3 being below a cut point, which could be either 2 mg/L or 1.8 mg/L. 1.8 mg/L was the median of hsCRP level at end of month 3 in the CANTOS study. Baseline hsCRP >2 mg/L was one of the inclusion criteria, so it is worthy to explore if hsCRP level at end of month 3 went below 2 mg/L.

A one-compartment model with first order absorption and elimination was established for CANTOS PK data. The model was expressed as ordinary differential equation and RxODE was used to simulate canakinumab concentration time course given individual PK parameters. The subcutaneous canakinumab dose regimens of interest were 300 mg Q12W, 200 mg Q3W, and 300 mg Q4W. Exposure metrics including Cmin, Cmax, AUCs over different selected time periods, and average concentration Cave at steady state were derived from simulated concentration time profiles.

The simulation in Scenario 1 was based on the below information:

Individual canakinumab exposure simulated using RxODE

PD parameters which are components of y_{0,i}, E_{max,i}, and IC50: typical values (THETA(3), THETA(5), THETA(6)), covpars (THETA(4), THETA(7), THETA(8)), and between subject variability (ETA(1), ETA(2), ETA(3))

Baseline hsCRP from all 10,059 CANTOS study patients (baseline hsCRP: mean 6.18 mg/L, standard error of the mean (SEM)=0.10 mg/L)

The prediction interval of the estimator of interest was produced by first randomly sampling 1000 THETA(3)-(8)s from a normal distribution with fixed mean and standard deviation estimated from the population PK/PD model; and then for each set of THETA(3)-(8), bootstrapping 2000 PK exposure, PD parameters ETA(1)-(3), and baseline hsCRP from all CANTOS patients. The 2.5%, 50%, and 97.5% percentile of 1000 estimates were reported as point estimator as well as 95% prediction interval.

The simulation in Scenario 2 was based on the below information:

Individual canakinumab PK exposure simulated using RxODE

PD parameters THETA(3)-(8) and ETA(1)-(3)

Baseline hsCRP from 116 CANTOS patients with confirmed lung cancer (baseline hsCRP: mean=9.75 mg/L, SEM=1.14 mg/L)

The prediction interval of the estimator of interest was produced by first randomly sampling 1000 THETA(3)-(8)s from a normal distribution with fixed mean and standard deviation estimated from the population PKPD model; and then for each set of THETA(3)-(8), bootstrapping 2000 PK exposure, PD parameters ETA(1)-(3) from all CANTOS patients, and bootstrapping 2000 baseline hsCRP from the 116 CANTOS patients with confirmed lung cancer. The 2.5%, 50%, and 97.5% percentile of 1000 estimates were reported as point estimator as well as 95% prediction interval.

In Scenario 3, the point estimator and 95% prediction interval were obtained in a similar manner as for scenario 2. The only difference was bootstrapping 2000 baseline hsCRP values from advanced lung cancer population. There is no individual baseline hsCRP data published in an advanced lung cancer population. An available population level estimate in advanced lung cancer is a mean of baseline hsCRP of 23.94 mg/L with SEM 1.93 mg/L [Vaguliene 2011]. Using this estimate, the advanced lung cancer population was derived from the 116 CANTOS patients with confirmed lung cancer using an additive constant to adjust the mean value to 23.94 mg/L.

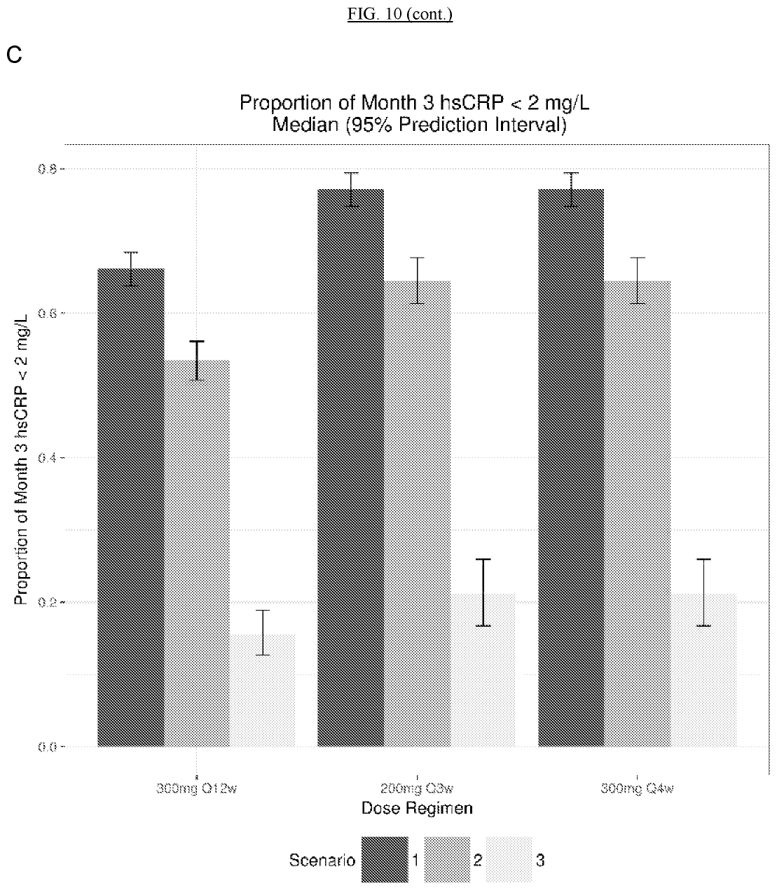

In line with the model, the simulated canakinumab PK was linear. The median and 95% prediction interval of concentration time profiles are plotted in natural logarithm scale over 6 months is shown in FIG. 10a.

The median and 95% prediction intervals of 1000 estimates of proportion of subjects with month 3 hsCRP response under the cut point of 1.8 mg/L and 2 mg/L mhsCRP are reported in FIGS. 10b and c. Judging from the simulation data, 200 mg Q3W and 300 mg Q4W perform similarly and better than 300 mg Q12W (top dosing regimen in CANTOS) in terms of decreasing hsCRP at month 3. Going from scenario 1 to scenario 3 towards more severe lung cancer patients, higher baseline hsCRP levels are assumed, and result in smaller probabilities of month 3 hsCRP being below the cut point. FIG. 10d shows how the median hsCRP concentration changes over time for three different doses and FIG. 10e shows the percent reduction from baseline hsCRP after a single dose.

### Example 3

### PDR001 Plus Canakinumab Treatment Increases Effector Neutrophils in Colorectal Tumors.

RNA sequencing was used to gain insights on the mechanism of action of canakinumab (ACZ885) in cancer. The CPDR001×2102 and CPDR001×2103 clinical trials evaluate the safety, tolerability and pharmacodynamics of spartalizumab (PDR001) in combination with additional therapies. For each patient, a tumor biopsy was obtained prior to treatment, as well as cycle 3 of treatment. In brief, samples were processed by RNA extraction, ribosomal RNA depletion, library construction and sequencing. Sequence reads were aligned by STAR to the hg19 reference genome and Refseq reference transcriptome, gene-level counts were compiled by HTSeq, and sample-level normalization using the trimmed mean of M-values was performed by edgeR.

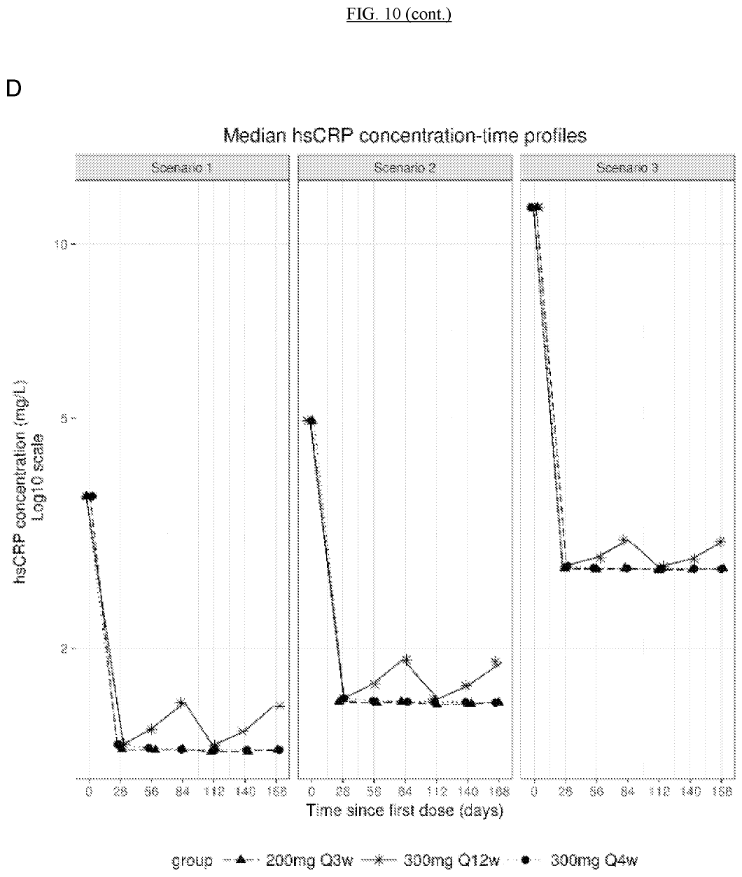

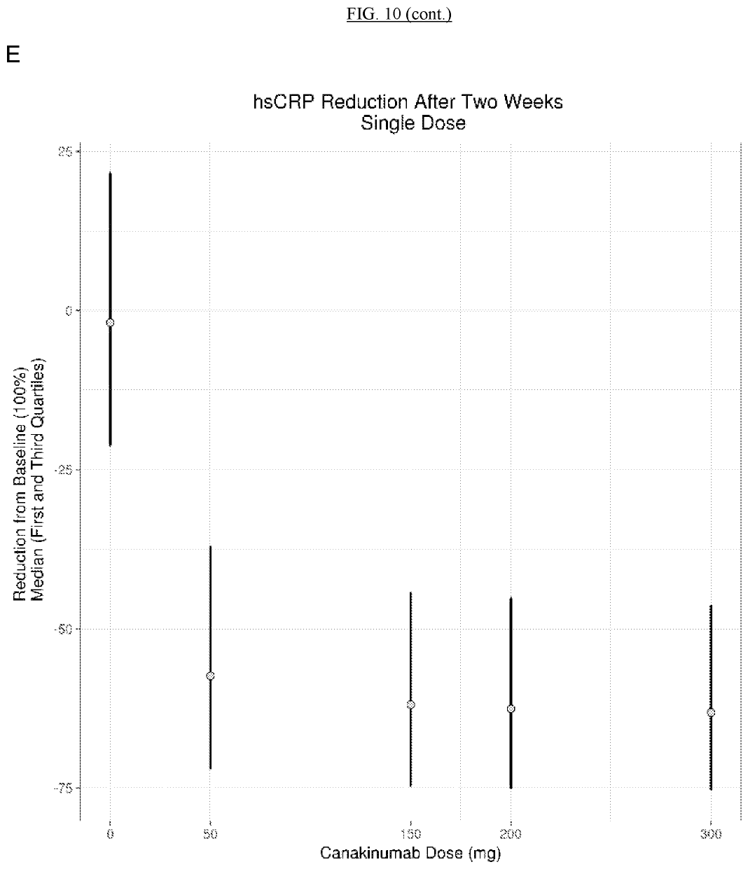

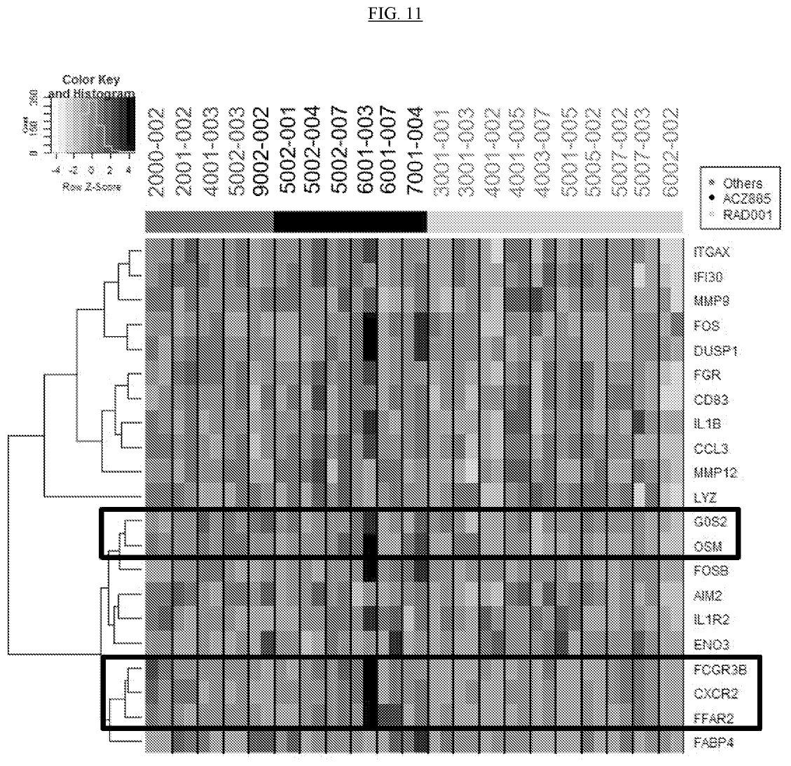

FIG. 11 shows 21 genes that were increased, on average, in colorectal tumors treated with PDR001+canakinumab (ACZ885), but not in colorectal tumors treated with PDR001+everolimus (RAD001). Treatment with PDR001+canakinumab increased the RNA levels of IL1B, as well as its receptor, IL1R2. This observation suggests an on-target compensatory feedback by tumors to increase IL1B RNA levels in response to IL-1β protein blockade.

Notably, several neutrophil-specific genes were increased on PDR001+canakinumab, including FCGR3B, CXCR2, FFAR2, OSM, and G0S2 (indicated by boxes in FIG. 11). The FCGR3B gene is a neutrophil-specific isoform of the CD16 protein. The protein encoded by FCGR3B plays a pivotal role in the secretion of reactive oxygen species in response to immune complexes, consistent with a function of effector neutrophils (Fossati G 2002 Arthritis Rheum 46: 1351). Chemokines that bind to CXCR2 mobilize neutrophils out of the bone marrow and into peripheral sites. In addition, increased CCL3 RNA was observed on treatment with PDR001+canakinumab. CCL3 is a chemoattractant for neutrophils (Reichel C A 2012 Blood 120: 880).

In summary, this contribution of components analysis using RNA-seq data demonstrates that PDR001+canakinumab treatment increases effector neutrophils in colorectal tumors, and that this increase was not observed with PDR001+everolimus treatment.

### Example 4

Efficacy of Canakinumab (ACZ885) in Combination with Spartalizumab (PDR001) in the Treatment of Cancer.

Patient 5002-004 is a 56 year old man with initially Stage IIC, microsatellite-stable, moderately differentiated adenocarcinoma of the ascending colon (MSS-CRC), diagnosed in June, 2012 and treated with prior regimens.

Prior treatment regimens included:

Folinic acid/5-fluoruracil/oxaliplatin in the adjuvant setting

Chemoradiation with capecitabine (metastatic setting)

5-fluorouracil/bevacizumab/folinic acid/irinotecan

trifluridine and tipiracil

Irinotecan

Oxaliplatin/5-fluorouracil

5-fluorouracil/bevacizumab/leucovorin

5-fluorouracil

At study entry the patient had extensive metastatic disease including multiple hepatic and bilateral lung metastases, and disease in paraesophageal lymph nodes, retroperitoneum and peritoneum.

The patient was treated with PDR001 400 mg every four weeks (Q4W) plus 100 mg every eight weeks (Q8W) ACZ885. The patient had stable disease for 6 months of therapy, then with substantial disease reduction and confirmed RECIST partial response to treatment at 10 months. The patient has subsequently developed progressive disease and the dose was increased to 300 mg and then to 600 mg.

### Example 5

### Calculations for Selecting the Dose for Gevokizumab for Cancer Patients.

Dose selection for gevokizumab in the treatment of cancer having at least partial inflammatory basis is based on the clinical effective dosings reveals by the CANTOS trial in combination with the available PK data of gevokizumab, taking into the consideration that

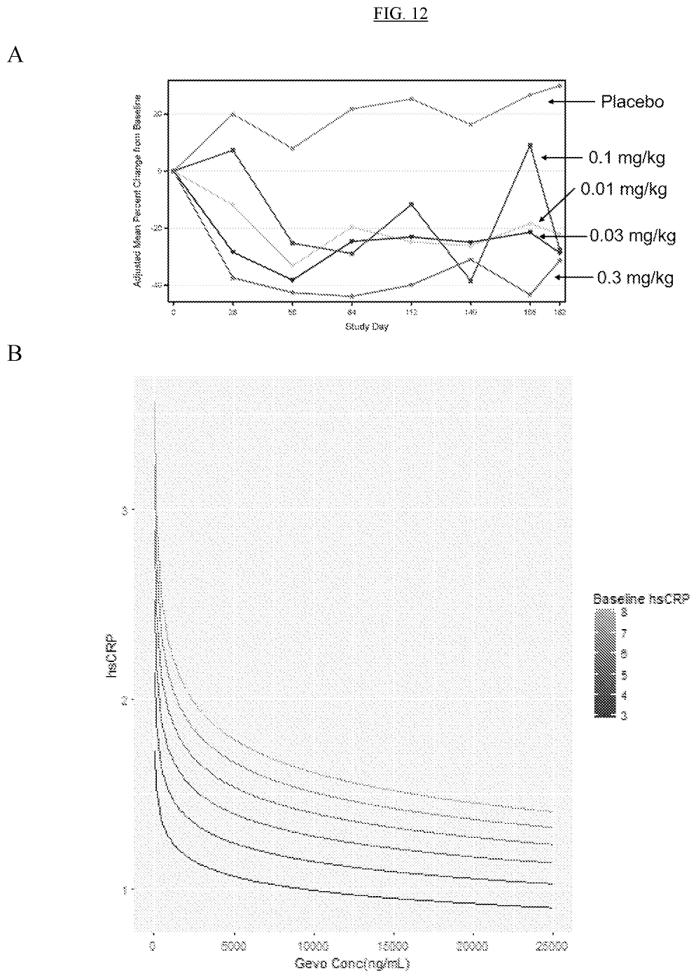

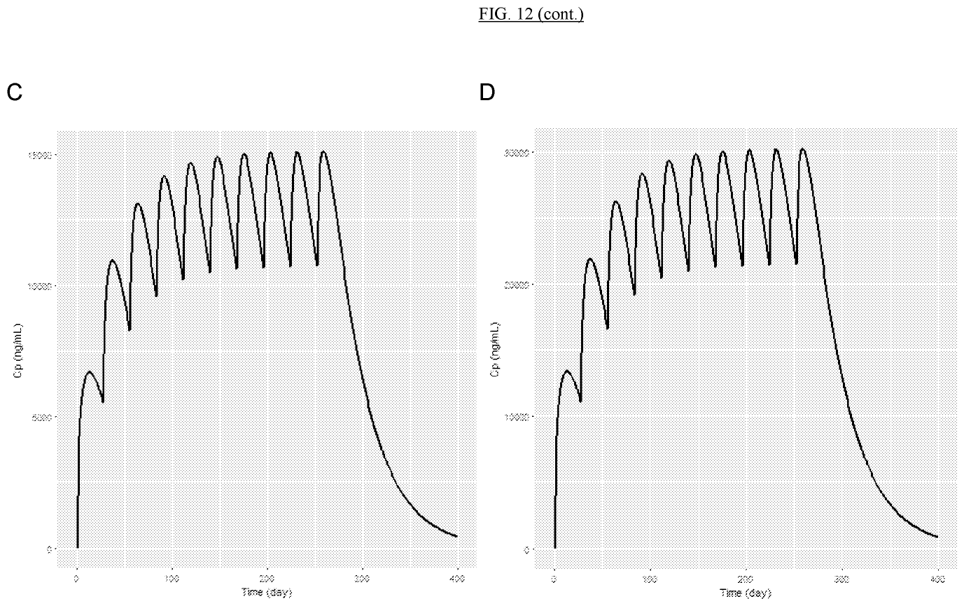

Gevokizumab (IC50 of ˜2-5 μM) shows a ˜10 times higher in virto potency compared to canakinumab (IC50 of ˜42±3.4 μM). The gevokizumab top dose of 0.3 mg/kg (˜20 mg) Q4W showed reduction of hsCRP could reduce hsCRP up to 45% in type 2 diabetes patients (see FIG. 12a).

Next, a pharmacometric model was used to explore the hsCRP exposure-response relationship, and to extrapolate the clinical data to higher ranges. As clinical data show a linear correlation between the hsCRP concentration and the concentration of gevokizumab (both in log-space), a linear model was used. The results are shown in FIG. 12b. Based on that simulation, a gevokizumab concentration between 10000 ng/mL and 25000 ng/mL is optimal because hsCRP is greatly reduced in this range, and there is only a diminishing return with gevokizumab concentrations above 15000 ng/mL. However gevokizumab concentrations between 4000 ng/mL and 10000 ng/mL is expected to be efficacious as hsCRP has already been significantly reduced in that range.

Clinical data showed that gevokizumab pharmacokinetics follow a linear two-compartment model with first order absorption after a subcutaneous administration. Bioavailability of gevokizumab is about 56% when administered subcutaneously. Simulation of multiple-dose gevokizumab (SC) was carried out for 100 mg every four weeks (see FIG. 12c) and 200 mg every four weeks (see FIG. 12d). The simulations showed that the trough concentration of 100 mg gevokizumab given every four weeks is about 10700 ng/mL. The half-life of gevokizumab is about 35 days. The trough concentration of 200 mg gevokizumab given every four weeks is about 21500 ng/mL.

### Example 6

### Preclinical Data on the Effects of Anti-IL-1Beta Treatment.

Canakinumab, an anti-IL-1β human IgG1 antibody, cannot directly be evaluated in mouse models of cancer due to the fact that it does not cross-react with mouse IL-1β. A mouse surrogate anti-IL-1β antibody has been developed and is being used to evaluate the effects of blocking IL-1β in mouse models of cancer. This isotype of the surrogate antibody is IgG2a, which is closely related to human IgG1.

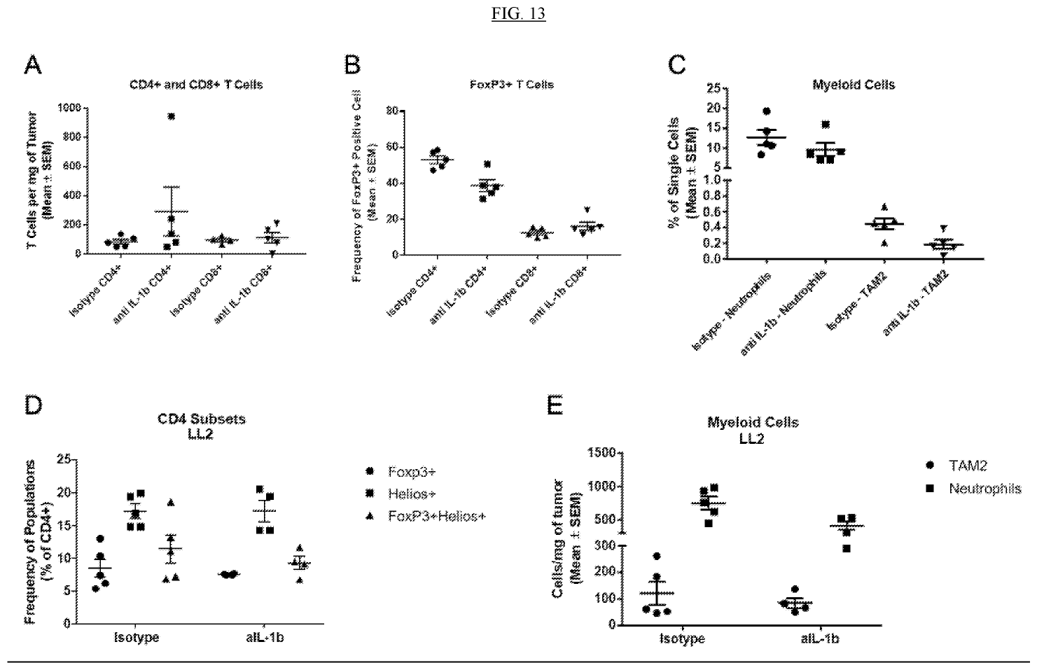

In the MC38 mouse model of colon cancer, modulation of tumor infiltrating lymphocytes (TILs) can be seen after one dose of the anti-IL-1β antibody (FIG. 13a-c). MC38 tumors were subcutaneously implanted in the flank of C57BL/6 mice and when the tumors were between 100-150 mm3, the mice were treated with one dose of either an isotype antibody or the anti IL-1β antibody. Tumors were then harvested five days after the dose and processed to obtain a single cell suspension of immune cells. The cells were then ex vivo stained and analyzed via flow cytometry. Following a single dose of an IL-1β blocking antibody, there is an increase in in CD4+ T cells infiltrating the tumor and also a slight increase in CD8+ T cells (FIG. 13a). The CD8+ T cell increase is slight but may allude to a more active immune response in the tumor microenvironment, which could potentially be enhanced with combination therapies. The CD4+ T cells were further subdivided into FoxP3+ regulatory T cells (Tregs), and this subset decreases following blockade of IL-1β (FIG. 13b). Among the myeloid cell populations, blockade of IL-1β results in a decrease in neutrophils and the M2 subset of macrophages, TAM2 (FIG. 13c). Both neutrophils and M2 macrophages can be suppressive to other immune cells, such as activated T cells (Pillay et al, 2013; Hao et al, 2013; Oishi et al 2016). Taken together, the decrease in Tregs, neutrophils, and M2 macrophages, in the MC38 tumor microenvironment following IL-1β blockade argues that the tumor microenvironment is becoming less immune suppressive.

In the LL2 mouse model of lung cancer, a similar trend towards a less suppressive immune microenvironment can be seen after one dose of an anti-IL-1β antibody (FIG. 13d-f). LL2 tumors were subcutaneously implanted in the flank of C57BL/6 mice and when the tumors were between 100-150 mm3, the mice were treated with one dose of either an isotype antibody or the anti-IL-1β antibody. Tumors were then harvested five days after the dose and processed to obtain a single cell suspension of immune cells. The cells were then ex vivo stained and analyzed via flow cytometry. There is a decrease in the Treg populations as evaluated by the expression of FoxP3 and Helios (FIG. 13d). FoxP3 and Helios are both used as markers of regulatory T cells, while they may define different subsets of Tregs (Thornton et al, 2016). Similar to the MC38 model, there is a decrease in both neutrophils and M2 macrophages (TAM2) following IL-1β blockade (FIG. 13e). In addition to this, in this model the change in the myeloid derived suppressor cell (MDSC) populations were evaluated following antibody treatment. The granulocytic or polymorphonuclear (PMN) MDSC were found in reduced numbers following anti-IL-1β treatment (FIG. 13f). MDSC are a mixed population of cells of myeloid origin that can actively suppress T cell responses through several mechanisms, including arginase production, reactive oxygen species (ROS) and nitric oxide (NO) release (Kumar et al, 2016; Umansky et al, 2016). Again, the decrease in Tregs, neutrophils, M2 macrophages, and PMN MDSC in the LL2 model following IL-1β blockade argues that the tumor microenvironment is becoming less immune suppressive.

TILs in the 4T1 triple negative breast cancer model also show a trend towards a less suppressive immune microenvironment after one dose of the mouse surrogate anti-IL-1β antibody (FIG. 13g-j). 4T1 tumors were subcutaneously implanted in the flank of Balb/c mice, and the mice were treated with either an isotype antibody or the anti-IL-1β antibody when the tumors were between 100-150 mm3. Tumors were then harvested five days after the dose and processed to obtain a single cell suspension of immune cells. The cells were then ex vivo stained and analyzed via flow cytometry. There is a decrease in CD4+ T cells after a single dose of an anti-IL-1β antibody (FIG. 13g) and within the CD4+ T cell population, there is a decrease in the FoxP3+ Tregs (FIG. 13h). Further, there is a decrease in both the TAM2 and neutrophil populations following treatment of the tumor-bearing mice (FIG. 13i). All of these data together again argue that IL-1β blockade in the 4T1 breast cancer mouse model leads to a less suppressive immune microenvironment. In addition to this, in this model the MDSC populations was also evaluated following antibody treatment. Both the granulocytic (PMN) MDSC and monocytic MDSC were found in reduced numbers following anti-IL-1β treatment (FIG. 13j). These findings in combination with the changes in Tregs, M2 macrophages, and the neutrophil populations describe a decrease in the immune suppressive tumor microenvironment in the 4T1 tumor model.

While these data are from colon, lung, and breast cancer models, the data can be extrapolated to other types of cancer. Even though these models do not fully correlate to human cancers of the same type, the MC38 model in particular is a good surrogate model for hypermutated/MSI (microsatellite instable) colorectal cancer (CRC). Based on the transcriptomic characterization of the MC38 cell line, four of the driver mutations in this line correspond to known hotspots in human CRC, although these are at different positions (Efremova et al, 2018). While this does not make the MC38 mouse model identical to human CRC, it does mean that MC38 may be a relevant model for human MSI CRC. Generally, mouse models do not always correlate to the same type of cancer in humans due to genetic differences in the origins of the cancer in mice versus humans. However, when examining the infiltrating immune cells, the type of cancer is not always important, as the immune cells are more relevant. In this case, as three different mouse models show a similar decrease in the suppressive microenvironment of the tumor, blocking IL-1β seems to lead to a less suppressive tumor microenvironment. The extent of the change in immune suppression with multiple cell types (Tregs, TAMs, neutrophils) showing a decrease compared to the isotype control in multiple tumor syngeneic mouse tumor models is a novel finding for IL-1β blockade in mouse models of cancer. While suppressor cell decreases have been seen before, multiple cell types in each model is a novel finding. In addition, changes to MDSC populations in the 4T1 and Lewis lung carcinoma (LL2) models have been seen downstream of IL-1β, but the finding in the LL2 model that blockade of IL-1β can lead to the reduction of MDSCs is novel to this study and the mouse surrogate of canakinumab (Elkabets et al, 2010).

Even though these models do not fully correlate to human cancers of the same type, the MC38 model in particular is a good surrogate model for hypermutated/MSI (microsatellite instable) colorectal cancer (CRC). Based on the transcriptomic characterization of the MC38 cell line, four of the driver mutations in this line correspond to known hotspots in human CRC, although these are at different positions (Efremova et al, 2018). While this does not make the MC38 mouse model identical to human CRC, it does mean that MC38 may be a relevant model for human MSI CRC (Efremova M, et al. Nature Communications 2018; 9: 32)

### Example 7

Preclinical Data on the Efficacy of Canakinumab in Combination with an Anti-PD-1 (Pembrolizumab) in the Treatment of Cancer.

A pilot study was designed to assess the impact of canakinumab as a monotherapy or in combination with anti-PD-1 (pembrolizumab) on tumor growth and the tumor microenvironment. A xenograft model of human NSCLC was created by subcutaneous injection of a human lung cancer cell line H358 (KRAS mutant) into BLT mouse xenograft model.

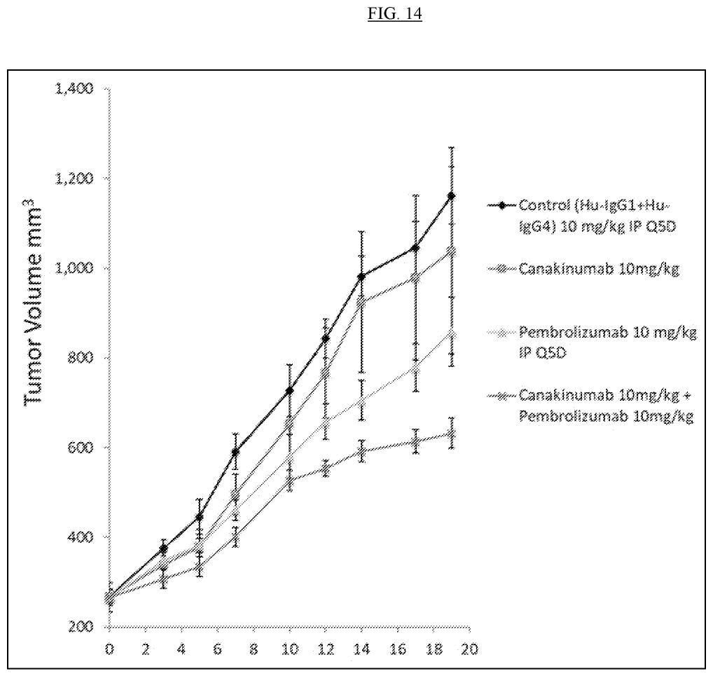

As shown in FIG. 14, the H358 (KRAS mutant) model is a very fast growing and aggressive model. In this model, combination treatment of canakinumab and pembrolizumab (shown in purple) led to a greater reduction than canakinumab single agent arm (shown in red) and pembrolizumab single agent treatment (shown in green), with a 50% decrease observed in the mean tumor volume when compared to the vehicle group.

### Example 8

Preclinical Data on the Efficacy of Canakinumab in Combination with Docetaxel in the Treatment of Cancer.

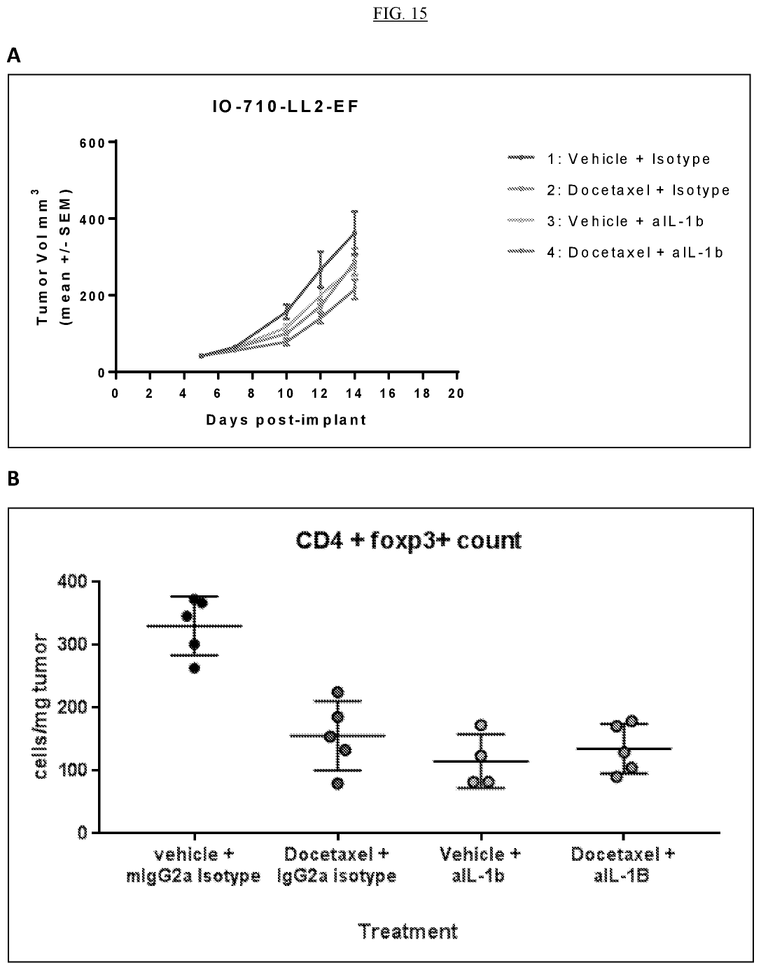

In a study of anti-IL-1β in combination with docetaxel in an aggressive lung model (LL2), modest efficacy with anti-IL-1β was observed, as well as docetaxel alone. The efficacy was enhanced in the combination compared to either group alone or control (FIG. 15A). Decreases in immunosuppressive cells were observed with anti-IL-1β alone or in combination at the PD time point 5 days after the first dose, specifically in regulatory T cells and suppressive mouse myeloid cells including neutrophils, monocytes and MDSCs in tumors after IL-1β inhibition (FIG. 15B-E). These data support that the proposed mechanism of action in IL-1β inhibition can be demonstrated in vivo and also some efficacy of anti-IL-1β monotherapy was observed.

### Example 9

Treatment of 4T1 Tumors with 01BSUR and Docetaxel Leads to Alterations in the Tumor Microenvironment.

Female Balb/c mice with 4T1 tumors implanted subcutaneously (s.c.) on the right flank were treated 8 and 15 days post-tumor implant initiating when the tumors reached about 100 mm³ with the isotype antibody, docetaxel, 01BSUR, or a combination of docetaxel and 01BSUR. 01BSUR is the mouse surrogate antibody, since canakinumab does not cross-react to murine IL-1beta. 01BSUR belongs to the mouse IgG2a subclass, which corresponds to human IgG1 subclass, which canakinumab belongs to. 5 days after the first dose, tumors were harvested and analyzed for changes to the infiltrating immune cell populations. This was done again at the end point of the study, 4 days after the second dose.

### Tumor Burden

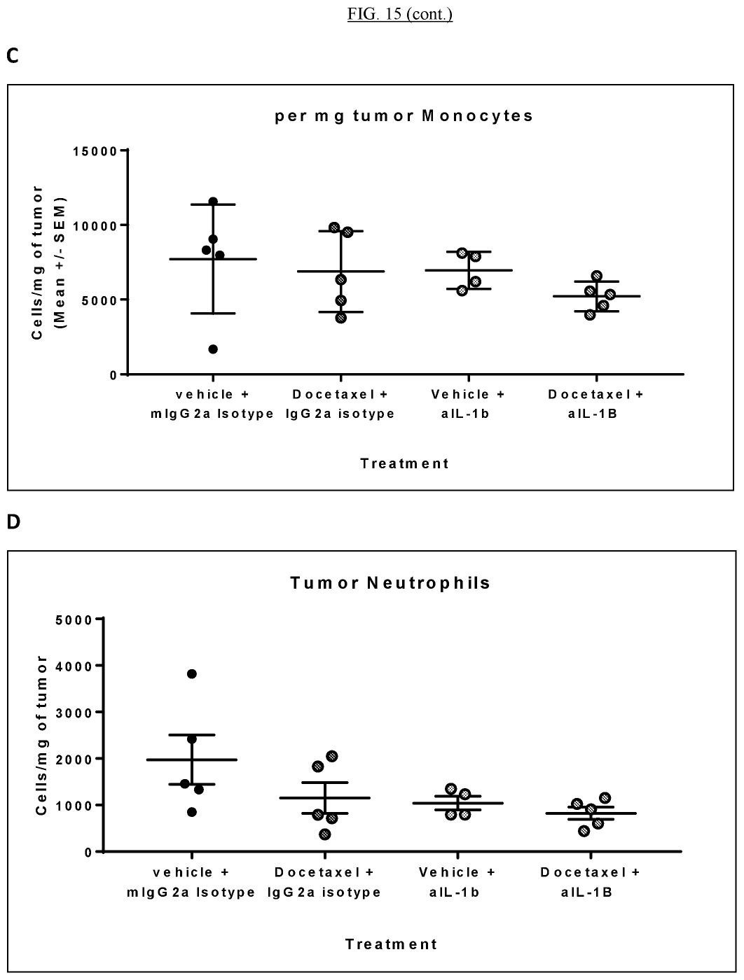

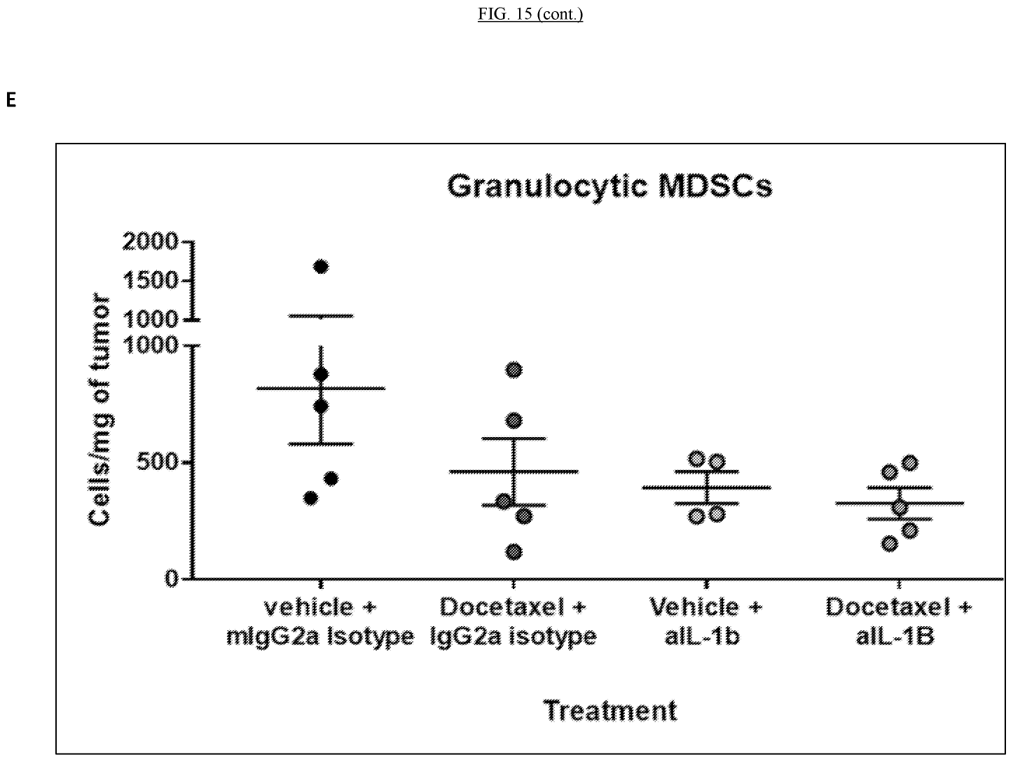

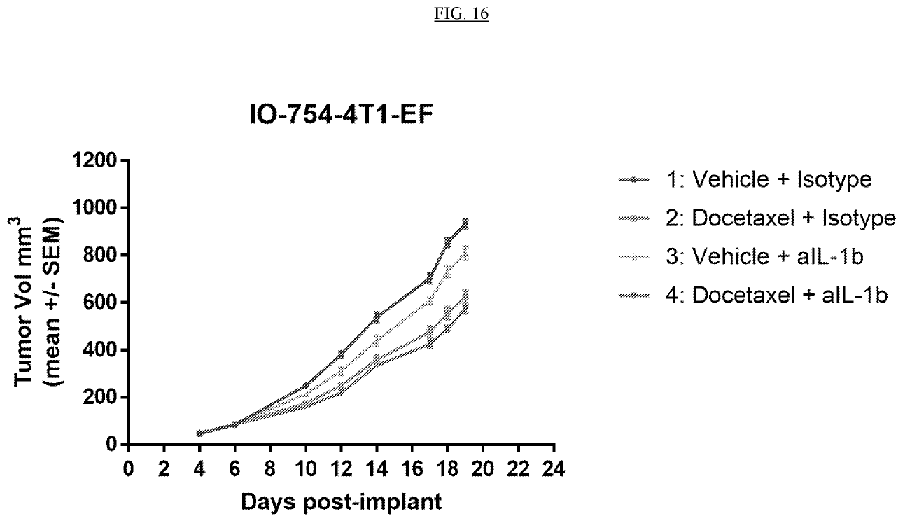

A slight slowing in tumor growth was seen in the 01BSUR anti-IL-1 alone treatment group compared to the vehicle/isotype control. This delay was enhanced in the single agent docetaxel group. The combination group showed a similar slowing in growth as the docetaxel alone group (FIG. 16).

TIL Analysis of 4T1 Tumors after a Single Dose of Docetaxel and 01BSUR—Myeloid Panel

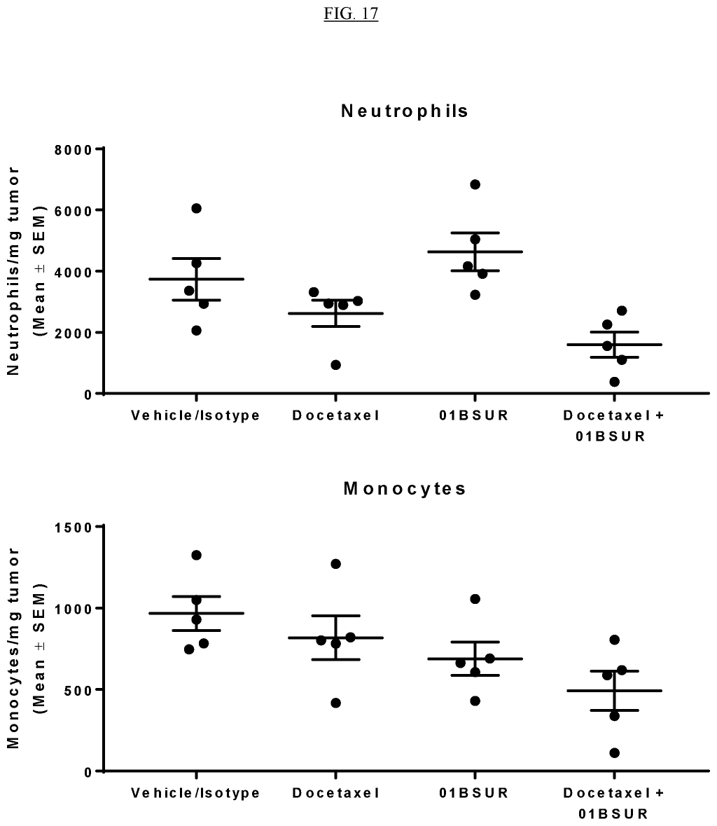

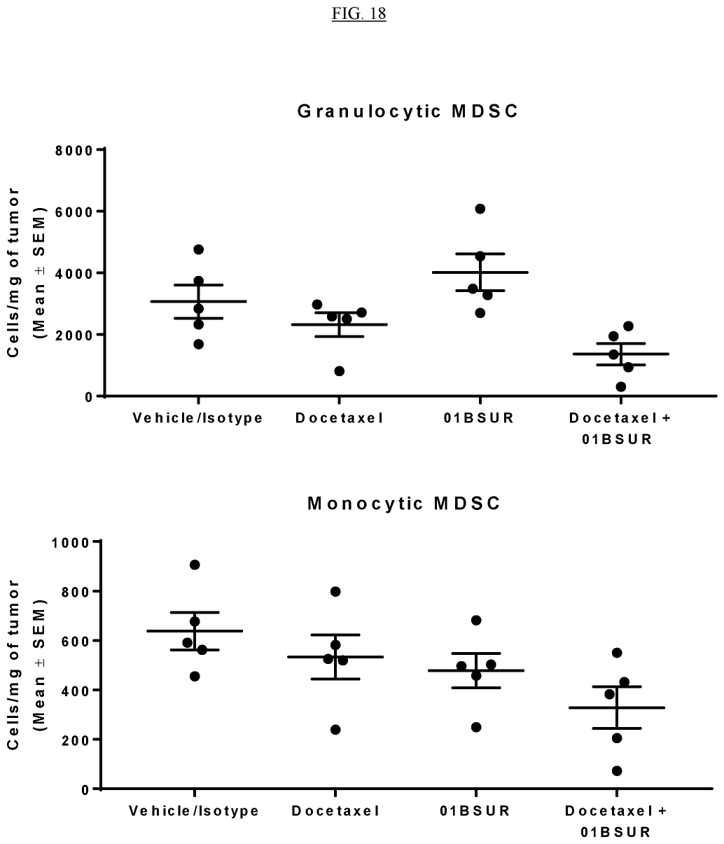

Following a single treatment with docetaxel alone or in combination with 01BSUR, there was a decrease in neutrophils in the 4T1 tumors. The combination group, showed a greater decrease in neutrophil cell number than the docetaxel single agent group. Single agent 01BSUR led to a slight increase in neutrophils in 4T1 tumors, although this was not a significant change compared to the control group. Each of the treatments led to a decrease in monocytes compared to the vehicle/isotype group. The single agent 01BSUR treatment led to a greater decrease in monocytes than the docetaxel alone group. Further, the combination showed an even greater decrease in monocytes compared to the control group (P=0.0481) (FIG. 17). Similar trends to the granulocytes and monocytes were seen among the granulocytic and monocytic Myeloid derived suppressor cells (MDSC). Docetaxel alone and in combination with 01BSUR led to a decrease in granulocytic MDSC. All treatments led to a decrease in monocytic MDSC, with the combination leading to a greater decrease than either of the single agents (FIG. 18).

TIL Analysis of 4T1 Tumors after a Second Dose of Docetaxel and 01BSUR

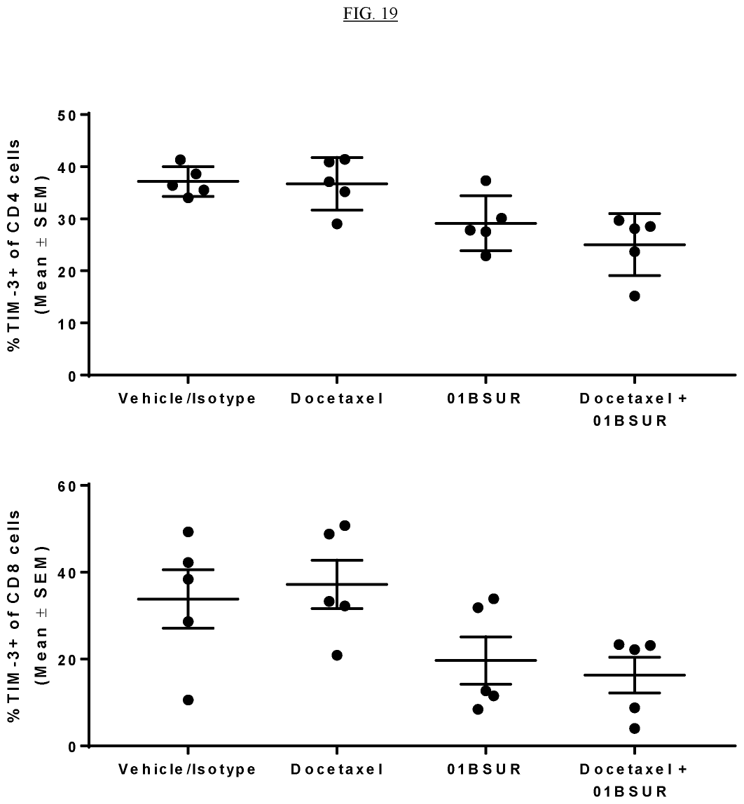

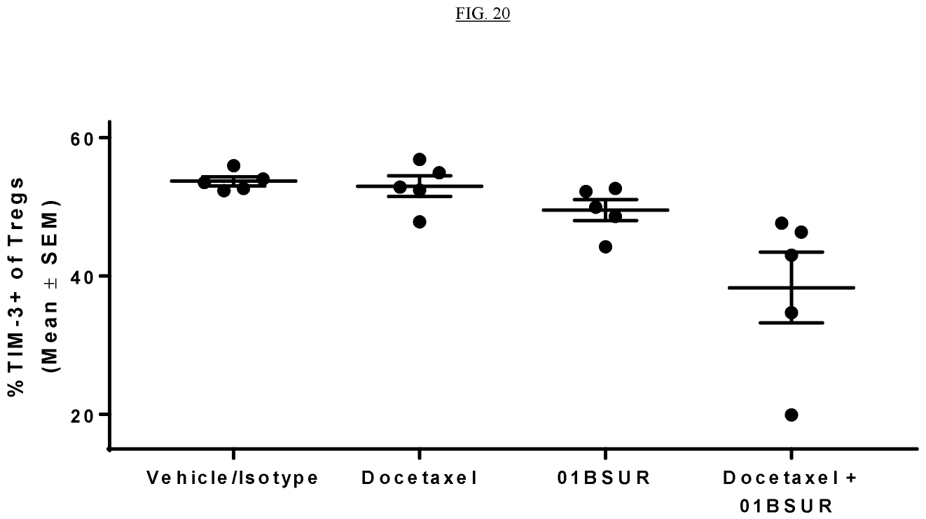

Four days after a second dose of docetaxel and 01BSUR 4T1 tumors were analyzed for immune cell infiltrates. The percent of both CD4⁺ and CD8⁺ T cells expressing TIM-3 were determined. Docetaxel alone led to no change in the TIM-3 expressing cells compared to the control group, while there was a decrease in the TIM-3 expressing cells following treatment with 01BSUR alone or in combination with docetaxel. The combination group, appears to show a slightly larger decrease in TIM-3 expressing cells than the single agent 01BSUR group (P=0.0063) for CD4⁺ T cells compared to control (FIG. 19). Similar trends were seen in the Treg subset of cells with the combination group showing the largest level of decrease of the TIM-3 expressing cells (P=0.0064) compared to the control (FIG. 20).

### Conclusion and Discussion

Blocking IL-1β has been shown to be a potent method of changing the inflammatory microenvironment in autoimmune disease. ACZ885 (canakinumab) has been highly effective at treating some inflammatory autoimmune diseases, such as CAPS (Cryopyrin Associated Periodic Syndrome). As many tumors have an inflammatory microenvironment, blocking IL-1β is being studied to determine the impact that this will have on the tumor microenvironment alone and in combination with agents that will work to block the PD-1/PD-L1 axis or standard of care chemotherapeutic agents such as docetaxel. It has been shown through preclinical experiments and the CANTOS trial that the blockade of IL-1β can have an impact on tumor growth and development. However, the CANTOS trial, an atherosclerosis trial, evaluated this in a prophylactic setting with patients with no known or detectable cancer at the time of enrollment. Patients with established tumors or metastases may have different levels of response to IL-1β blockade.

These preliminary results studying combinations of 01BSUR, a murine surrogate of ACZ885, and docetaxel show that in the LL2 and 4T1 tumors models, this combination can have an impact on tumor growth.

The studies described here examine the TILs following a single treatment only (1D2 and 01BSUR combinations) or following two doses of each treatment (01BSUR and docetaxel). The overall trends alludes to a change in the suppressive nature of the TME in LL2 and 4T1 tumors.

While there is not a consistent change in the overall CD4+ and CD8+ T cells in the TME of these tumors, there is a trend towards in decrease in the Tregs in these tumors. Additionally, the Tregs typically also show a decrease in the percentage of cells expressing TIM-3. Tregs that express TIM-3 may be more effective suppressors of T cells than non-TIM-3 expressing Tregs [Sakuishi, 2013]. In several of the studies, there is an overall decrease of TIM-3 on all T cells. While the impact of this on these cells is not yet known, TIM-3 is a checkpoint and these cells may be more activated than the TIM-3 expressing T cells. However, further work is needed to understand these changes as some of the T cell changes observed could allude to a therapy that is less effective than the control.

While T cells make up a portion of the immune cell infiltrate in these tumors, a large portion of the infiltrating cells are myeloid cells. These cells were also analyzed for changes and IL-1β blockade consistently led to a decrease in the numbers of neutrophils and granulocytic MDSC in the tumors. Often these were accompanied by decreased monocytes and monocytic MDSC; however, there was more variability in these populations. Neutrophils both produce IL-1β and respond to IL-1β while MDSC generation is often dependent on IL-1β, and both subsets of cells can suppress the function of other immune cells. Decreases in both neutrophils and MDSC combined with a decrease in Tregs may mean that the tumor microenvironment becomes less immune suppressive following IL-1β blockade. A less suppressive TME may lead to a better anti-tumor immune response, particularly with checkpoint blockade.

These data taken together show that blocking both IL-1β and the PD-1/PD-L1 axis may lead to a more immune active tumor microenvironment or combining IL-1β blockade with chemotherapy may have a similar impact.

### Example 10

### Determining Immunogenicity/Allergenicity to IL-1β Antibody

During the CANTOS trial, blood samples for immunogenicity assessments were collected at baseline Month 12, 24 and end of study visit. Immunogenicity was analyzed using a bridging immunogenicity electrochemiluminescence immunoassay (ECLIA). Samples were pre-treated with acetic acid and neutralized in buffer containing labeled drug (biotinylated ACZ885 and sulfo-TAG (Ruthenium) labeled ACZ885). Anti-canakinumab antibodies (anti-drug antibodies) were captured by a combination of biotinylated and sulfo-TAG labeled forms of ACZ885. Complex formation was subsequently detected by electrochemiluminescence by capturing complexes on Mesoscale Discovery Streptavidin (MSD) plates.

Treatment-emergent anticanakinumab antibodies (anti-drug antibodies) were detected in low and comparable proportions of patients across all treatment groups (0.3%, 0.4% and 0.5% in the canakinumab 300 mg, 150 mg and placebo groups respectively) and were not associated with immunogenicity related AEs or altered hsCRP response.

### Example 11

Biomarker analysis from the CANTOS trial patients with gastroesophageal cancer, colorectal cancer and pancreatic cancer were grouped into GI group. Patients with bladder cancer, renal cell carcinoma and prostate cancer were grouped into GU group. Within the group, patients were further divided according to their baseline IL-6 or CRP level into above median group and below median group. The mean and median of time to cancer event were calculated as shown the table below.

There seems to have a trend that patient group have below median level of CRP and IL-6 had in general longer time to develop cancer. This trend seems to be stronger based on IL-6 analysis than CRP, possibly due to the fact that IL-6 is immediately downstream of IL-1b, where CTP is further away from IL-1b signaling and therefore could be influenced by other factors as well.

TABLE 12

| | | Time to cancer AE | | |
|---|---|---|---|---|
| Set | IL-6 median | N | Mean | Median |
| GI | Above median | 34 | 18.35 | 16.03 |
| | Below median | 35 | 27.84 | 28.55 |
| GU | Above median | 33 | 21.79 | 17.45 |
| | Below median | 33 | 27.85 | 23.39 |

TABLE 13

| | | Time to cancer AE | | |
|---|---|---|---|---|
| | CRP Median | N | Mean | Median |
| GI | Above Median | 56 | 19.23 | 15.08 |
| | Below Median | 58 | 25.61 | 26.17 |
| GU | Above Median | 54 | 24.57 | 23.23 |
| | Below Median | 56 | 25.15 | 24.13 |

### Example 12

| | |
|---|---|
| Full Title | A Phase Ib, multicenter, open-label study of MBG453 and |
| | canakinumab in adult patients with lower risk myelodysplastic syndrome |
| Purpose and | The purpose of this study is to identify a recommended dose of |
| rationale | MBG453 in combination with canakinumab in patients with lower |
| | risk MDS with anemia, thrombocytopenia or neutropenia that are |
| | considered to require treatment by the treating physician and for |
| | which there are no standard of care treatment options. The safety, |
| | tolerability, pharmacokinetics, pharmacodynamics and preliminary |
| | activity of MBG453 in combination with canakinumab will also be assessed. |
| | MBG453 |
| | TIM-3 is expressed on the majority of CD34+CD38− leukemic stem |
| | cells (LSCs) and CD34+CD38+ leukemic progenitors in AML and |
| | MDS, not but in CD34+CD38 normal hematopoeitic stem cells |
| | (HSCs) (Kikushige et al. 2010; Jan et al. 2011). Functional evidence |
| | for a key role for TIM-3 in AML was established by use of an anti- |
| | TIM-3 antibody which inhibited engraftment and development of |
| | human AML in immunodeficient murine hosts (Kikushige et al. |
| | 2010). Upregulation of TIM-3 is also associated with leukemic |
| | transformation of pre-leukemic disease, include myelodysplastic |
| | syndromes (MDSs) and myeloproliferative neoplasms (MPNs), |
| | such as chronic myelogenous leukemia (CML) (Kikushige et al. |
| | 2015). TIM-3 expression on MDS blasts was also found to correlate |
| | with disease progression (Asayama et al. 2017). |
| | In addition to its cell-autonomous role on pre-leukemic and |
| | leukemic stem cells, TIM-3 has a widespread and complex role in |
| | immune system regulation, with published roles in both the adaptive |
| | immune response (CD4+ and CD8+ T effector cells, regulatory T |
| | cells) and innate immune responses (macrophages, dendritic cells, |
| | NK cells). TIM-3 has a critical role in tumor-induced immune |
| | suppression as it marks the most suppressed or dysfunctional |
| | populations of CD8+ T cells in animal models of solid and |
| | hematologic malignancies (Sakuishi et al. 2010, Zhou et al. 2011, |
| | Yang et al. 2012) and is expressed on FoxP3+ regulatory T cells |
| | (Tregs), which correlate with disease severity in many cancer |
| | indications (Gao et al. 2012, Yan et al. 2013). TIM-3 is expressed on |
| | exhausted or dysfunctional T cells in cancer, and ex vivo TIM-3 |
| | blockade of TIM-3+ NY-ESO-1+ T cells from melanoma patients |
| | restored IFN-γ and TNF-α production as well as proliferation in |
| | response to antigenic stimulation (Fourcade et al. 2010). Blockade |
| | of TIM-3 on macrophages and antigen cross-presenting dendritic |
| | cells enhances activation and inflammatory cytokine/chemokine |
| | production (Zhang 2011, Zhang 2012, Chiba 2012, de Mingo Pulido |
| | 2018), ultimately leading to enhanced effector T cells responses. |
| | MBG453 is a high-affinity, ligand blocking, humanized anti TIM-3 |
| | IgG4 antibody which blocks the binding of TIM-3 to |
| | phosphatidylserine (PtdSer). Given the important |
| | immunomodulatory role of TIM-3 in both innate and adaptive |
| | immunity, as well as its expression on leukemic stem cells in AML |
| | and MDS, an anti-TIM-3 antibody may not only help to restore an |
| | anti-tumor immune response, but may additionally directly target |
| | MDS stem cells. As a result, MBG453 may have both direct and |
| | indirect disease-modifying activity in low-risk MDS which could |
| | be augmented by therapies directed at pro-inflammatory pathways, |
| | including IL-1β blockade. |
| | Canakinumab |
| | IL-1β is a secreted pleotropic cytokine with a central role in |
| | inflammation and immune response. Increases in IL-1β are |
| | observed in multiple clinical settings including cancer (Apte et al. |
| | 2006); (Dinarello 2010). Secreted IL-1β, derived from the tumor |
| | microenvironment and by malignant cells, promotes tumor cell |
| | proliferation, increases invasiveness and dampens anti-tumor |
| | immune response, in part by recruiting inhibitory neutrophils (Apte |
| | et al. 2006; Miller et al. 2007). Experimentally, inhibition of IL-1β |
| | results in a decrease in tumor burden and metastasis (Voronov et al. 2003). |
| | Multiple lines of scientific investigation suggest the elevated levels |
| | of IL-1β may be playing a causative role in the pathogenesis of |
| | MDS. Importantly, polymorphisms in the IL-1β gene confer a |
| | significantly increased susceptibility to MDS (Yin et al. 2016). In |
| | studies of bone marrow aspirates from MDS patients, frequent |
| | elevations in IL-1β and downregulation of the antagonist IL-1ra |
| | were demonstrated, suggesting a state of unbalanced IL-1β |
| | activation (Preisler et al. 1999). Elevated levels of IL-1β are capable |
| | of blocking erythropoietin's proliferative effects on erythroid |
| | progenitor cells in vitro (Schooley et al. 1987) and chronic exposure |
| | of hematopoietic stem cells to elevated IL-1β promoted myeloid |
| | differentiation, suppressed erythroid differentiation and led to |
| | hematopoietic stem cell exhaustion in vivo (Pietras et al. 2016). In |
| | the context of cancer, IL-1β derived from tumor cells and/or the |
| | microenvironment can activate local inflammation and potentiate |
| | carcinogenesis (Apte et al. 2006), perhaps by impairing anti-cancer |
| | immunity through stimulation of MDSCs (Ganan-Gomez et al. 2015). |
| | Canakinumab is a high-affinity human monoclonal anti-human |
| | interlukin-1β, which binds IL-1β and inhibits IL-1-mediated |
| | signaling. Given the central role of IL-1β and the inflammasome in |
| | the pathogenesis of MDS, canakinumab may target the underlying |
| | pro-inflammatory signaling driving the myelodysplastic syndrome |
| | phenotype, and could cooperate with therapies directed against |
| | MDS stem cells, such as MBG453. |
| Primary | The primary objective of this study is to characterize the safety, |
| Objective(s) | tolerability and identify the recommended dose for MBG453 in |
| | combination with canakinumab in patients with lower risk MDS |
| | who have anemia, thrombocytopenia or neutropenia that are |
| | considered to require treatment by the treating physician and for |
| | which there are no standard of care treatment options. |
| | Incidence of dose limiting toxicities (DLTs) |
| | Incidence and severity of AEs and SAEs, including |
| | changes in laboratory values, vital signs and ECGs |
| | Dose interruptions, reductions and dose intensity |
| Secondary | To evaluate the preliminary efficacy and duration of effect |
| Objectives | of MBG453 in combination with canakinumab |
| | To evaluate the pharmacokinetic (PK) profile of each |
| | combination partner |
| | To characterize the prevalence and incidence of |
| | immunogenicity of each combination partner |
| Study design | Evaluation of MBG453 in combination with canakinumab |
| Population | This study will be conducted in adult patients with a confirmed |
| | diagnosis of IPSS-R-defined very low, low or intermediate-risk |
| | myelodysplastic syndrome (MDS) with one or more of the |
| | following: |
| | Anemia that is relapsed, refractory or intolerant to ESAs, and |
| | considered to require treatment by the treating physician |
| | Anemia that is ESA-naive with EPO level ≥500 mU/mL and |
| | considered to require treatment by the treating physician |
| | Thrombocytopenia amenable to response assessments by IWG |
| | and considered to require treatment by the treating physician |
| | Neutropenia amenable to response assessments by IWG and which |
| | is relapsed, refractory or intolerant to growth factors and considered |
| | to require treatment by the treating physician |
| Key Inclusion | 1. Subjects must have a diagnosis of International Prognostic |
| criteria | Scoring System Revised (IPSS-R) very low, low, or intermediate |
| | risk myelodysplastic syndromes (MDS) with one or more of the |
| | following: |
| | a. Anemia that is relapsed, refractory or intolerant to ESAs and |
| | considered to require treatment by the treating physician |
| | b. Anemia that is ESA-naive with EPO level ≥500/uL and |
| | considered to require treatment by the treating physician |
| | c. Thrombocytopenia amenable to response assessments by IWG |
| | and considered to require treatment by the treating physician |
| | d. Neutropenia with absolute neutrophil count (ANC) ≥500/uL but |
| | amenable to response assessments by IWG and which is relapsed, |
| | refractory or intolerant to growth factors and considered to require |
| | treatment by the treating physician t |
| | 2. Eastern Cooperative Oncology Group (ECOG) performance |
| | status (PS) ≤2 |
| | 3. Patient must be a candidate for serial bone marrow aspirate and/or |
| | biopsy according to the institutions guidelines and be willing to |
| | undergo a bone marrow aspirate and/or biopsy at screening, during |
| | and at the end of therapy on this study. |
| Key Exclusion | 1. Systemic antineoplastic therapy (including cytotoxic |
| criteria | chemotherapy, alpha-interferon, kinase inhibitors or other |
| | targeted small molecules, and toxin-immunoconjugates) or any |
| | experimental therapy within 14 days or 5 half-lives, whichever |
| | is shorter, before the first dose of study treatment. |
| | 2. History of hypersensitivity to any of the study treatments or its |
| | excipients or to drugs of similar chemical classes. |
| | 3. Patients with ≥10% bone marrow blasts |
| | 4. Patients with an Absolute Neutrophil Count (ANC) <0.5/μL |
| | 5. Patients with Chronic Myelomonocytic Leukemia (CMML) or |
| | Myelodysplastic/myeloproliferative neoplasms (MDS/MPN) |
| | 6. Use of hematopoietic colony-stimulating growth factors (e.g. G- |
| | CSF, GM-CSF, M-CSF), thrombopoietin mimetics or erythroid |
| | stimulating agents anytime ≤2 weeks (or 5 half-lives, whichever is |
| | longer) prior to start of study treatment. |
| | 7. Systemic chronic corticosteroid therapy (>10 mg/day prednisone |
| | or equivalent) or any immunosuppressive therapy within 7 days of |
| | first dose of study treatment. Topical, inhaled, nasal and ophthalmic |
| | steroids are allowed. |
| Study treatment | MBG453, canakinumab |
| Efficacy assessments | Reduction in red blood cell/platelet transfusions from baseline |
| | Development of transfusion independence for ≥8 weeks or longer |
| | Hb, Plt, ANC/WBC change from baseline |
| | BOR per IWG (CR, Cytogenetic Response, HI [HI-E/HI-P/HI- |
| | N (≥8 weeks)], SD), as well as CRh |
| | Time to onset of transfusion independence/BOR, and duration of |
| | response |
| Pharmacokinetic | Serum concentrations of each combination partner and derived PK |
| assessments | parameters |
| | Antidrug antibodies (ADA) prevalence at baseline and on |
| | treatment |
| Other assessments | To evaluate predictors of efficacy/response, including determining |
| | whether genetic mutations or changes in biomarkers predict the |
| | degree or duration of efficacy responses |
| | To characterize the effect of study treatment on immune cell |
| | subsets in the bone marrow using flow cytometry |
| | To characterize treatment-related modulation of gene expression |
| | in both malignant and immune cells using scRNA-seq |
| | To evaluate the effect of treatment on circulating CHIP burden |
| | To assess the relationship between systemic exposure (PK) and |
| | response measures of clinical efficacy, toxicity, and |
| | pharmacodynamics (including soluble markers such as TIM-3, |
| | TGFβ1, hsCRP, IL-1β). |

### Example 13

Canakinumab in Incident Anemia—CANTOS Trial

Of 10,061 participants randomized, baseline anemia (hemoglobin less than 12 g/dL for women and 13 g/dL for men) was present in 417 women and 899 men, while 4 participants did not have baseline hemoglobin measurements. Thus, 8,741 CANTOS participants were included in this analysis. Blood samples were obtained from all trial participants in the canakinumab and placebo groups at randomization and during the trial (baseline, 3, 6, 9, 12, 18, 24, 30, 36, 42, 48, 54, and 60 months after randomization). All samples underwent standard hematology assessments, including hemoglobin, hematocrit, red blood cell count, white blood cell count with differential, and platelet count. Follow-up CBCs permitted assessment of incident anemia, defined prospectively as a hemoglobin <13 g/dl for men and <12 g/dl for women arising in individuals with normal hemoglobin upon enrollment in the trial.

The distributions of baseline clinical characteristics that could contribute to anemia (such as age, renal function, hsCRP, alcohol use, diabetes, and hypertension) were compared between placebo or active treatment groups using Chi-square analysis for categorical variables. For continuous variables, Kruskal-Wallis testing was performed for multiple-group comparisons and Wilcoxon rank-sum testing for two group comparisons between placebo and the active treatment group. On a per-protocol pre-specified basis, univariate and adjusted Cox proportional-hazards models stratified by time since index myocardial infarction and trial part were used to estimate relative hazards for incident anemia in the three canakinumab groups (50 mg, 150 mg and 300 mg), compared with those allocated placebo. P-values for the test of trend were calculated across these groups. Scores of 0, 1, 3 and 6 which were proportional to canakinumab doses were used in the trend analysis. Kaplan-Meier curves were constructed to visually evaluate any differences between groups. To parallel treatment response analyses that were pre-specified in the CANTOS protocol for the trial primary cardiovascular endpoints, similar analyses were performed to address whether the magnitude of anti-inflammatory response achieved by individual participants after a single dose of either placebo or canakinumab related to incident anemia. This analysis divided the canakinumab treated participants into two groups according to whether the hsCRP levels fell below 2 mg/L at three months (robust responders) or above 2 mg/L at three months (less robust responders). This time point corresponds with the trough following the first dose, just before the second dose of canakinumab. Additional subgroup analyses were performed assessing factors associated with anemia and chronic inflammation, including age and kidney function. All analyses were by intention to treat. All p-values are two-sided and all confidence intervals calculated at the 95% level.

The 8,741 CANTOS participants without anemia at baseline randomly received placebo, or canakinumab at 50 mg, 150 mg, or 300 mg administered subcutaneously every 3 months. The groups had well matched baseline clinical characteristics, including those predisposing to anemia, such as age, kidney function, and underlying inflammation as assessed by baseline hsCRP (Table 1). Compared to those without anemia at baseline, those with anemia (who were excluded from this secondary analysis) were significantly older, more likely to be female, and had a higher burden of co-morbid illness (higher rates of hypertension and type 2 diabetes mellitus), decreased GFR, and higher levels of hsCRP (Table 1).

TABLE 1
Baseline clinical characteristics of trial participants without
baseline anemia and those excluded for baseline anemia

| | No anemia at | Anemia at |
|---|---|---|
| | baseline | baseline |
| | (n = 8741) | (n = 1316) |
| Age, years | 61.0 (54.0-67.0) | 65.0 (57.0-73.0)* |
| Female sex | 2168 (24.8) | 417 (31.7)* |
| Hemoglobin (g/dL) | | |
| Body-mass index (kg/m²) | 29.9 (26.7-33.8) | 29.0 (25.4-33.7)* |
| Alcohol use (>one drink | 364 (4.17) | 32 (2.43)* |
| per day) | | |
| Hypertension | 6901 (79.0) | 1104 (83.9)* |
| Type 2 Diabetes mellitus | 3351 (38.3) | 676 (51.4)* |
| hsCRP (mg/L) | 4.05 (2.75-6.60) | 5.68 (3.25-10.7)* |
| Estimated GFR (mL/min | 79.0 (66.0-94.0) | 70.0 (53.0-87.0)* |
| per 1.73 m²) | | |

Continuous data are reported as median (IQR), dichotomous data are reported as n(%). Significant between-group differences at baseline are noted. hsCRP = high-sensitivity C-reactive protein. GFR = glomerular filtration rate.
*P < 0.05 for the comparison of participants with anemia at baseline versus those without anemia at baseline.

Baseline levels of hsCRP associated with incident anemia. Specifically, among those with hsCRP levels in the lowest (<3.1 mg/L), middle, and highest (>5.45 mg/L) tertiles, incidence rates of anemia were 5.63, 6.55, and 7.91 per 100 person-years, respectively (P-trend across tertiles <0.0001).

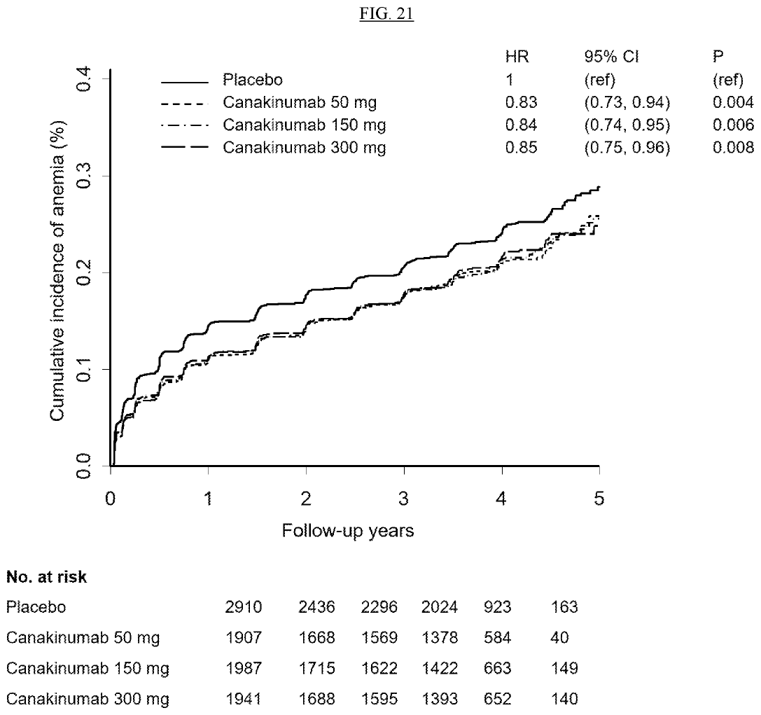

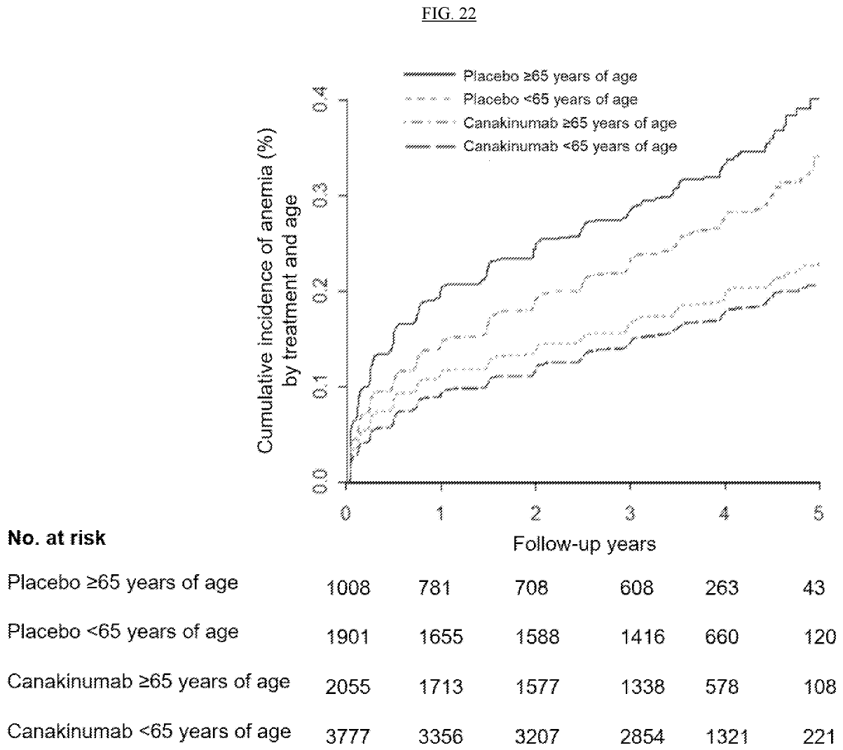

Compared to placebo, participants allocated to any dose of canakinumab had a statistically significant reduction in incident anemia (hemoglobin <13 g/dL in men, <12 g/dL in women) throughout (FIG. 21) as compared to placebo (HR=0.84, 95% CI 0.77-0.93, p<0.0001). The reduction was irrespective of dose: For the 50 mg group (N=1907) the hazard ratio compared to placebo for incident anemia was 0.83 (95% CI 0.73-0.94, P=0.004). For the 150 mg group (N=1987) the hazard ratio compared to placebo for incident anemia was 0.84 (95% CI 0.74-0.95, P=0.006). For the 300 mg group (N=1941) the hazard ratio compared to placebo for incident anemia was 0.85 (95% CI 0.75-0.96, P=0.008). The incidence rates for anemia per 100 person-years was 7.49 in the placebo group, 6.17 in the 50 mg group, 6.33 in the 150 mg group, 6.34 in the 300 mg group and 6.28 in all active doses of canakinumab (p=0.014 for trend across active dose groups compared to placebo). Analysis of combined canakinumab doses compared with placebo demonstrated a pronounced decrease in rates of incident anemia in those patients who achieved on-treatment hsCRP levels less than 2 mg/L after the first dose of canakinumab (HR=0.78, 95% CI 0.70-0.87, p<0.0001). In contrast, individuals with an on-treatment hsCRP ≥2 mg/L, had similar rates of incident anemia as the placebo group (HR 1.01, 95% CI 0.91-1.13, p=0.82). Specifically, among those with hsCRP <2 mg/L three months after initiating canakinumab, HRs for anemia, compared to placebo were 0.67 (95% CI 0.56-0.81, p<0.0001) for the 50 mg group, 0.78 (95% CI 0.67-0.91, p=0.002) for the 150 mg group, 0.76 (95% CI 0.65-0.88, p<0.0001) for the 300 mg group. In contrast, participants who had hsCRP ≥2 mg/L at three months had HRs for anemia, compared to placebo of 0.97 (95% CI 0.84-1.13, p=0.699) for the 50 mg group, 0.89 (95% CI 0.76-1.05, p=0.171) for the 150 mg group, and 1.05 (95% CI 0.88-1.25, p=0.5′70) for the 300 mg group. Notably, the effects of canakinumab on reduction in incident anemia was greater in patients older than 65 years of age (HR=0.78, 95% CI 0.68-0.89, p<0.0001) than patients younger than 65 years of age (HIR=0.88, 95% CI 0.78-1.00, p=0.056) (FIG. 22). Specifically, among participants 65 years or older, the HRs for anemia associated with canakinumab as compared to placebo were 0.80 (9500 CI 0.66-0.96, p=0.01′7) for the 50 mg group, 0.73 (95% CI 0.61-0.88, p=0.001) for the 150 mg group, and 0.80 (95% CI 0.67-0.96, p=0.018) for the 300 mg group (Table 2).

TABLE 2
Risk of incident anemia stratified by high-sensitivity CRP at three months.^{a}

| | | Canakinumab | | | | |
|---|---|---|---|---|---|---|
| | | | | | | p-value |
| | | | | | | (for trend |
| hsCRP < | Placebo | 50 mg | 150 mg | 300 mg | All doses | across |
| 2 mg/L | (n = 2772) | (n = 804) | (n = 1056) | (n = 1225) | (n = 3085) | doses) |
| Incident | 7.59 (669) | 5.01 (136) | 5.94 (211) | 5.69 (235) | 5.60 (582) | 0.000 |
| rate (n) | | | | | | |
| Hazard | 1 (ref) | 0.67 | 0.78 | 0.76 | 0.74 | — |
| Ratio | | (0.56-0.81) | (0.67-0.91) | (0.65-0.88) | (0.67-0.83) | |
| (95% | | | | | | |
| CI) | | | | | | |
| p-value | Ref | 0.000 | 0.002 | 0.000 | 0.000 | — |
| hsCRP | | 50 mg | 150 mg | 300 mg | All doses | |
| 22 mg/L | | (n = 1012) | (n = 827) | (n = 612) | (n = 2445) | |
| Incident | 7.59 (669) | 7.33 (235) | 6.78 (183) | 8.05 (157) | 7.32 (575) | 0.939 |
| rate (n) | | | | | | |
| Hazard | 1 (ref) | 0.97 | 0.89 | 1.05 | 0.97 | — |
| Ratio | | (0.84-1.13) | (0.76-1.05) | (0.88-1.25) | (0.87-1.08) | |
| (95% | | | | | | |
| CI) | | | | | | |
| p-value | Ref | 0.699 | 0.171 | 0.570 | 0.575 | — |

^{a}Incidence rates are per 100 person-years (with numbers of participants with event), p-values for trend and p-values for the combination of all doses are compared to placebo.
CI represents confidence interval,
hsCRP represents high-sensitivity
C-reactive protein.

As also anticipated, participants with an eGFR less than 60 ml/min per 1.73 m² had a higher incidence of anemia compared to participants with an eGFR greater than or equal to 60 mL/min per 1.73 m². Incidence rates for anemia were 14.55 and 11.24 per 100 person-years for placebo and all doses of canakinumab, respectively in participants with an eGFR less than 60 mL/min per 1.73 m² and 6.43 and 5.47 per 100 person-years for placebo and all doses of canakinumab for participants with an eGFR greater than or equal to 60 mL/min per 1.73 m² (Table 3) For participants with an eGFR less than 60 mL/min per 1.73 m² and for those with an eGFR greater than or equal to 60 mL/min per 1.73 m², canakinumab treatment associated with a significant decrease in incident anemia comparing all doses of canakinumab to placebo. HRs for anemia for all groups of canakinumab, compared to placebo, were 0.78 (95% CI 0.65-0.94, p=0.009) for participants with an eGFR less than 60 mL/min per 1.73 m² and 0.85 (95% CI 0.77-0.95, p=0.005) (Table 3).

TABLE 3
Risk of incident anemia stratified by baseline eGFR^{a}

| | | Canakinumab | | | | |
|---|---|---|---|---|---|---|
| | | | | | | value |
| | | | | | | (for |
| | | | | | | trend |
| | Placebo | 50 mg | 150 mg | 300 mg | All doses | across |
| | (n = 3344) | (n = 2170) | (n = 2284) | (n = 2263) | (n = 6717) | doses) |
| eGFR < 60 | | | | | | |
| (mL/min per | | | | | | |
| 1.73 m²) | | | | | | |
| Incident | 14.55 | 11.59 (100) | 10.26 (93) | 11.87 (110) | 11.24 (303) | 0.103 |
| rate (n) | (176) | | | | | |
| Hazard | 1 (ref) | 0.83 | 0.71 | 0.82 | 0.78 | — |
| Ratio (95% | | (0.65-1.07) | (0.55-0.91) | (0.64-1.04) | (0.65-0.94) | |
| CI) | | | | | | |
| p-value | Ref | 0.151 | 0.008 | 0.101 | 0.009 | — |
| eGFR ≥ 60 | | | | | | |
| (mL/min per | | | | | | |
| 1.73 m²) | | | | | | |
| Incident | 6.43 | 5.29 (280) | 5.69 (319) | 5.41 (296) | 5.47 (895) | 0.033 |
| rate (n) | (514) | | | | | |
| Hazard | 1 (ref) | 0.83 | 0.88 | 0.84 | 0.85 | — |
| Ratio (95% | | (0.71-0.96) | (0.76-1.01) | (0.73-0.97) | (0.77-0.95) | |
| CI) | | | | | | |
| p-value | Ref | 0.012 | 0.072 | 0.018 | 0.005 | — |

^{a}Incidence rates are per 100 person-years (with numbers of participants with event), p-values for trend and p-values for the combination of all doses are compared to placebo.
CI represents confidence interval.

These data have practical implications for the use of an IL-1β-binding antibody such as canakinumab as an adjunctive therapy for the treatment of anemia, such as anemia of inflammation.

1-53. (canceled)

54. A method of treating or preventing one or more myelodysplastic syndromes (MDS) in a subject in need thereof comprising administering to the subject a therapeutically effective amount of an IL-1β binding antibody, or functional fragment thereof.

55. The method of claim 54, wherein the one or more MDS have at least a partial inflammatory basis.

56. The method of claim 54, wherein the IL-1β binding antibody, or functional fragment thereof, is canakinumab, or a functional fragment thereof, and wherein canakinumab, or a functional fragment thereof, is administered at a dose of about 150 mg to about 300 mg per treatment, about 200 mg per treatment, or about 250 mg per treatment.

57. The method of claim 54, wherein the subject has high sensitivity C-reactive protein (hsCRP) equal to or greater than about 2 mg/L before first administration of the IL-1β binding antibody or functional fragment thereof.

58. The method of claim 54, wherein the subject has hsCRP equal to or greater than about 4 mg/L before first administration of the IL-1β binding antibody or functional fragment thereof.

59. The method of claim 54, wherein the subject has hsCRP equal to or greater than about 10 mg/L before first administration of the IL-1β binding antibody or functional fragment thereof.

60. The method of claim 54, wherein one or more of the following is true when assessed at least about 3 months after first administration of the IL-1β binding antibody or functional fragment thereof:
(a) the high sensitivity C-reactive protein (hsCRP) level of the subject has been reduced to below about 5 mg/L, about 3.5 mg/L, about 2.3 mg/L, about 2 mg/L, or about 1.8 mg/L; and/or
(b) the high sensitivity C-reactive protein (hsCRP) level of the subject has been reduced by at least about 20% compared to baseline; and/or
(c) the interleukin-6 (IL-6) level of the subject has been reduced by at least about 20% compared to baseline.

61. The method of claim 54, wherein the method comprises administering the IL-1β binding antibody, or functional fragment thereof, about every three weeks or about every four weeks (monthly).

62. The method of claim 54, wherein the IL-1β binding antibody is canakinumab, or a functional fragment thereof.

63. The method of claim 62, wherein the method comprises administering a dose of canakinumab, or a functional fragment thereof, subcutaneously about every three or about every four weeks, wherein the dose is about 200 mg or 250 mg.

64. The method of claim 54, wherein the IL-1β binding antibody is gevokizumab, or a functional fragment thereof.

65. The method of claim 54, wherein the IL-1β binding antibody, or functional fragment thereof, is administered as the first, second, or third line treatment of the one or more MDS.

66. The method of claim 54, wherein the IL-1β binding antibody, or a functional fragment thereof, is administered in combination, as the first, second, or third line treatment of the one or more MDS.

67. A method of treating one or more MDS in a subject in need thereof comprising administering to the subject a therapeutically effective amount of an IL-1β binding antibody, or a functional fragment thereof, in combination with a therapeutically effective amount of a TIM-3 binding antibody, or a functional fragment thereof.

68. The method of claim 67, wherein the one or more MDS are low risk MDS.

69. The method of claim 67, wherein the IL-1β binding antibody, or functional fragment thereof, is canakinumab, or a functional fragment thereof, or gevokizumab, or a functional fragment thereof.

70. The method of claim 67, wherein the TIM-3 binding antibody is MBG453, or a functional fragment thereof.

71. The method of claim 67, wherein the IL-1β binding antibody, or functional fragment thereof, is canakinumab, or a functional fragment thereof, and the TIM-3 binding antibody is MBG453, or a functional fragment thereof.

72. The method of claim 67, wherein
a) canakinumab, or a functional fragment thereof, is dosed at about 200 mg about every 3 weeks, or about 250 mg about every 4 weeks; and
b) MBG453, or a functional fragment thereof, is dosed at about 600 mg about every 3 weeks, or about 800 mg about every 4 weeks.

73. A method of treating one or more low risk MDS, as defined by IPSS-R, in a subject in need thereof, comprising
a) administering canakinumab, or a functional fragment thereof, at a dose of 200 mg every 3 weeks in combination with MBG453, or a functional fragment thereof, at a dose of 600 mg every 3 weeks; or
b) administering canakinumab, or a functional fragment thereof, at a dose of 250 mg every 4 weeks in combination with MBG453, or a functional fragment thereof, at a dose of 800 mg every 4 weeks.

74. The method of claim 54, wherein the method is a method of preventing the one or more MDS, and wherein the one or more MDS arose from antecedent clonal hematopoiesis of indeterminate potential (CHIP).


SEQUENCE LISTING (1010 sequences)

SEQ ID NO: 1-505 - skipped (505 entries)

SEQ ID NO: 506 - protein, 117 aa, Artificial Sequence
  Synthetic polypeptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507-515 - skipped (9 entries)

SEQ ID NO: 516 - protein, 113 aa, Artificial Sequence
  Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517-519 - skipped (3 entries)

SEQ ID NO: 520 - protein, 113 aa, Artificial Sequence
  Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521-605 - skipped (85 entries)

SEQ ID NO: 606 - protein, 120 aa, Artificial Sequence
  Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607-615 - skipped (9 entries)

SEQ ID NO: 616 - protein, 107 aa, Artificial Sequence
  Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617-619 - skipped (3 entries)

SEQ ID NO: 620 - protein, 120 aa, Artificial Sequence
  Synthetic polypeptide
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621-623 - skipped (3 entries)

SEQ ID NO: 624 - protein, 107 aa, Artificial Sequence
  Synthetic polypeptide
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625-705 - skipped (81 entries)

SEQ ID NO: 706 - protein, 125 aa, Artificial Sequence
  Synthetic polypeptide
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707-717 - skipped (11 entries)

SEQ ID NO: 718 - protein, 107 aa, Artificial Sequence
  Synthetic polypeptide
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719-723 - skipped (5 entries)

SEQ ID NO: 724 - protein, 125 aa, Artificial Sequence
  Synthetic polypeptide
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725-729 - skipped (5 entries)

SEQ ID NO: 730 - protein, 107 aa, Artificial Sequence
  Synthetic polypeptide
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731-761 - skipped (31 entries)

SEQ ID NO: 762 - protein, 447 aa, Artificial Sequence
  Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  Synthetic polypeptide
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  Synthetic polypeptide
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766-800 - skipped (35 entries)

SEQ ID NO: 801 - protein, 5 aa, Artificial Sequence
  Synthetic polypeptide
SYNMH

SEQ ID NO: 802 - protein, 17 aa, Artificial Sequence
  Synthetic polypeptide
DIYPGNGDTSYNQKFKG

SEQ ID NO: 803 - protein, 9 aa, Artificial Sequence
  Synthetic polypeptide
VGGAFPMDY

SEQ ID NO: 804 - protein, 7 aa, Artificial Sequence
  Synthetic polypeptide
GYTFTSY

SEQ ID NO: 805 - protein, 6 aa, Artificial Sequence
  Synthetic polypeptide
YPGNGD

SEQ ID NO: 806 - protein, 118 aa, Artificial Sequence
  Synthetic polypeptide
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  Synthetic polynucleotide
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  Synthetic polypeptide
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  Synthetic polynucleotide
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  Synthetic polypeptide
RASESVEYYGTSLMQ

SEQ ID NO: 811 - protein, 7 aa, Artificial Sequence
  Synthetic polypeptide
AASNVES

SEQ ID NO: 812 - protein, 9 aa, Artificial Sequence
  Synthetic polypeptide
QQSRKDPST

SEQ ID NO: 813 - protein, 11 aa, Artificial Sequence
  Synthetic polypeptide
SESVEYYGTSL

SEQ ID NO: 814 - protein, 3 aa, Artificial Sequence
  Synthetic polypeptide
AAS

SEQ ID NO: 815 - protein, 6 aa, Artificial Sequence
  Synthetic polypeptide
SRKDPS

SEQ ID NO: 816 - protein, 111 aa, Artificial Sequence
  Synthetic polypeptide
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  Synthetic polynucleotide
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  Synthetic polypeptide
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  Synthetic polynucleotide
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  Synthetic polypeptide
DIYPGQGDTSYNQKFKG

SEQ ID NO: 821 - protein, 6 aa, Artificial Sequence
  Synthetic polypeptide
YPGQGD

SEQ ID NO: 822 - protein, 118 aa, Artificial Sequence
  Synthetic polypeptide
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  Synthetic polynucleotide
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  Synthetic polypeptide
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  Synthetic polynucleotide
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  Synthetic polypeptide
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  Synthetic polynucleotide
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  Synthetic polypeptide
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  Synthetic polynucleotide
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  Synthetic polypeptide
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  Synthetic polypeptide
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  Synthetic polypeptide
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  Synthetic polypeptide
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  Synthetic polypeptide
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903-919 - skipped (17 entries)

SEQ ID NO: 920 - protein, 124 aa, Artificial Sequence
  Synthetic polypeptide
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-1000 - skipped (79 entries)

SEQ ID NO: 1001 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1002 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1003 - protein, 114 aa, Artificial Sequence
  Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1004 - protein, 297 aa, Artificial Sequence
  Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 114 aa, Artificial Sequence; identical to SEQ ID NO: 1001
  Synthetic polypeptide

SEQ ID NO: 1006 - protein, 77 aa, Artificial Sequence
  Synthetic polypeptide
ITCPPPMSVEHADIWVKSYSLYSRERYICNSGFKRKAGTSSLTECVLNKATNVAHWTTPSLKCIRDPALVHQRPAPP

SEQ ID NO: 1007 - protein, 214 aa, Artificial Sequence
  Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1008 - protein, 448 aa, Artificial Sequence
  Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1009 - protein, 445 aa, Artificial Sequence
  Synthetic polypeptide
QVQLQESGPGLVKPSQTLSLTCSFSGFSLSTSGMGVGWIRQPSGKGLEWLAHIWWDGDESYNPSLKSRLTISKDTSKNQVSLKITSVTAADTAVYFCARNRYDPPWFVDWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 1010 - protein, 214 aa, Artificial Sequence
  Synthetic polypeptide
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC